(19) United States
(12) Patent Application Publication
(10) Pub. No.: US 2011/0144125 A1
(43) Pub. Date: Jun. 16, 2011

(54) Anti-Arrhythmia Agents, Methods of Their Use, Methods of Their Identification and Kits Therefore

(75) Inventor: Paul Wolkowicz, Hoover, AL (US)

(73) Assignee: THE UAB RESEARCH FOUNDATION, Birmingham, AL (US)

(21) Appl. No.: 13/058,826

(22) PCT Filed: Aug. 14, 2009
(86) PCT No.: PCT/US09/53936

§ 371 Date: Feb. 11, 2011

Related U.S. Application Data
(60) Provisional application No. 61/088,917, filed on Aug. 14, 2008.

(51) Int. Cl. A61K 31/352; A61K 31/11; A61K 31/365; A61K 31/05; A61K 31/495; A61K 31/4164; A61P 9/06

(52) U.S. Cl. 514/255.03; 514/456; 514/700; 514/450; 514/733; 514/396

(57) ABSTRACT

The disclosure describes an animal model for simulating cardiac arrhythmia. Methods of discovering new anti-arrhythmia drugs using the model are described. Novel anti-arrhythmia agents are provided, as are pharmaceutical compositions made from the agents. Methods of inhibiting spontaneous mechanical activity in myocardially-derived biological systems, and methods of treating and preventing cardiac arrhythmia based on novel anti-arrhythmia agents are described. Kits for performing the above methods are also described.

The present invention claims benefit to U.S. Provisional Application No. 61/088,917, filed Aug. 14, 2008.

### FIELD OF THE DISCLOSURE

The present disclosure relates to agents that affect the activity of cardiac muscle. More particularly, the present disclosure relates to agents that prevent and/or inhibit cardiac arrhythmia, methods of identifying such agents, and methods of using such agents. Methods of diagnosing arrhythmia and disease related to arrhythmia as well as kits for the practice of the disclosed methods are also provided.

### BACKGROUND

Atrial and ventricular arrhythmias are cardiac electromechanical activities that occur independently of normal rhythmic heart function. Some types of arrhythmias that are known to occur (in increasing order of severity) are single ectopic beats (pre-mature contractions), rapid prolonged ectopic activity (tachycardia), and “disorganized” rapid ectopic activity (fibrillation). Cardiac arrhythmias are a leading cause of death and disability in industrialized countries, a leading cause of premature death, and a major health care cost. They are a major clinical burden and account for one-fourth to one-third of all premature deaths. The frequency of many arrhythmias increases with age and so they will increase their burden on the medical system as the average age of the U.S. population continues to increase.

Arrhythmia in the upper heart chambers (the atria) predisposes to stroke, exacerbates ventricular failure, is increasingly common with age, and is refractory to most non-invasive therapeutic approaches. Arrhythmia in the lower heart chambers (the ventricles) increases in frequency following myocardial infarction and during heart failure, and causes “sudden death,” a common cause of premature mortality.

The heart contains three types of cells that affect its primary physiological purpose, rhythmic contractions that propel blood through the circulatory system: (1) cells that spontaneously generate recurrent electrical signals, a property known as normal automaticity (for example, sino-atrial p cells), (2) cells that conduct these signals throughout the heart, and (3) cells known as myocytes that convert the electrical signals into a contractile event.

Normal myocyte contraction consists of two general phases (FIG. 1). The first is the excitation phase wherein an external electrical stimulus provokes the opening of myocyte plasma membrane sodium channels. Sodium entry depolarizes the myocyte which allows voltage-dependent calcium entry to occur during the subsequent action potential repolarization. Myocyte contraction then occurs in the second phase because of a process known as calcium-induced calcium release. Here the small amount of calcium that enters myocytes during repolarization provokes the release of large amounts of calcium from the sarcoplasmic reticulum (SR), the main myocyte calcium store. SR calcium release occurs via the ryanodine receptor calcium release channel (RyR). The resultant increase in myocyte cytoplasmic calcium activates the troponin C-linked actomyosin system to affect contraction. The SR calcium ATPase (SERCA) protein then transports cytosolic calcium back into the SR lumen, producing muscle relaxation, restoring the SR calcium store, and readying the muscle for the next wave of external stimulation.

FIG. 2 provides a graphical representation of this process. In FIG. 2 myocytes maintain both (1) a resting potential of −70 to −85 mV (negative inside) across their plasma membrane and (2) a ˜10,000-fold gradient of calcium from the outside (˜2 mM) to the myocyte cytoplasm (˜0.00001 mM). Following (3) myocyte excitation (i.e., plasma membrane depolarization), small amounts of extracellular calcium enter the myocyte which (4) trigger the release of calcium from intra-myocyte calcium stores sequestered in the SR. Calcium exits from the SR through the RyR. (5) This released calcium then activates myocyte actin-myosin complexes, producing muscle contraction. (6) Cytoplasmic calcium is subsequently transported back into the SR lumen via the SR calcium ATPase (SERCA) resulting in relaxation to await another wave of depolarization

Myocytes are excitable but non-automatic. That is, myocytes do not normally generate electrical or mechanical activity spontaneously as ‘automatic’ sinoatrial P cells do. Rather, myocytes require an external electrical stimulus to initiate contraction, which is their fundamental physiological role. A clear example showing heart excitability but non-automaticity is presented in FIG. 3A. Here an isolated, superfused rat left atrial appendage contracts only under the influence of a 1 Hz pacing stimulus (1 Hz). When the stimulus is terminated (Rest), this muscle becomes quiescent.

Arrhythmias are disruptions in this normal pattern of excitation and contraction. Arrhythmias arise in all three groups of heart cells but the most medically important ones are those that occur when myocytes generate action potentials or depolarizations that either require or occur independently of an external depolarizing stimulus. These ectopic action potentials or depolarizations initiate SR calcium release followed by abnormal heart contraction. Arrhythmic events that require an external stimulus are triggered activity while those that do not are termed automatic events like tachycardias. Both arise from disrupted myocyte calcium homeostasis.

Because most arrhythmias result from altered intra-myocyte calcium handling, pharmaceuticals aimed at managing the action potential may not affect the primary cellular cause of the arrhythmic event. Consequently few classes of pharmaceuticals that modulate the myocardial action potential effectively prevent or reverse atrial or ventricular arrhythmic activity. Indeed, clinical studies have shown that these anti-arrhythmic agents themselves can be pro-arrhythmic, increasing patient mortality and limiting their effectiveness as anti-arrhythmic therapies. Thus, few pharmaceuticals effectively prevent or reverse clinically relevant forms of arrhythmias without significant side-effects on the normal electrical activity of the heart. One likely reason for this paucity of effective anti-arrhythmic pharmaceuticals may be that agents which target the myocyte proteins or the myocyte processes that underlie arrhythmias have not yet been identified.

The prior art is lacking in compounds and methods to effectively treat and/or prevent cardiac arrhythmias, despite a long-felt need for such compounds and methods. Despite the seriousness of the disease, and the mortality and morbidity associated therewith, the prior art has failed to develop and implement consistent treatments for therapeutic intervention. In the same vein there is a long standing need for more efficient and rapid methods of screening potential anti-arrhythmia drugs. Current methods are slow and costly and as noted above, may not target the physiologically relevant targets. The present disclosure addresses these long standing problems in the art.

### BRIEF DESCRIPTION OF THE DRAWINGS

FIG. 1. Schema of the two phase of myocyte contraction: Normal myocytes have a stable resting membrane potential of ˜−85 mV (Left black line). Upon depolarization (lightning bolt), the myocyte sodium channels open which effects depolarization to ˜+30 mV (Phase 0). Potassium and calcium channels then open to initiate repolarization (Phases 2 & 3). During this time extracellular calcium enters the myocyte through the slow calcium channel, binds to the ryanodine receptor and provokes calcium release from the myocyte sarcoplasmic reticulum calcium stores (line 2). Calcium binding to the myocyte myofilaments causes muscle contraction (line 3). After a brief time (˜100 ms), cytoplasmic calcium re-enters the sarcoplasmic reticulum via the calcium SR ATPase (SERCA). This lowers cytosolic calcium to produce muscle relaxation. Upon completion of repolarization (Phase 4), the myocyte awaits a new depolarizing impulse.

FIG. 2. Normal myocyte excitation-contraction coupling: Schema shows a myocyte with (1) a resting membrane potential of ˜−80 mV and (2) a 10,000-fold calcium gradient. (3) The arrival of a wave of excitation originating from the sino-atrial node (SAN) allows a small amount of calcium entry via the L-type calcium channel (LTCC) which (4) induces the release of large amounts of calcium from the sarcoplasmic reticulum (SR). (5) Released calcium triggers contraction while (6) subsequent calcium reuptake into the SR by the SERCA produces relaxation.

FIG. 3. Experimental examples of (3A) normal myocardial excitation-contraction coupling, (3B) triggered activity, (3C) automatic activity and (3D) action potentials: Rat left atrial appendages were isolated and superfused in Krebs-Henseleit (KH) buffer at 30° C. (3A) These normal muscles produce mechanical force (upward deflection) when exposed to an external 1 Hz stimulus (1 Hz). These muscles do not contract in the absence of stimulation (Rest). Thus normal left atrial appendage is not automatic. (3B) Appendages exposed to sea anemone toxin Type II (ATXII) exhibit triggered mechanical events. That is, in the face of a 1 Hz pacing stimulus multiple mechanical events (e.g., ‡) are elicited following a single stimulus. These ectopic events arise because of afterdepolarizations and are triggered events because they require a previous depolarization to occur; that is no ectopic activity arises in the absence of pacing (Rest). (3C) Normal heart muscle can also generate automatic activity. In this exemplar of the model, left atrial appendages, treated with 20 μM 2-aminoethoxydiphenyl borate (2-APB) produce repeated, spontaneous contractile events in the absence of a pacing stimulus (Rest; *). This activity is designated spontaneous mechanical activity (SMA). (3D) Action potentials obtained from (left panel) an untreated rat left atrial appendage paced at 1 Hz, (middle panel) an appendage treated with 30 nM ATXII and paced at 1 Hz, and (right panel) an unpaced appendage treated with 20 μM 2-APB. ATXII prolongs action potential duration and provokes early afterdepolarizations while appendages treated with 2-APB produce normal-looking action potentials in the absence of pacing stimulation (automatic activity).

FIG. 4. Examples of early afterdepolarizations and delayed afterdepolarizations: (4A) Early afterdepolarizations (EADs) arise during the repolarization phase of the action potential (Phases 2 and 3). If EADs are of a sufficient magnitude, they will trigger an ectopic mechanical event. (4B) Delayed afterdepolarizations (DADs) arise when the myocyte has returned to its resting potential. Similar to EADs, prior art states that if a DAD can raise the resting potential of a myocyte to a value great enough to open sodium channels then the myocyte will initiate an ectopic action potential that produces an attendant ectopic mechanical event. These two types of afterdepolarizations are responsible for triggered arrhythmic activity.

FIG. 5. Sea anemone toxin Type II (ATXII) binds to the sodium channel and increases the time required for it to close; that is, it increases the late sodium current. As a result, ATXII (i) markedly prolongs the action potential duration and (ii) increases myocyte sodium content which (iii) effects myocyte calcium loading via the sodium-calcium exchanger. As a result of this accepted sequence of events, myocytes treated with ATXII produce EADs which produce triggered mechanical activity.

FIG. 6. Polyphenols including EGCG, gossypol, epigallocatechin (EGC), resveratrol, and quercetin prevent the electromechanical instability that occurs in the disclosed experimental model of abnormal automaticity. (6A) Left atrial automatic mechanical activity when treated with 2-APB alone (upper panel) or with EGCG then with 2-APB (lower panel). (6B) Rate of spontaneous mechanical activity (SMA) of untreated and treated cardiac muscle.

FIG. 7A. The inositol 1,4,5-trisphosphate receptor (IP3R) regulates calcium release or leak from a myocyte sub-sarcolemmal calcium store. The functional activity of this channel is itself regulated by bcl-2. Binding of bcl-2 to the receptor or the activation of a pathway involving bcl-2 or another IP3R active molecule or polypeptide [symbol] provokes calcium leakage from this pool which leads to the expression of automatic arrhythmic activity.

FIG. 7B. Prior art has shown that >10 μM 2-APB activates cell calcium entry via the voltage-independent store-operated calcium channel (SOC). Calcium entry through the SOC channel coupled with calcium leak from an internal myocyte calcium store including but not limited to the IP3R store modifies the functional activity of a cardiac sodium channel so as to induce its opening in the absence of an external stimulus. This change in sodium channel activity results either from a calcium-dependent post-translation modification or from the association of a novel protein with a sodium channel (crescent). These calcium-driven changes alter a cardiac sodium channel from its normal transient form (I_{NaT}; dark ellipsoid) to a form with persistent channel activity (I_{NaP}; light ellipsoid) that is known to activate automatic activity (see Clay J R, “On the persistent sodium current in squid giant axons”, J Neurophysiology 89:64-644 (2003), which is hereby incorporated by reference for such teaching). This automatic sodium channel activity lies downstream of bcl-2's site-of-action in the model presented in FIG. 7A.

FIG. 8. Bcl-2 regulates myocyte apoptosis via a pathway that has been described in terms of non-excitable and excitable cells.

FIG. 9. Decreasing superfusate chloride prevents SMA: Upper panel: Mechanical function typical of 0.1 Hz paced left atrial appendage washed with 250 ml of KH containing 30 mM total Cl and a constant Na of 145 mM. Fifteen minutes after washing, this atrium was exposed to 15 μM 2-APB (2APB) for 15 min. Lower panel: The number of muscle preparations exhibiting SMA at each concentration of superfusate Cl (▪, n=6-8 total preparations) relative to KH (126 mM Cl) where all preparations exhibited SMA. No error bars are reported as these experiments tested whether preparations did or did not exhibit SMA.

FIG. 10. Decreasing superfusate sodium prevents SMA: Upper panel: Mechanical function typical of 0.1 Hz paced left atrial appendage washed with 250 ml of KH containing 109 mM Na and constant Cl of 126 mM. Fifteen minutes after washing, this muscle was exposed to 15 μM 2-APB (2APB) for 15 min. Lower panel: The number of muscle preparations exhibiting SMA (e.g. FIG. 3C, Rest) at each concentration of superfusate Na (▪, n=6-8 total preparations) relative to KH (145 mM Na) where all preparations exhibited SMA. No error bars are reported as these experiments tested whether preparations did or did not exhibit SMA.

FIG. 11. Increasing superfusate potassium reverses SMA: Upper panel: Mechanical function typical of 0.1 Hz paced atrial appendage superfused in KH and exposed to 15 μM 2-APB (2APB). Following the appearance of SMA, increasing amounts of KCl were added to the superfusate (Solid line: KCl). Lower panel: (▪) The number of preparations (n=7 total preparations) exhibiting prolonged SMA relative to KH (5.8 mM KCl) where all preparations exhibited SMA; (∘) Basal force of contraction measured in untreated atria (n=7 total) exposed to increasing KCl relative to the basal force measured in normal KH (5.8 mM KCl) (i.e, Initial force).

FIG. 12. 2-APB induces spontaneous mechanical events (SMEs) in isolated, superfused rat left atrial appendage: (12A) Left panels: Mechanical function typical of isolated, superfused rat left atrial appendage paced at 3 Hz; Right panels: Mechanical function typical of 3 Hz paced appendage exposed to 15 μM 2-APB for 15 min. (12B) The number of SMEs, defined as spontaneous increases in force (Upper right panel: *), that occurred per min in muscles superfused in KH, paced at 3 Hz, and exposed to 0 (n=5), 2 (n=5) 7.5 (n=8), 15 (n=10), or 22 μM (n=8) 2-APB. The structurally/functionally related compound diphenyl boronic anhydride gave similar results (Data not shown). Exposure time was constant at 15 min. Mean±SEM are reported. *=p<0.05 vs. 0 μM 2-APB; ‡=p<0.05 vs. 15 μM 2-APB.

FIG. 13. 2-APB induces SMA when diastolic interval is prolonged: (13A) Mechanical function typical of 0.1 Hz paced atrial appendage (n=6). (13B) Mechanical function typical of 0.1 Hz paced muscle (n=8) exposed to 7.5 μM 2-APB for 15 min. One or more SMEs (*) occurred in each muscle. (13C) Mechanical function typical of 0.1 Hz paced appendage (n=8) exposed to 22 μM 2-APB for 15 min. Isolated SMEs gave way to prolonged SMA (right part of panel).

FIG. 14. Decreasing superfusate sodium or DIDS reverses SMA: (14A) Mechanical function typical of 0.1 Hz paced left atrial appendage superfused in KH (145 mM Na) and exposed to 22 μM 2-APB (2-APB). Following the appearance of SMA (darkened areas that result from multiple automatic contractile events) this appendage was washed with 250 ml of KH containing 82 mM Na and 22 μM 2-APB. Approximately 23 min later a 1 min period of rest (Rest) was imposed on this preparation followed by a resumption of stimulation in order to obtain its maximum force of contraction, its post-rest potentiation response (*; PRP). (14B) Mechanical performance typical of 0.1 Hz paced appendage superfused in KH and exposed to 22 μM 2-APB. Following SMA, 400 μM DIDS was added to the superfusate to block anion transporters that may depolarize left atrial appendage myocytes. Approximately 23 min later a 1 min period of rest (Rest) was imposed and appendage PRP (*) was obtained.

FIG. 15. DIDS reverses SMA: Upper panel: Mechanical function typical of 0.1 Hz paced atrial appendage superfused in KH and exposed to 15 μM 2-APB (2APB). Following the appearance of SMA, left atria were titrated with 100 to 300 μM DIDS (Solid lines: DIDS). Lower panel: The number of muscle preparations (n=7 total preparations) exhibiting prolonged SMA after a 3-5 min incubation at each concentration of DIDS relative to KH where all preparations exhibited SMA.

FIG. 16. 2-APB significantly decreases the maximum force of atrial contraction under conditions that suppress SMA: −2APB KH: Maximum forces of atrial contraction (PRP) (n=6) were obtained from a 1 min PRP performed at the end of a 35 min superfusion. −2APB ΔNa DIDS: Maximum forces of atrial contraction were measured 23 min after lowering superfusate sodium to 82 mM (ΔNa; n=8) or adding 400 μM DIDS (DIDS; n=8). +2APB ΔNa:DIDS: Maximum forces of atrial contraction were measured in preparations exposed to 22 μM 2-APB 23 min after lowering superfusate sodium (ΔNa; n=8) or adding DIDS (DIDS; n=8). These values were compared to PRPs performed ˜10 min prior to the addition of 2-APB. Mean±SEM. *=p<0.05 versus (−)₂-APB. ‡=p<0.05 versus ΔNa.

FIG. 17. 2-APB induces left atrial appendage SMEs in the absence of electrical stimulation: (17A) Mechanical function typical of 3 Hz paced atria (n=5) (3 Hz stimulus) subjected to 5 min of rest (Rest). Potentiation of mechanical force (*) occurred with re-initiation of pacing. (17B) Mechanical function typical of 3 Hz paced atria (n=6) (3 Hz stimulus) exposed to 15 μM 2-APB for 10 min (←2APB) followed by a 5 min rest. SMEs (§) occurred in all 2-APB-treated atria. Post-rest potentiation (*) was blunted in all 2-APB-treated

FIG. 18. Mechanical parameters of normal, untreated left atrial appendage and appendages undergoing SMA: Left atrial appendages were paced at 3 Hz and control values were obtained; 2-APB values were obtained during SMA. Measures=number of independent measurements in each group; TPT=time to peak tension; T0.9R=time to 90% relaxation; T0.5R=time to 50% relaxation. Data are mean±SEM.

FIG. 19. Induction of SMA and tachycardic automatic activity in isolated left atrial appendage. (19A) Typical mechanical function of an isolated rat left atrial appendage paced at 0.1 Hz and superfused at 30° C. in KH buffer. In the absence of pacing (Rest; inset) this muscle is quiescent. (19B) A second left atrial appendage superfused and paced as in (A). This muscle was exposed to 22 μM 2-APB where indicated. After a few minutes this muscle persistently produces mechanical events that occur independently of external stimulation (Rest; inset). (19C) Muscle identical to (19B) except that it was treated with 30 nM isoproterenol (Iso). Under this condition isolated appendages produce spontaneous activity in the absence of pacing (Rest; inset) at a rate of ˜230 contractions/min. This spontaneous tachycardic activity (STA) exceeds the rate of normal right atrial pacemaker-driven function, ˜170 contractions/min at 30° C.

FIG. 20. Ranolazine blocks ectopic activity: (20A) Titration curve of ranolazine reversal of SMA in superfused left atrial appendage. Superfused appendages (n=7) were treated with 2-APB and following the appearance of SMA they were titrated with increasing concentrations of ranolazine for 3-5 min at any concentration. Rates of SMA in the absence of pacing were recorded. (20B) Typical raw mechanical data for an appendage treated with 2-APB and then with 0, 10 or 80 μM ranolazine. All data are in the absence of pacing. (20C) Rate of STA in superfused appendages treated with 2-APB and 300 nM (−) BayK 8644 before (□) and after (▪) a 10 min exposure to 80 μM ranolazine.

FIG. 21. Flecainide suppresses SMA: (21A) Upper panel: Left atrial appendage paced at 0.1 Hz. Middle panel: Same appendage superfused with 22 μM 2-APB; SMA occurs in this muscle. Lower panel: Appendage treated with 65 μM flecainide following the appearance of SMA. (21B) Summary of data for left atrial appendages (n=8) that were exposed to 22 μM 2-APB and, following the appearance of SMA, were titrated with increasing concentrations of flecainide. The % of atrial preparations exhibiting SMA (SCA) was recorded after a 5 min exposure to any concentration of flecainide.

FIG. 22. Pharmacological activation of STA: Condition=Experimental condition; Iso=30 nM isoproterenol; Forskolin=3 μM forskolin; 2APB=20 μM 2APB; Bay K=300 nM (−)Bay K 8644; FPL=300 nM FPL-64176; Ryndn=600 nM ryanodine; Ouab=120 μM ouabain; Pace=pacing rate; n=number of independent measures; Force=(mg force per g atrial wet weight (gww)), prior to (Pre) or following (Post) a 10 min exposure to the agent; SMA Rate=SMEs per minute; TPT=time to peak tension; T0.5R=time to 50% relaxation; T0.9R=time to 90% relaxation. Data are mean±SEM. *=p<0.05 vs. Pre-treatment force; ‡=p<0.05 vs. Untreated; §=p<0.05 vs. corresponding control.

FIG. 23. Lowering superfusate sodium reverses STA: (23A) The mechanical function of an unpaced left atrial appendage (n=9) undergoing STA in the presence of 300 nM Bay K 8644 and 20 μM 2-APB. Superfusate sodium was reduced from 145 to 82 mM where indicated. (23B) The mechanical function of the unpaced left atrial appendage in (23A) exposed to BayK 8644, 2-APB, and 82 mM sodium. 0.1 Hz pacing was reinstituted where indicated (0.1 Hz).

FIG. 24. Rat left atria contain HCN2 and HCN4 cDNAs: Total RNA was extracted from 3 rat right and 3 rat left atria. RT-PCR analyses for HCN1-4 were performed as described in Methods. Bar graphs summarize these amplifications relative to a cyclophilin control. Right atria=RA; □. Left atria=LA; [symbol] Data are mean±S.E.M.

FIG. 25. Zatebradine decreases the frequency of STA: (25A) The mechanical function of a 3 Hz-paced rat left atrial appendage (n=9) subjected to ˜15 sec of rest. (25B) The mechanical function of a 3 Hz-paced appendage (n=9) exposed to 20 μM 2-APB (2-APB) for 10 min prior to rest. (25C) The mechanical function of a 3 Hz-paced appendage (n=9) treated with 300 nM (−)BayK 8644 for 5 min and 2-APB for 10 min. Discordant mechanical events occur with 3 Hz pacing (‡). (25D) The mechanical function of a 3 Hz-paced appendage (n=9) treated with BayK 8644 and 2-APB for 10 min and then with 70 μM zatebradine (ZTB) for 10 min.

FIG. 26. (26A) Concentration dependence of zatebradine suppression of STA: Nine 3 Hz-paced left atrial appendages (∘) were exposed to 2-APB and BayK 8644. After the appearance of STA, appendages were titrated with 0 to 100 μM zatebradine and its effect on the frequency of STA was recorded 3-5 min after any addition. Nine rat right atria (▪) were titrated with 0 to 100 μM zatebradine. The effect of zatebradine on right atrial contraction frequency was recorded. % of the initial rate of STA (left atria) or normal automatic contraction (right atria) are reported. (26B) ZD-7288 suppresses STA: Seven 3 Hz-paced left atrial appendages (∘) were exposed to 2-APB and BayK 8644. After the appearance of STA, muscles were titrated with 0 to 100 μM ZD-7288 and its effect on STA was recorded 3-5 min after any addition. Seven rat right atria (▪) were titrated with 0 to 100 μM ZD-7288. The effect of ZD-7288 on right atrial contraction frequency was recorded. % of the initial rate of STA (left atria) or normal automatic contraction (right atria) are reported. All data are mean±S.E.M.

FIG. 27. Induction of SMA, STA, and chaotic ectopy in rat right ventricular muscle strips: (27A) Typical mechanical function of right ventricular muscle strip superfused at 30° C. and paced at 0.5 Hz. (27B) Similar superfused right ventricular muscle strip exposed for ˜10 min to 22 μM 2-APB. SMA occurs in this muscle (‡). (27C) Right ventricular muscle strip treated with 30 nM isoproterenol and 2-APB show STA in the absence of a 1 Hz pacing stimulus (Rest). Ventricular muscle treated in this way shows disorganized mechanical activity in the presence of 1 Hz pacing (e.g., §).

FIG. 28. Temperature-dependence of left atrial appendage STA and right atrial normal automaticity. (28A) Rat left atrial appendage (▪; n=6-8) superfused at 30° C., paced at 0.1 Hz, treated for 5 min with 300 nM (−)BayK 8644 and then for 10 min with 22 μM 2-APB. Pacing was stopped and rates of STA (Contraction rate) were recorded. A second group of appendages (n=8) were superfused at 23° C. and treated identically to the first. 10 min later STA was measured without pacing and muscle bath temperature was increased to 37° C. Maximum STA rates were measured over 10 min (Contraction rate). Rat right atria (▴; n=8) were superfused at 30° C. and rates of pacemaker-driven mechanical contraction were recorded. A second group was superfused at 23° C. and their contraction rates were recorded; muscle bath temperature was increased to 37° C. and maximum contraction rates were measured over 10 min. (28B) Upper: Mechanical function of a superfused rat right atrium measured without pacing at 37° C. Middle: Mechanical function of a 0.1 Hz-paced, superfused left atrial appendage treated for 5 min with 300 nM (−)BayK 8644 alone at 37° C. Function is measured here without pacing. Lower: Mechanical function of a rat left atrial appendage treated as per the second group in (28A) and measured without pacing at 37° C.

FIG. 29. Induction of chaotic ectopy in isolated left atrial appendage and right ventricular muscle strips: (29A) Typical mechanical function of a rat left atrial appendage superfused at 37° C., paced at 5 Hz, and exposed to 22 μM 2-APB and 300 nM BayK 8644 (BayK). All muscles exhibit chaotic, disorganized mechanical activity (§) when paced at this physiological rate. In the absence of pacing (Rest) muscles show only STA. (29B) Typical mechanical function of a rat right ventricular muscle strip superfused at 30° C., paced at 3 Hz, and exposed to 22 μM 2-APB and 30 nM isoproterenol (Isoprel). All such muscles exhibit chaotic mechanical function (§) in the presence of pacing. In the absence of pacing (Rest) these muscle show only STA.

FIG. 30. Bcl-2 inhibitors prevent left atrial appendage SMA: Rat left atrial appendages were superfused and left untreated (▪) or were pre-treated with 80 μM 2-methoxy antimycin A (Δ) or 30 μM HA14-1 (). Muscles then were titrated with increasing concentrations of 2-APB; SMA was measured after a 10 min exposure to any concentration of 2-APB.

FIG. 31. (31A) EGCG reversal of SMA: The mechanical function of a rat left atrial appendage superfused at 30° C. and paced at 0.1 Hz (0.1 Hz). This muscle was exposed to 20 μM 2-APB (2-APB) and following the appearance of SMA the pacing stimulus was stopped (Rest). Approximately 1 min later 40 μM EGCG was added to the muscle bath and SMA ceased. Approximately 3 min later the 0.1 Hz pacing stimulus was reinstituted and normal LAA excitation-contraction coupling resumed. (31B) Summary of polyphenol reversal of SMA: Rat left atrial appendages were exposed to 20 μM 2-APB for 10 min and rates of SMA were recorded (100% Initial). Appendages then were titrated with up to 5 concentrations of the bcl-2 antagonists EGCG (∘), gossypol (Gssy; ▪), and HA14-1 (▴). Rates of ectopic activity were recorded ˜10 min after the addition of any concentration of bcl-2 antagonist.

FIG. 32. Chemical structure of ABT-737.

FIG. 33. SKF-96365 reverses and prevents SMA and STA: We tested whether the store-operated calcium channel (SOC), a bcl-2 target, participates in SMA or STA. Upper panel, left side: A 0.1 Hz-paced left atrial appendage was treated with BayK 8644 and 2-APB to induce STA and then ˜50 μM SKF-96365 was added to the muscle bath (SKF-96365). Upper panel, middle: SKF-96365 reversed STA automaticity, Upper panel, right side: Restored normal pacing-induced mechanical contractions occurred in a one-to-one manner. Lower panel: Summary of SKF-96365 reversal of STA. Other experiments (data not shown) reveal that this SOC blocker prevents STA and that it also prevents and reverses SMA.

FIG. 34. STA occurs under conditions of prolonged action potential duration: Upper panel: Exposing superfused rat left atrial appendages to ˜50 nM ATXII prolongs their action potential duration and induces early afterdepolarizations; Middle panel: These afterdepolarizations precede and are required for aftercontractions. Lower panel: Adding ˜20 μM 2-APB to ATXII-treated left atria induces STA which occurs at a rate comparable to those seen under other conditions of calcium loading that provoke STA. Thus 2-APB provokes automaticity in myocardium with prolonged action potential duration that increases calcium loading.

FIG. 35. STA dos not require ryanodine-sensitive calcium stores: Upper panel: Superfused left atrial appendage treated with BayK 8644 and 2-APB produce STA with normal levels of mechanical function. Lower panel: Treating these appendages with 600 nM ryanodine decreases their mechanical function significantly (compare scales of force) but does not terminate STA. This result indicates that depletion of the ryanodine receptor-linked calcium store does not suppress STA and implies that calcium leaked from this store is not critical to the model of automatic activity claimed in this disclosure.

FIG. 36. STA requires caffeine sensitive calcium stores: Upper panel: Superfused rat left atrial appendage were exposed to ˜25 μM 2-APB to produce SMA and the pacing stimulus was stopped. Middle panel: These unpaced left atria undergoing SMA then were washed with 30° C. aerated KH containing 2-APB and 10 mM caffeine. Caffeine depletes ryanodine-sensitive and -insensitive intracellular calcium stores. Immediately after adding caffeine and in the continued presence of 2-APB, the sporadic rate of SMA increases to the rapid rate of STA. All automatic mechanical activity ceases after 1-3 minutes. Lower panel: These (2-APB & caffeine)-treated left atria respond normally to external pacing stimuli.

FIG. 37. Rapid external pacing can capture STA automaticity: Upper panel: Superfused, paced rat left appendage were treated with 300 nM BayK 8644 and the pacing stimulus was stopped (Rest). Soon thereafter the pacing stimulus was temporarily reinstituted at a rate of ˜5 Hz and this normal, non-automatic muscles was captured in a one-to-one manner. Lower panel: Superfused rat left atrial appendage was exposed to BayK 8644 and ˜25 μM 2-APB to instigate STA. This muscle then was subjected to a burst of ˜5 Hz pacing. Pacing at a rate much faster than the rate of STA captured this muscle in a one-to-one manner. Thus STA is sensitive to external overdrive pacing.

FIG. 38. The STA system interacts with the non-automatic, pacing-dependent system to provoke fibrillation-like activity: (A) Rat left atrial appendages undergoing STA were exposed to a pacing stimulus set to ˜80% of the rate of STA (in this example, ˜2.9 Hz), a 5 ms pulse duration, and 130% of the capture voltage; disorganized, chaotic mechanical activity reminiscent of fibrillation occurs. Increasing the voltage of the 2.9 Hz pacing stimulus to 650% of capture voltage results in mechanical function showing one-to-one capture (right of panel). (B) If the pacing stimulus is turned off, STA again commences a few seconds afterwards. (C) If the muscle is returned from 650% capture voltage to 130% capture voltage, it again descends into fibrillatory like activity. (D) Fibrillation also results when left atrial appendages undergoing STA are subjected to a single high-voltage pulse of >50 ms duration and ˜5-times the capture voltage. (E) Subjecting these fibrillating left atria to a single pulse for >50 ms duration but of ˜10 times the capture voltage restores STA.

### SUMMARY

The present disclosure provides model systems for simulating cardiac arrhythmia, in one embodiment, abnormal automaticity in isolated heart muscle. The present disclosure further provides methods and kits employing the model systems directed to identifying anti-arrhythmic compounds. The present disclosure further provides novel anti-arrhythmic agents, kits comprising the novel anti-arrhythmic agents, and methods of using the novel anti-arrhythmic agents. Additionally, the present disclosure provides methods of treating and preventing arrhythmia. The present disclosure further provides methods of inhibiting spontaneous mechanical activity (SMA) in a myocyte. Still further, the present disclosure provides methods of inhibiting arrhythmia in a cardiac muscle.

It is an objective of the present disclosure to provide a model for arrhythmia and/or SMA in a controlled setting providing easily reproducible results. A novel model system has been developed in which arrhythmia and/or SMA is easily and quickly induced in cardiac muscle, allowing for the rapid screening of anti-arrhythmic agents.

It is a further objective of the present disclosure to provide a model for arrhythmia that simulates the more serious sustained forms of cardiac arrhythmia, such as, but not limited to, tachycardia and fibrillation. It has unexpectedly been discovered that exposure of cardiac muscle to certain agents results in the appearance of these more serious, sustained forms of cardiac arrhythmia. Consequently, this model allows for the rapid screening of potential agents to inhibit tachycardia, fibrillation, and other sustained forms of arrhythmia.

It is a further objective of the present disclosure to provide a model for arrhythmia that simulates either triggered ectopy or automatic ectopy, but not both simultaneously. It has unexpectedly been discovered that exposing cardiac muscle to certain agents under certain conditions causes triggered or automatic ectopic events. Consequently, the model allows for the rapid screening of potential agents that specifically inhibit triggered or automatic ectopy.

It is a further objective of the present disclosure to provide novel anti-arrhythmic agents that are potent inhibitors of arrhythmia and inhibit SMA. It is another objective of the instant disclosure to provide medicaments and pharmaceuticals comprising the novel anti-arrhythmic agents. It is a further objective of the instant disclosure to provide kits comprising the novel anti-arrhythmic agents in any form, including in the form of a medicament or pharmaceutical.

It is a further objective of the present disclosure to provide inhibitors of bcl-2 and bcl-2 targets that may be used as novel anti-arrhythmic agents and to inhibit SMA. The present disclosure has unexpectedly demonstrated that certain inhibitors of bcl-2 or bcl-2 targets are potent inhibitors of arrhythmia and SMA. It is another objective of the instant disclosure to provide medicaments and pharmaceuticals comprising the novel inhibitors of bcl-2 or its targets. It is a further objective of the instant disclosure to provide kits comprising the novel inhibitors of bcl-2 or its targets in any form, including in the form of a medicament or pharmaceutical.

It is a further objective of the present disclosure to provide methods of inhibiting and/or preventing SMA in a myocyte, said method comprising exposing the myocyte to the novel anti-arrhythmic agents disclosed. It is a further objective of the present disclosure to provide methods of treating and/or preventing arrhythmia and to inhibit SMA in a subject, said method comprising administering to a subject a novel anti-arrhythmic agent disclosed herein.

It is a further objective of the present disclosure to provide methods of inhibiting bcl-2 or its targets so as to inhibit and/or prevent SMA in a myocyte, said method comprising exposing the myocyte to the novel anti-arrhythmic agents disclosed. It is a further objective of the present disclosure to provide methods of inhibiting bcl-2 or its targets so as to treat and/or prevent arrhythmia and to inhibit SMA in a subject, said method comprising administering to a subject a novel anti-arrhythmic agent disclosed herein.

It is a further objective of the present disclosure to provide methods and kits for the diagnosis of arrhythmia, risk of arrhythmia, or for a disease state or condition associated with or characterized by increased bcl-2 activity or bcl-2 target activity or by alterations in calcium homeostasis.

### DETAILED DESCRIPTION

Arrhythmias arise or are sustained through two mechanisms; (i) triggered activity and (ii) reentrant activity. The latter involves the abnormal propagation of electrical activity through the heart, and although reentrant activity is critical to sustaining arrhythmias, a triggering event generally precedes reentrant activity. Thus prior art claims that triggered activity is a major, perhaps the predominant, source of abnormal cardiac electro-mechanical activity.

Prior art suggests that triggered activity takes two forms; afterdepolarizations and abnormal automaticity.

Triggered activity results from the abnormal generation of electrical activity in regions of the heart other than the sinoatrial node (SAN) of the right atrium. P cells in the SAN produce the spontaneous electrical activity that drives normal rhythmic atrial and ventricular contractions. Abnormal triggered, so-called ectopic electrical activity is thought to arise from altered calcium homeostasis within heart muscle cells themselves.

Triggered activity involves the production of an abnormal action potential or depolarization in quiescent myocytes. In particular, a triggered arrhythmia occurs when a critical mass of myocytes spontaneously depolarize to produce a single wave or repeated waves of ectopic electrical activity that propagate through the heart.

These ectopic triggered depolarizations arise from specific events within the myocyte. Briefly, it has been believed that triggered activity occurs as a result of aberrant calcium leakage, such as from sarcoplasmic reticulum (SR) stores during the interval between normal, rhythmic myocyte excitation. Leaked SR calcium activates calcium-dependent electrogenic ion transporters in the myocyte plasma membrane. Efflux of leaked calcium via these electrogenic carriers is hypothesized to drive the resting myocyte membrane potential to more positive values until it reaches ˜−65 mV (↓Eₘ (resting membrane potential)). At this range of voltage, quiescent myocytes will generate arrhythmic afterdepolarizations and electrical activity (↓ADs (afterdepolarizations)).

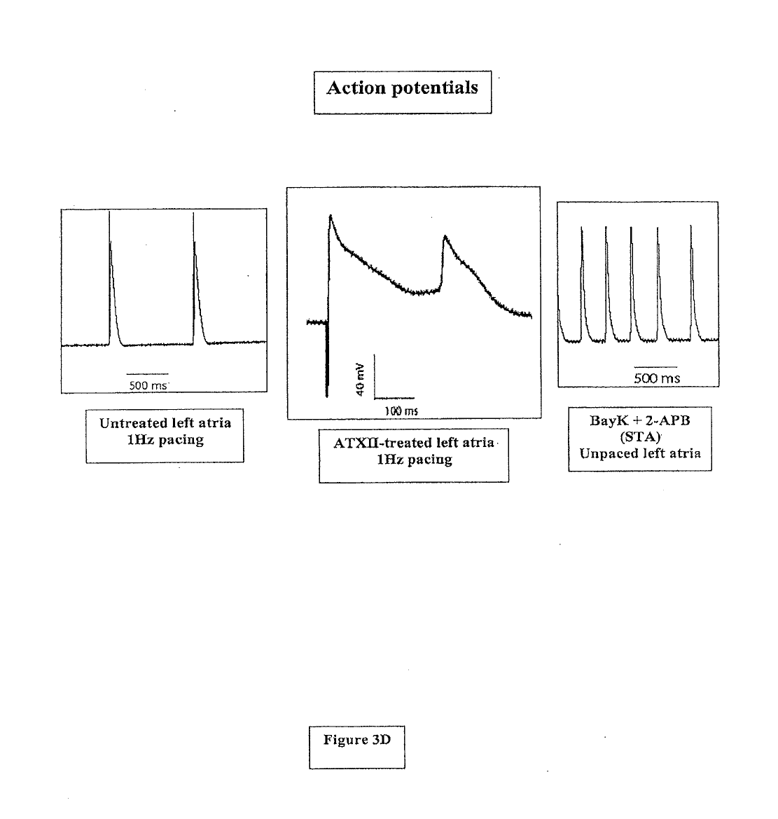

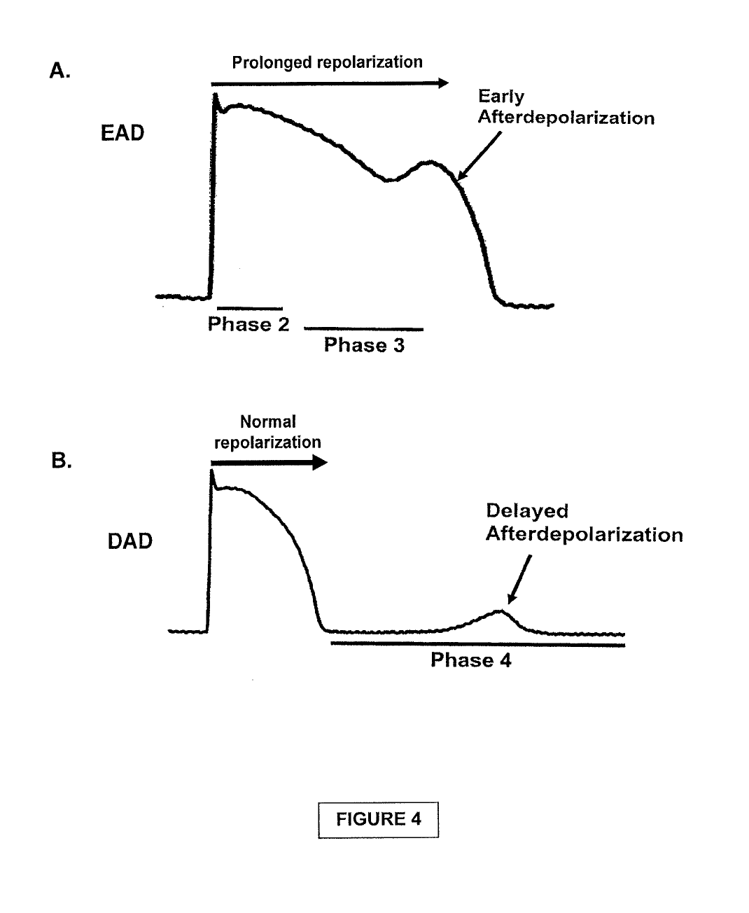

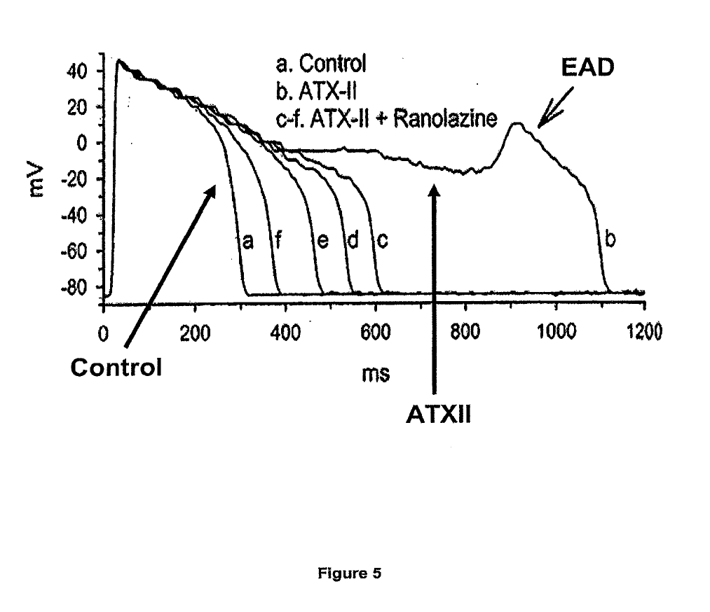

There are two types of afterdepolarizations; early afterdepolarizations (EADs) that occur early during repolarization and delayed afterdepolarizations (DADs) that occur in fully repolarized, resting myocytes (FIG. 4). Both EADs and DADs are defined as “triggered” activities as they require a preceding normal action potential to occur (FIG. 3B). One model to elicit EADs is to expose heart muscle or myocytes to Anemonia sulcata Toxin II (ATX II). This 47 amino acid peptide specifically enhances late sodium current and prolongs the action potential duration (FIG. 3D; middle panel). This leads to calcium loading of heart muscle followed by EADs during phase 2 or phase 3 of the action potential (FIGS. 3D & 5). These EADs, if of sufficient magnitude, produce ectopic mechanical events (FIG. 3B; ‡). ATX II induces triggered activity as stopping the 1 Hz pacing stimulus in left atrial appendages stops both normal and ectopic mechanical activity (FIG. 3B; ‡, Rest).

Prior art suggests that abnormal automaticity is another type of triggered arrhythmia characterized by rapid, repeated spontaneous depolarizations and contractions of non-automatic heart muscle. These events occur at a rate faster than the normal automaticity that is driven by the automatic P cells of the SAN. Prior art claims that afterdepolarizations lead to automatic activity but that once initiated abnormal automaticity does not require external electrical stimulation.

It has been proposed that aberrant arrhythmic myocyte calcium release or leak occurs solely via the ryanodine receptor calcium release channel. The normal ryanodine receptor is impermeable to calcium under resting conditions, maintaining SR calcium stores. It has also been proposed that post-translational hyper-phosphorylation of the SR ryanodine receptor calcium release channel in arrhythmic hearts, increases its open probability during the interval between normal SAN-driven depolarizations. This is proposed to increase arrhythmogenic SR calcium leakage.

Prior art suggest that another mechanism for abnormal automaticity is the increase in or the activation of a persistent sodium current (I_{NaP}) (see Clay J R, “On the persistent sodium current in squid giant axons”, J Neurophysiology 89:64-644 (2003), which is hereby incorporated by reference for such teaching). In this proposed mechanism, persistent sodium channel activity destabilizes the resting electrical state of squid axons (or other cells). This instability leads to automatic firings of a modified sodium channel or a sub-set of sodium channels (see Clay J R and Shrier A, “Action potentials occur spontaneously in squid giant axons with moderately alkaline intracellular pH”. Biological Bulletin 201: 186-194 (2001), which is hereby incorporated by reference for such teaching).

Without being limited to other mechanisms and without limiting the scope of the present disclosure, the present disclosure shows that two other mechanisms alter cell calcium homeostasis which act as sources of arrhythmogenic calcium. First, cells contain a class of calcium release channels known as the inositol 1,4,5-trisphosphate receptor (IP3R). The IP3R can leak calcium from internal stores into the cytoplasm. Post-translational modifications and small protein regulators alter IP3R open probability and the resultant calcium leak. Second, calcium can enter cells via a voltage-independent system known as the store-operated channel (SOC). Prior art in non-excitable cells shows that this channel allows calcium entry in response to the depletion or disruption of intracellular calcium stores.

In non-excitable cells and in excitable cells, such as myocytes, the protein bcl-2 is known to bind to and inactivate a family of proteins that control the intrinsic pathway for programmed cell death (apoptosis). The stability and location of these anti- and pro-apoptotic protein complexes is tightly regulated so as to control cell entry into apoptosis. In non-excitable cells, bcl-2 binding to the endoplasmic reticulum IP3R also regulates the leakage of calcium from endoplasmic reticulum stores. Endoplasmic reticulum calcium store content is an important permissive factor in the intrinsic pathway of programmed non-excitable cell death. Likewise bcl-2 is a known regulator of SOC expression and activity. Without wishing to be bound by any hypothetical model, the present disclosure suggests IP3R calcium leak and voltage-independent calcium entry as important regulators of arrhythmia.

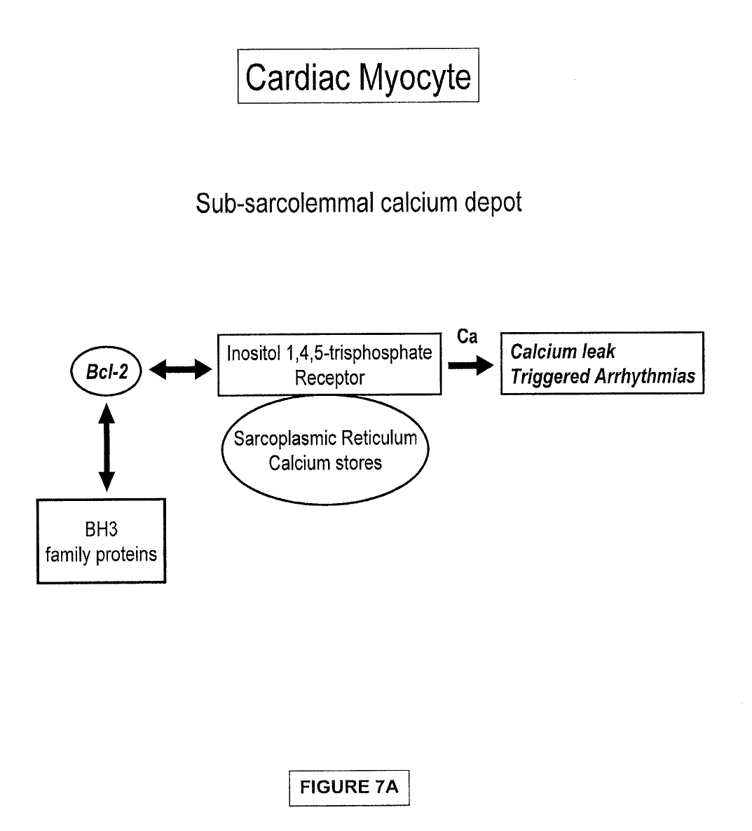

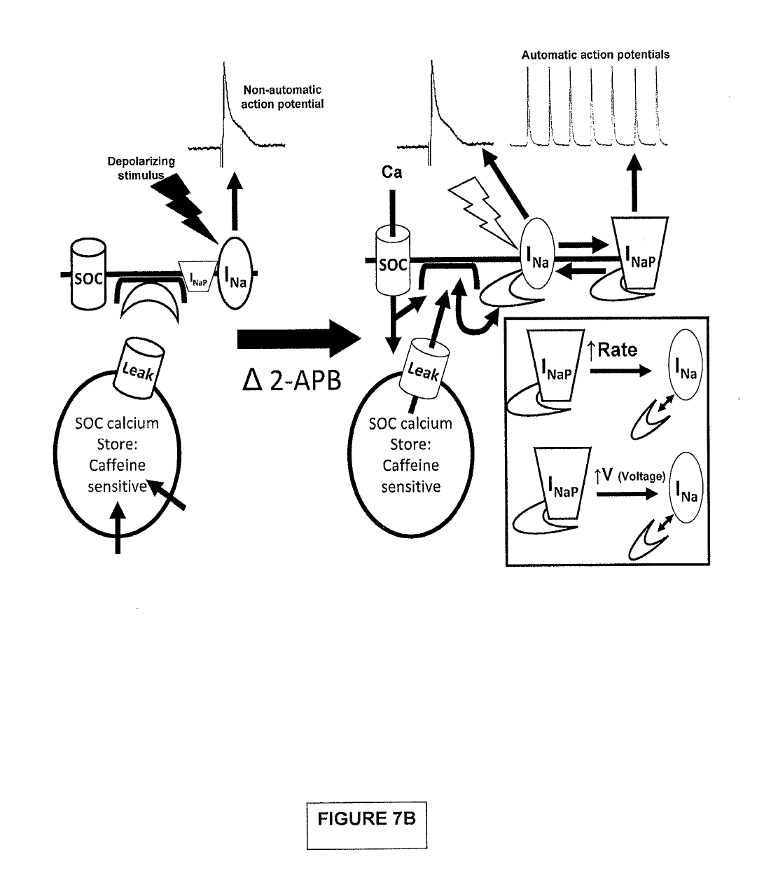

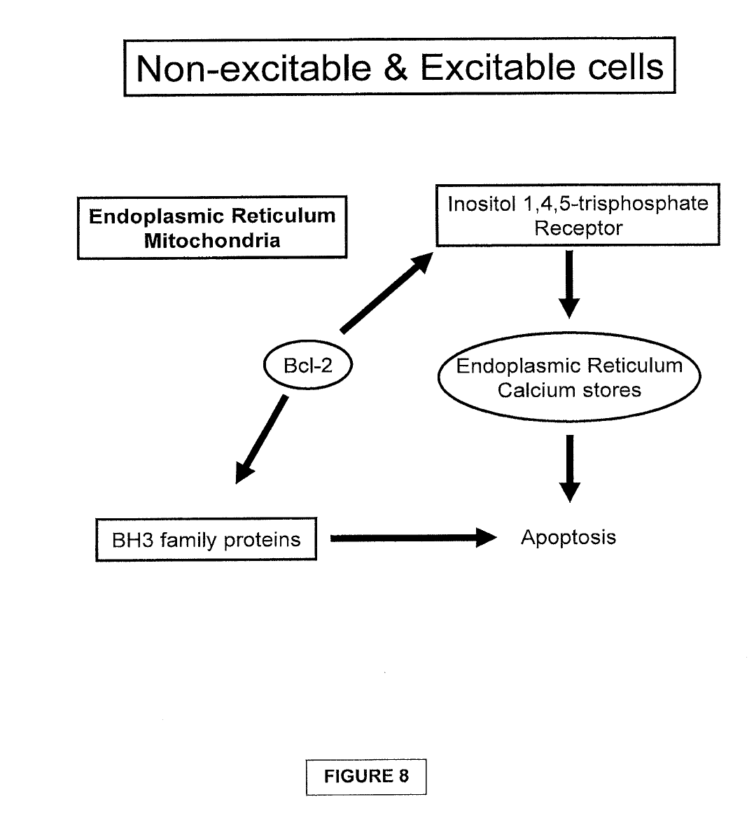

Without being limited to other mechanisms and without limiting the scope of the present disclosure, the present disclosure shows that bcl-2 and bcl-2 targets can stimulate cardiac arrhythmia and/or SM. In particular cardiac bcl-2 controls a pathway to produce cardiac arrhythmias (FIGS. 7A&B and 8). It is hypothesized that cardiac myocytes contain a calcium store (sub-sarcolemmal calcium depot) controlled by the interaction between bcl-2, the IP3R, the SOC and other bcl-2 targets and that calcium entry and leakage from this store elicits automatic activity (FIGS. 7A&B). Cardiac bcl-2 also contributes to myocyte apoptosis through its binding to bcl-2 homology-3 (BH3) family member proteins much like its function in non-excitable cells. It has been shown that other cell factors including but not limited to cytochrome C, calpains, and cell redox affect IP3R calcium leak. Thus regulation of bcl-2 binding to IP3R and the SOC and induction of calcium leak or entry and arrhythmia is proposed to occur because of the interplay among these and other factors. These factors include SOC calcium entry and calcium-linked modification of the properties of a cardiac form of the sodium channel specifically an increase in a cardiac persistent sodium current that leads to automaticity (FIG. 7B).

Bcl-2 is a protein known in the art. The polypeptide exists in at least two splice form variants, resulting from alternative splicing and differ in the c-terminal ends. The human nucleic acid sequence is described in Cleary et al., Cell 47 (1), 19-28 (1986) (Gen Bank ID M147451), which is hereby incorproated by reference for such teaching. Representative amino acid sequences from humans (Homo sapiens; Gen Bank ID. NP_{—}000624.2), chimps (Pan troglodytes; Gen Bank ID. XP_{—}001145537.1), dogs (Canis lupus familiaris; Gen Bank ID. NP_{—}001002949.1), mice (Mus musculus; Gen Bank ID. NP_{—}033871.2), rats (Rattus norvegicus; Gen Bank ID. NP_{—}058689.1), chickens (Gallus gallus; Gen Bank ID. NP 990670.1), and cattle (Bos Taurus; Gen Bank ID. XP_{—}586976.3) are also known.

### A. DEFINITIONS

The terms “prevention”, “prevent”, “preventing”, “suppression”, “suppress” and “suppressing” as used herein refer to a course of action (such as administering a compound or pharmaceutical composition of the present disclosure) initiated prior to the onset of a clinical manifestation of a disease state or condition so as to prevent or reduce such clinical manifestation of the disease state or condition. Such preventing and suppressing need not be absolute to be useful.

The terms “treatment”, “treat” and “treating” as used herein refers to a course of action (such as administering a compound or pharmaceutical composition) initiated after the onset of a clinical manifestation of a disease state or condition so as to eliminate or reduce such clinical manifestation of the disease state or condition. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a condition that is treatable by a method, compound or pharmaceutical composition of the disclosure.

The term “in need of prevention” as used herein refers to a judgment made by a caregiver that a patient requires or will benefit from prevention. This judgment is made based on a variety of factors that are in the realm of a caregiver's expertise, but that includes the knowledge that the patient will be ill or may become ill, as the result of a condition that is preventable by a method, compound or pharmaceutical composition of the disclosure.

The term “individual”, “subject” or “patient” as used herein refers to any animal, including mammals, such as mice, rats, other rodents, rabbits, dogs, cats, swine, cattle, sheep, horses, or primates, and humans. The term may specify male or female or both, or exclude male or female or both.

The term “therapeutically effective amount” as used herein refers to an amount of a compound, either alone or as a part of a pharmaceutical composition, that is capable of having any detectable, positive effect on any symptom, aspect, or characteristics of a disease state or condition. Such effect need not be absolute to be beneficial.

The term “inhibits arrhythmia” as used herein refers to any property of a substance that tends to reduce the likelihood, severity, or duration of arrhythmia in a cardiac muscle or a heart. The muscle or heart may be part of an intact animal or may be isolated from the animal. An agent that inhibits arrhythmia may do so in the any context, including but not limited to a course of treatment or prevention.

The terms “expose” and “exposing” as used herein refers to contacting an agent to a target or causing the agent to come into contact with the target, actively or passively. The target can be living or non-living, including but not limited to organisms, organ systems, organs, tissues, cells, cell fractions, membranes, organelles, macromolecular assemblies, proteins, polypeptides, nucleic acids, cofactors, and chromosomes.

The term “arrhythmia” as used herein refers to cardiac electromechanical activities that occur independently of normal rhythmic heart function. Arrhythmia may occur in cardiac muscle, in any portion of a heart, or in an entire heart.

The term “spontaneous mechanical activity” (abbreviated SMA) as used herein refers to numerous abnormal automatic electrical and mechanical events in a muscle or myocyte occurring in the absence of pacing stimulus or despite the presence of pacing stimulus. SMA includes both sporadic and tachycardic (including STA) electrical and mechanical events.

The term “bcl-2” as used herein refers to the protein encoded by the “B-cell lymphoma 2” gene.

The term “suppressor of spontaneous mechanical activity” as used herein refers to any agent that tends to arrest, abbreviate, curtail, inhibit, reduce in severity, reduce in likelihood, reduce in duration, prevent, or in any way improve spontaneous abnormal automatic electrical and mechanical activity in a muscle or a myocyte.

The term “effective inhibitory concentration” when used herein with regard to inhibitors of SMA refers to a concentration sufficient to reduce the likelihood, severity, or duration of SMA in a myocyte or a muscle. Depending on the application, the inhibition may take the form of the prevention of SMA, or, if the inhibitory agent is introduced to the muscle or myocyte after the onset of SMA, the inhibition may take the form of the reversal of SMA.

### B. METHODS OF IDENTIFYING AN ANTI-ARRHYTHMIC AGENT

There is a lack of simple model systems capable of producing arrhythmic activity and/or SMA. This lack of simple models limits the ability to test novel pharmaceuticals for their ability to inhibit or prevent arrhythmia and SMA. Previously, all models of cardiac arrhythmia required either prolonged, artificial rapid pacing or costly animal surgery and handling to elicit arrhythmic activity. Previous approaches required treatment periods of two months to one year for in vivo arrhythmic activity to develop. This activity is not uniform across test animals and generally requires artificial stimulation for its induction.

The present disclosure provides such a novel model system for the identification of novel anti-arrhythmic agents and agents that inhibit SMA. The method described herein allows the identification of novel anti-arrhythmic agents in a controlled setting under experimentally reproducible conditions. Certain of the embodiments of the method described herein have the advantages of not requiring lengthy live animal testing, not requiring a period ranging from months to a year to complete, not requiring prolonged artificial pacing, and providing superior uniformity.

The present disclosure also provides a model system for arrhythmia that simulates sustained forms of cardiac arrhythmia, including, but not limited to, tachycardia and fibrillation as well as model systems that simulate SMA, including its sporadic and tachycardic forms. It has unexpectedly been discovered that exposure of cardiac muscle to certain agents results in the appearance of the sustained forms of automatic cardiac arrhythmia and SMA. The present disclosure further provides a model system for arrhythmia that simulates either triggered ectopy or automatic ectopy, but not both simultaneously. It has unexpectedly been discovered that exposing cardiac muscle to certain agents under certain conditions causes triggered or automatic ectopic events. Consequently, the model systems disclosed allow for the rapid screening of potential agents that inhibit or prevent tachycardia, fibrillation, other sustained forms of arrhythmia and triggered or automatic ectopy as well as agents that inhibit or prevent SMA.

The nature of this disclosure is of a model system which, under appropriate experimental conditions, can produce arrhythmic activity in atrial and ventricular muscle, including, but not limited to, disorganized, fibrillation-like arrhythmic activity and SMA. One general utility of the system described is the easy and rapid testing of pharmaceutical agents for the treatment and prevention of arrhythmia and SMA.

The utility of embodiments of the method includes, but is not limited to, drug development programs. One object of certain embodiments of this model is the identification of novel agents for the treatment and prevention of arrhythmia and SMA in a controlled setting under experimentally reproducible conditions. Such novel agents may be used to treat and/or prevent a disease state or condition associated with or characterized by arrhythmia, SMA as well as by increased bcl-2 or bcl-2 target activity. Such disease states and conditions, include, but are limited to, all types of arrhythmia known in the art and those described herein.

1. Cardiac Muscle Assay for Arrhythmia

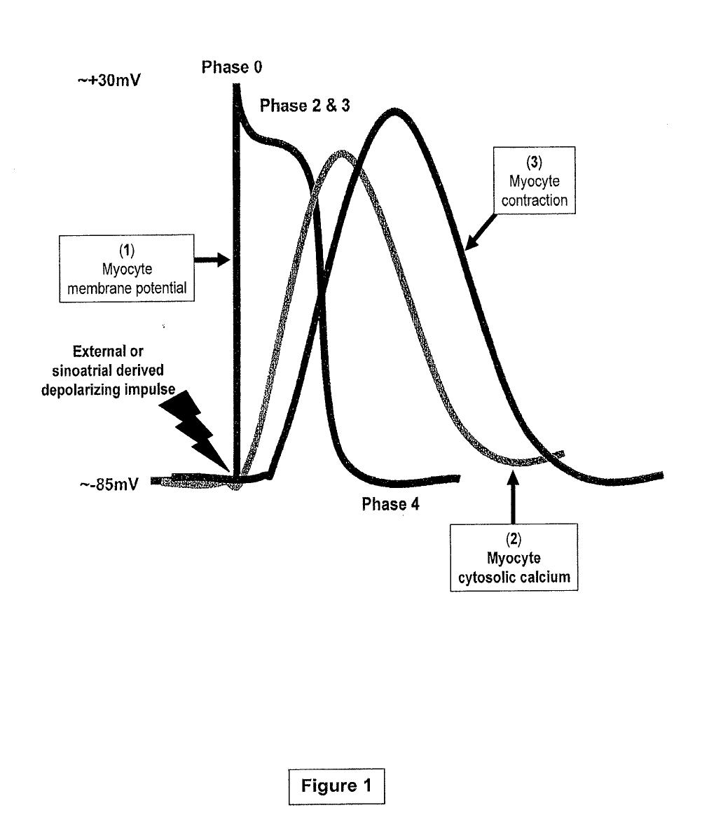

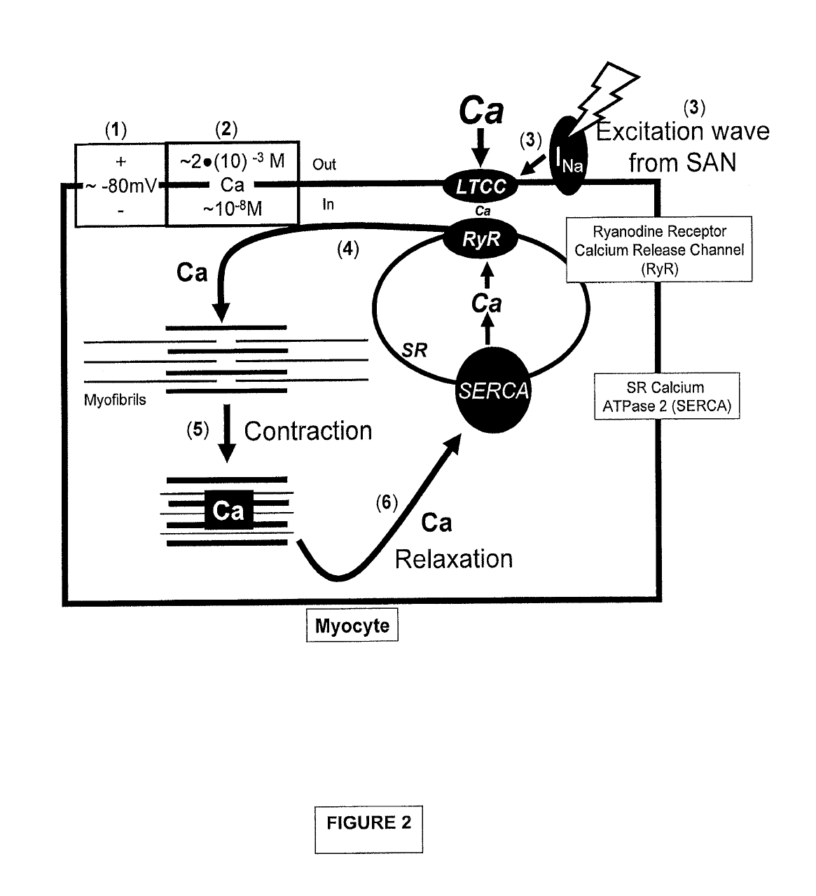

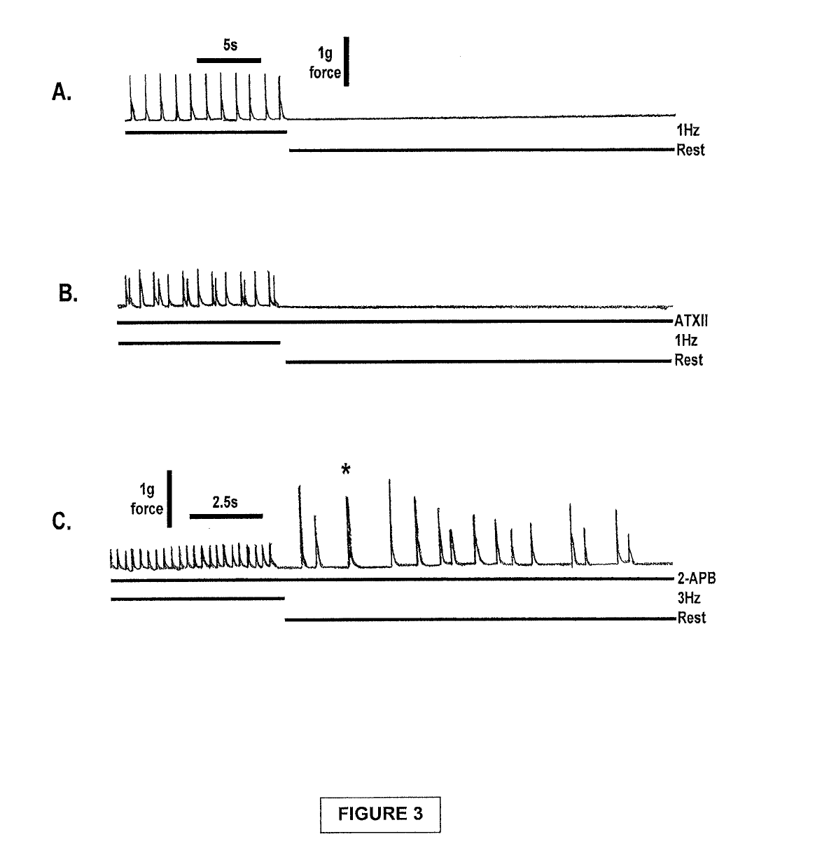

It has been unexpectedly discovered that a simple, rapid assay can be used to evaluate agents that may treat and/or prevent arrhythmia and SMA. The operation of the assay is based upon the unexpected discovery that exposing isolated rat cardiac muscle to agents that modify voltage-independent or store-operated channel (SOC) calcium homeostasis induces sporadic ectopic electrical and mechanical events under conditions of normal calcium loading (Wolkowicz et al., J. Cardiovascular Pharmacology 49:325-335 (2007); FIGS. 1A & 1B; FIGS. 2A & 2B). Exemplary agents include, but are not limited to, 2-aminoethoxydiphenyl borate (2-APB) and molecules structurally or functionally-related to 2-APB, such as for example diphenyl boronic anhydride. However, any molecule that modifies voltage-independent or store-operated channel (SOC) calcium homeostasis in a manner similar to 2-APB will effect SMA and arrhythmia, and can be used in certain embodiments of the assay. It was further unexpectedly discovered that, under conditions of increased calcium loading, such compounds as described above provoke ectopic tachycardic activity in normal rat left atrial appendage, rat left ventricular papillary muscles, and right ventricular muscle strips (Wolkowicz et al. Eur J. Pharm. 576:121-131 (2007); FIGS. 1C & 2C). Some of the embodiments disclosed here use isolated atrial and ventricular muscle, but the assay can be used in whole organs or intact animals as well.

An isolated electro-mechanical event that occurs in non-automatic heart muscle independently of stimulation is an “ectopic event” that can lead to cardiac arrhythmia. Ectopic arrhythmic activity can arise when myocytes engage in spontaneous mechanical activity (SMA) provoked by spontaneous myocyte depolarizations. Types of arrhythmia caused by SMA include but are not limited to premature contractions, tachycardia, and fibrillation.

Embodiments of the methods disclosed involve exposing viable cardiac muscle to a putative arrhythmic agent under conditions in which the cardiac muscle would otherwise display normal electro-mechanical activity.

In a general embodiment, the assay comprises the steps of: exposing a myocyte to an arrhythmic agent at an arrhythmia-inducing effective concentration, exposing the myocyte to a candidate anti-arrhythmic agent, determining a parameter indicative of arrhythmia in the myocyte in the presence of the arrhythmic agent and in the presence of the arrhythmic agent and the candidate anti-arrhythmic agent, and comparing the parameter indicative of arrhythmia in the myocyte determined in the presence of the arrhythmic agent and in the presence of the arrhythmic agent and the candidate anti-arrhythmic agent, wherein an improvement in the parameter indicative of arrhythmia determined in the presence of the arrhythmic agent and the anti-arrhythmic agent indicates the identification of an anti-arrhythmic agent.

In another embodiment, the assay may further comprise exposing the myocyte to a calcium-loading compound at a concentration effective to increase the intracellular calcium concentration of the myocyte.

In certain embodiment, the myocyte is an isolated myocyte. In other embodiments, the myocyte is a part of a cardiac muscle. The cardiac muscle may be an isolated cardiac muscle or a component of a heart (either in vivo or in vitro, such as a perfused heart). The parameter indicative of arrhythmia may be any parameter known to one of ordinary skill in the art to be associates with arrhythmia. Any parameter discussed or used herein may also be used. In one embodiment, the parameter is mechanical and/or electrical activity characteristic of arrhythmia. In an alternate embodiment, the parameter is observing an occurrence of SMA or arrhythmia, such as, but not limited to, premature contractions, tachycardia, and fibrillation. The parameter may vary depending on the model system used (as disclosed herein) and the type of arrhythmia or SMA induced. One of ordinary skill in the art is capable of identifying such parameters and understanding when such parameter is improved. Furthermore in certain embodiment, the anti-arrhythmic agent may be added prior to the arrhythmia inducing agent. The foregoing applies to the general embodiments, as well as the embodiments described below.

In one embodiment, the assay comprises the steps of: superfusing or perfusing an isolated cardiac muscle with a physiologically suitable solution, introducing pacing stimulus to the isolated cardiac muscle, measuring the rate of mechanical and/or electrical activity in the isolated cardiac muscle prior to exposure to an arrhythmia inducing agents (control condition), exposing the isolated cardiac muscle to a concentration of an arrhythmia inducing agent, such as, but not limited to, 2-APB, measuring the rate of mechanical and/or electrical activity in the isolated cardiac muscle (arrhythmic condition), exposing the isolated cardiac muscle to a concentration of a candidate anti-arrhythmia agent, and measuring mechanical and/or electrical activity of the isolated cardiac muscle (treated condition). The isolated cardiac muscle may be a non-automatic cardiac muscle. If the measurements of the treated condition are changed in the direction of the control condition, the candidate anti-arrhythmic agent is deemed to have anti-arrhythmic activity. Likewise, if the measurements of the treated condition are changed in a direction away from the arrhythmic condition, the candidate anti-arrhythmic agent is deemed to have anti-arrhythmic activity.

In another embodiment, the method comprises the steps of: superfusing or perfusing an isolated cardiac muscle with a physiologically suitable solution, introducing pacing stimulus to the isolated cardiac muscle, measuring the rate of mechanical and/or electrical activity in the isolated cardiac muscle prior to exposure to an arrhythmia inducing agents (control condition), exposing the isolated cardiac muscle to a concentration of an arrhythmia inducing agent, such as, but not limited to, 2-APB, removing pacing stimulus from the isolated cardiac muscle, measuring the rate of mechanical and/or electrical activity in the isolated cardiac muscle (arrhythmic condition), exposing the isolated cardiac muscle to a concentration of a candidate anti-arrhythmia agent, and measuring mechanical and/or electrical activity of the isolated cardiac muscle (treated condition). The isolated cardiac muscle may be a non-automatic cardiac muscle. If the measurements of the treated condition are changed in the direction of the control condition, the candidate anti-arrhythmic agent is deemed to have anti-arrhythmic activity. Likewise, if the measurements of the treated condition are changed in a direction away from the arrhythmic condition, the candidate anti-arrhythmic agent is deemed to have anti-arrhythmic activity.

In still a further embodiment, the method comprises the steps of: (a) superfusing or perfusing an isolated cardiac muscle with a physiologically suitable solution, (b) introducing pacing stimulus at an approximately sub-physiological rate to the isolated cardiac muscle, (c) measuring the rate of mechanical and/or electrical activity in the isolated cardiac muscle prior to exposure to an arrhythmia inducing agents or observing an occurrence of SMA or arrhythmia, such as, but not limited to, premature contractions, tachycardia, and fibrillation (control condition), (d) exposing the isolated cardiac muscle to a concentration of an arrhythmia inducing agent, such as, but not limited to, 2-APB, (e) exposing the isolated cardiac muscle to a concentration of an agent that increases intracellular calcium either prior to step (b) or following step (f), (f) measuring the rate of mechanical and/or electrical activity in the isolated cardiac muscle or observing an occurrence of SMA or arrhythmia, such as, but not limited to, premature contractions, tachycardia, and fibrillation (arrhythmic condition), (g) increasing pacing to an approximately physiological rate; and (h) exposing the isolated cardiac muscle to a concentration of a candidate anti-arrhythmia agent, and measuring mechanical and/or electrical activity of the isolated cardiac muscle (treated condition). The isolated cardiac muscle may be a non-automatic cardiac muscle. If the measurements of the treated condition are changed in the direction of the control condition, the candidate anti-arrhythmic agent is deemed to have anti-arrhythmic activity. Likewise, if the measurements of the treated condition are changed in a direction away from the arrhythmic condition, the candidate anti-arrhythmic agent is deemed to have anti-arrhythmic activity.

In further embodiments of the method, the isolated cardiac muscle is a left atrial appendage. In further embodiments of the method, the isolated cardiac muscle is a right ventricular muscle strip. In still a further embodiment, the isolated cardiac muscle is a ventricular strip. Other embodiments include perfused whole hearts, papillary muscles, isolated myocytes or other muscle preparations obtained either from genetically normal animals or from animals genetically modified to contain altered content or function of bcl-2 or a peptide or protein that regulates or is regulated by bcl-2 (a bcl-2 target) or that otherwise affects activity of the SOC, the IP3R or the persistent sodium channel. A bcl-2 target as used herein refers to any molecule regulated by bcl-2, either directly or indirectly, including component parts of such molecule; in specific embodiment, a bcl-2 target is the IP3R, the SOC and/or the cardiac persistent sodium channel. In further embodiments of the method, the physiologically suitable solution comprises Krebs-Henseleit (KH) perfusate but it can also be whole blood or isolated blood cells; the physiologically suitable solution may be at body temperature or below body temperature. In further embodiments, the pacing stimulus is used at physiological rates or rates lower or higher than physiological rates.

In further embodiments of the method, the method further comprises inducing high concentrations of intracellular calcium. In further embodiments, increasing intracellular calcium further comprises contacting the muscle with a substance capable of inducing an increase in intracellular calcium. Exemplary agents include, but not limited to, activators of β-adrenergic signaling, activators of cyclic AMP (cAMP) signaling, slow calcium channel activators, ouabain, agents that prolong action potential duration or alter sodium channel activity, genetic manipulations that affect action potential duration, sodium channel activity, and intracellular calcium and a combination thereof. For example, the agent capable of inducing an increase in intracellular calcium is one of the following: isoproterenol, forskolin, BayK 8644, FPL-64176, ATX II, clofilium+a Gαq-coupled agonist, angiotensinII, genetic manipulations that affect action potential duration and intracellular calcium and/or ouabain. In further embodiments, the concentration of 2-APB or a related compound is sufficient to induce SMA or arrhythmia in the cardiac muscle.

In further embodiments the muscle is exposed to the arrhythmia-inducing agent at a concentration at or above about 20 μM. In further embodiments the arrhythmia-inducing agent is 2-APB, a derivative of 2-APB, a structurally-related compound, a functionally related compound, or a combination thereof. In further embodiments, these arrhythmia-inducing agents may be chemically modified with groups such as but not limited to azido or iodine moieties in order to create the potential for in vivo or in vitro covalent attachment of the arrhythmic agent to the intracellular proteins that control arrhythmia including but not limited to bcl-2 or a bcl-2 target. Such an approach would allow for the subsequent activation of these modified arrhythmic agents to produce a stable, ectopic focus in isolated or intact heart muscle or myocytes from normal or genetically modified animals. In further embodiments, the suspected anti-arrhythmic agent is a previously established anti-arrhythmic agent employed as a positive control.

Embodiments in which high levels of intracellular calcium are induced have the advantage of inducing continuous arrhythmias, such as tachycardia and fibrillation.

The cardiac muscle may be part of a functioning heart (in vivo or in vitro), but it in certain embodiments is isolated from a host animal. The muscle may be from any mammal, but it is preferably from a commonly used animal model or from a human. Commonly used animal models that can provide suitable cardiac muscle include but are not limited to Norway rat, cotton rat, mouse, cavy, cat, hamster, dog, gerbil, sheep, goat, rabbit, swine, monkey, or ape. If the animal source of the muscle is a commonly used animal model, it can be of any subspecies or breed. Such animals can be obtained from sources familiar to those skilled in the art. The animal source may be genetically modified or unmodified, according to the needs of the specific assay.

Isolated cardiac muscle can be removed from live or recently dead mammalian subjects by methods familiar to those skilled in the art. Preferably, the handling of the animals will conform to the standards set out in the Guide for the Care and Use of Laboratory Animals published by the U.S. National Institutes of Health (NIH Pub. No. 85-23, 1996).

In embodiments of the method, the isolated cardiac muscle is left atrial appendage (LAA), ventricular muscle strip or right ventricular muscle strip (RVMS) isolated from the animal. However, other cardiac muscles may be used. The left atrium and the ventricles have the advantage of having no automatic activity. The automatic activity of the right atrium, for example, could potentially interfere with observations of arrhythmia in the muscle. Such interference will not be present when left atrium or left ventricle is used. The LAA and RVMS have the additional advantage of being well understood systems and of requiring far less media than is required by other perfused muscle systems. This latter is an even greater advantage if costly agents are the subject of the assay, as large amounts of the agent will be required to maintain a steady concentration in large volumes of perfusate. If it is desired to study the effects of an agent on automatic activity, then the right atrium would then be preferable. Studies using isolated myocytes are another embodiment with the advantage of allowing the molecular or genetic manipulation of potential anti-arrhythmic target proteins such as bcl-2 or bcl-2 targets.

Cardiac muscle can be prepared by any method known by those skilled in the art. For example, animals can be injected with appropriate doses of anesthetics to achieve a deep plane of sedation. An appropriate dose of heparin can then be administered to these animals to prevent blood coagulation during all subsequent procedures. A bilateral thoracotomy can be performed and hearts then extirpated and placed in ice-cold KH buffer. Intact hearts would then be trimmed, mounted on a perfusion cannula and then perfused in a Langendorff- or working heart-mode (one example of this approach is described in Balschi J A, et al., “Model systems for modulating the free energy of ATP hydrolysis in normoxically perfused rat hearts” J. Molecular and Cellular Cardiology 29:3123-3133 (1997), which is hereby incorporated by reference for such teaching). Alternatively, appropriate muscles are surgically isolated from intact hearts and superfused in a standard muscle bath; these isolated muscles include left atrial appendage, right ventricular muscle strips (for examples of this approach see Grupp I L, and Grupp G. “Isolated heart preparations perfused or superfused with balanced salt solutions” in Methods in Pharmacology vol. 5, Plenum Press: New York, pages 111-128 (1984), which is hereby incorporated by reference for such teaching) or right or left ventricular papillary muscles (for examples of this approach, see Urthaler et al., “Estimates of beat to beat handling of activator calcium using measurements of [Ca²⁺]ᵢ in aequorin loaded ferret cardiac muscle” Cardiovascular Research 28: 40-46 (1994), which is hereby incorporated by reference for such teaching).

If the assay is performed in vitro, the isolated muscle must be maintained in an appropriate muscle bath. An appropriate muscle bath must allow the isolated muscle to function mechanically and/or electrically during the assay. Such muscle baths include any such baths familiar to those skilled in the art. One example is KB buffer with added glucose (NaCl 118 mM, NaHCO₃ 27 mM, KCl 4.8 mM, MgSO₄ 1.2 mM, KH₂PO₄ 1.0 mM, CaCl₂ 1.8 mM, glucose 11.1 mM). The buffer must be at a suitable temperate, appropriate to allow functioning of the muscle, such as in one embodiment at about physiological temperature (about 37° C.). The assay may be performed at a sub-physiological temperature (such as 30° C.); as described below, the ability of 2-APB to induce tachycardia under proper conditions is greater at 37° C. than at 30° C. The isolated muscle may be superfused with the buffer prior to the assay at an appropriate temperature, for example about 30° C.

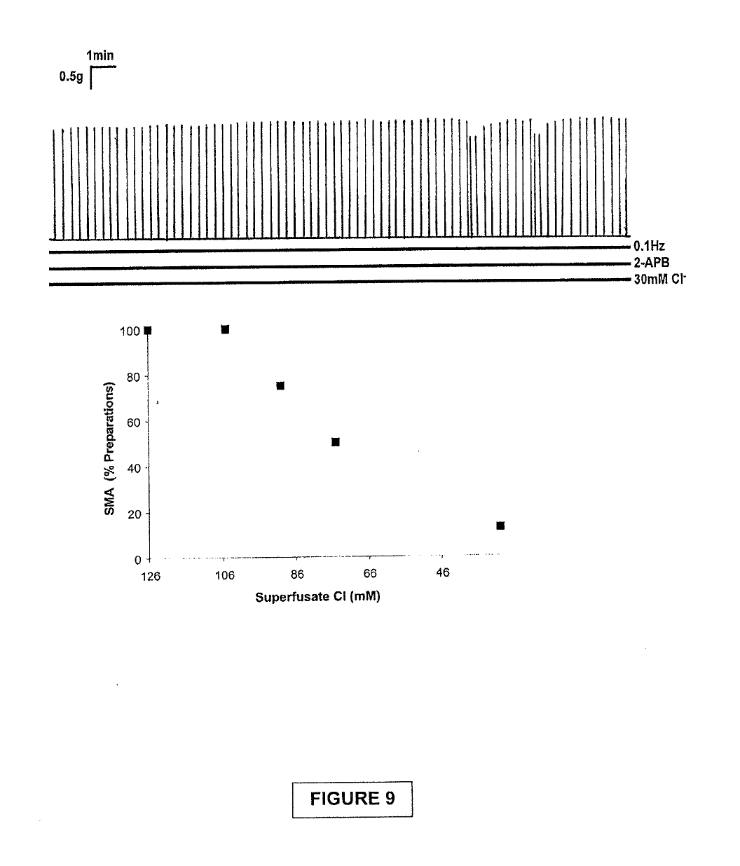

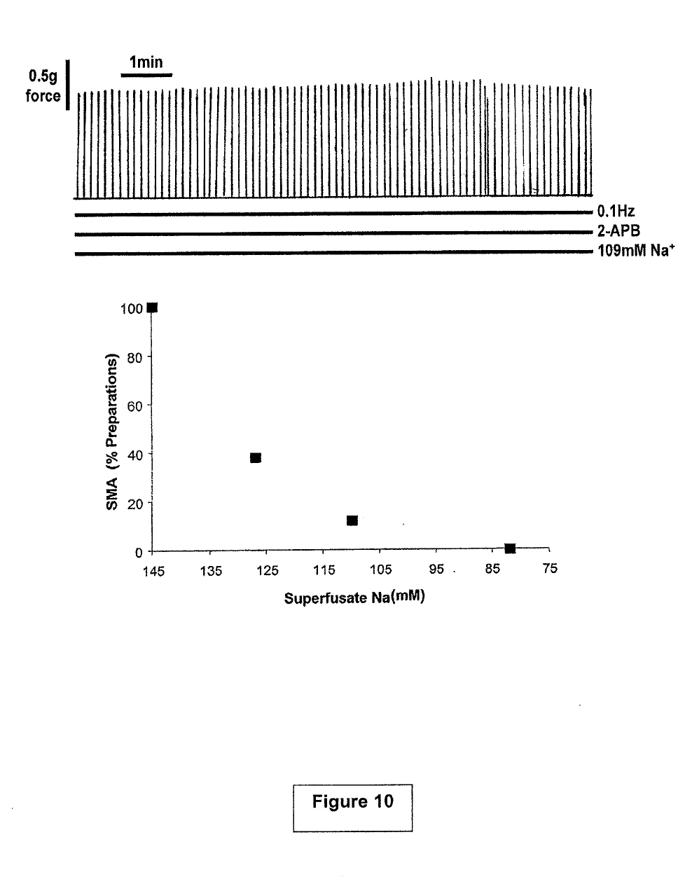

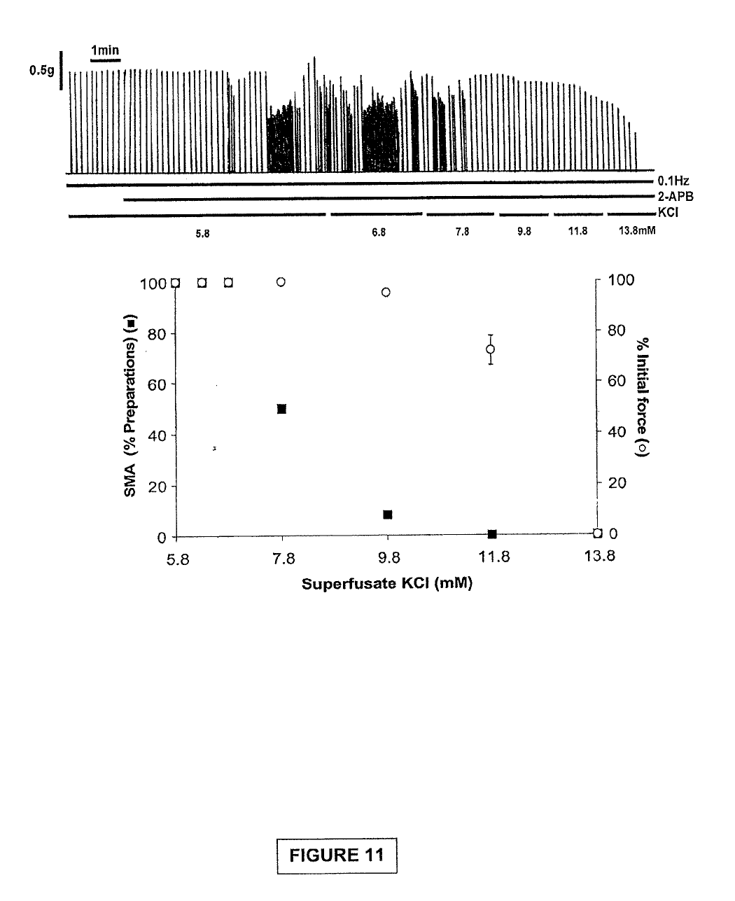

Suitable muscle bath or buffer preferably contains adequate concentrations of potassium, sodium and chloride to permit 2-APB to act as an arrhythmia inducing agent. As an example, in KH buffer with added glucose, it has been observed that the ability of 2-APB to induce arrhythmia starts to decrease below around 106 mM chloride (FIG. 9). In KH buffer with added glucose, it has been observed that the ability of 2-APB to induce arrhythmia also starts to decrease below around 145 mM sodium (FIG. 10), and disappears at about 80 mM sodium. In KH buffer with added glucose, it has been observed that the ability of 2-APB to induce arrhythmia drops when the concentration of potassium is above about 6 mM and disappears when the concentration of potassium reaches about 11.8 mM (FIG. 11).

In alternative embodiments, the cardiac muscle is a component of the heart of a live animal. The steps of the assay can be carried out in live animals by means familiar to those skilled in the art.

In one embodiment, animals are anesthetized and intubated, a pressure transducer is inserted into the femoral or other appropriate artery, a lead II ECG is obtained, and their intact beating hearts are exposed (see, for example, Huang J. et al., “Restitution properties during ventricular fibrillation in the in situ swine heart” Circulation 110:3161-3167 (2004), which is hereby incorporated by reference for such teaching). Piezoelectric crystals then may be implanted into the ventricle to acquire local mechanical activity in these intact hearts (see, for example, Wolkowicz et al., “Sodium-calcium exchange in dog heart mitochondria: effects of ischemia and verapamil” American Journal of Physiology 244: H644-H651 (1983), which is hereby incorporated by reference for such teaching). Appropriate electrical measurements may also be obtained using glass micro-electrodes, plunge needle electrodes or other electrical data recording devices (see, for example, Huang J. et al., “Restitution properties during ventricular fibrillation in the in situ swine heart” Circulation 110:3161-3167 (2004), which is hereby incorporated by reference for such teaching).

Five instructive exemplars are given for the administration of 2-APB and functionally related compounds to these beating in vivo hearts.

First, a solution of concentrated (˜1M) 2-APB or functionally related compound suspended in DMSO may be placed directly onto the intact left atrium, left ventricle or other region of the beating heart as is experimentally desired. This can be accomplished using various approaches including a cotton swab, a cotton pad or using a dispenser such as a pipetter. Second, a solution of concentrated 2-APB or functionally related compound could be placed into an appropriate syringe and this solution can be injected directly into the ventricular epi-, mid- or endo-myocardium or into the atrial muscle wall. The needle used should be of an appropriate gauge so as not to compromise muscle integrity (see Scherf D., “Studies on auricular tachycardia caused by aconitine administration.” Proceedings of the Society for Experimental Biology 64: 233-239 (1947), which is hereby incorporated by reference for such teaching). Third, an experimentally important artery may be isolated and cannulated, and the perfused heart bed may then be infused with saline or blood solutions containing appropriate concentrations of 2-APB or functionally related molecules. These functionally related compounds may be modified with moieties that permit the covalent attachment of these compounds to the intracellular proteins that control arrhythmia including but not limited to bcl-2, the IP3R and the SOC. Subsequent activation of these compounds would covalently link them to the target arrhythmic protein including but not limited to bcl-2, the IP3R or the SOC.

Using these approaches, a stable focus of sporadic ectopic activity should be produced. Changes in in vivo heart electrical activity can be monitored through a lead II ECG and the appearance of ectopic depolarizations is the end point for this approach. Likewise, local or whole organ electro-mechanical activity can be measured using the pressure transducer, microelectrode recording devices and/or the piezoelectric crystals (for example, see Wolkowicz et al., “Sodium-calcium exchange in dog heart mitochondria: effects of ischemia and verapamil” American Journal of Physiology 244: H644-H651 (1983), and Huang J. et al., “Restitution properties during ventricular fibrillation in the in situ swine heart” Circulation 110:3161-3167 (2004), which are hereby incorporated by reference for such teaching). The sporadic, ectopic electro-mechanical events will be either atrial or ventricular in origin depending on the point of 2-APB application.

Fourth, intact animals could be infused with appropriate concentrations of isoproterenol, ouabain, ATX-II, or related compounds that induce heart muscle calcium loading or their sympathetic and/or parasympathetic systems may be modulated to increase or decrease cell calcium. Application of 2-APB or a functionally related compound to these calcium-loaded hearts using any of the three methods noted above should affect an arrhythmic tachycardia. Fifth, the blood vessel perfusing the area to which 2-APB had been applied or administered could be cannulated, and this region could be selectively infused with a calcium loading agent to induce a tachycardic focus in this region of the heart.

The in vivo efficacy of anti-arrhythmic agents can then be tested by methods understood by those skilled in the art. The following three general methods for testing can be used by way of example. First, the test anti-arrhythmic compounds could be infused into the general circulation. Second, the test anti-arrhythmic compounds could be infused through a cannula into the specific muscle bed that had been made ectopic. Third, animals could be pre-treated with a test anti-arrhythmic agent in their drinking water or chow prior to the induction of sporadic or tachycardic arrhythmias. Under these in vivo conditions a candidate anti-arrhythmic agent is identified if the test agent suppresses sporadic or tachycardic ectopic activity and restores or maintains lead II ECG characteristics to normal or in the direction of normal.

In embodiments of the assay the muscle must be paced by any appropriate means familiar to those skilled in the art. If the muscle is intact in a living subject, or if the muscle includes a sinoatrial node, pacing may be provided by the sinoatrial node, or it may be provided externally (such as by a pacemaker). If the muscle is an isolated cardiac muscle, then some form of external pacing must be provided, including but not limited to the example of a pace-making device. The pacing rate may be approximately physiological, supra- or sub-physiological. The pacing rate may be greater than approximately physiological; however, if the assay is to detect an agent with properties of inducing tachycardia, then it is preferable that the pacing rate not significantly exceed an approximately physiological rate. Physiological rates of pacing vary between species, and roughly correspond to the animal's heart rate. Physiological pacing rates of different species of mammal are well understood by those skilled in the art. For example, the range of about 5-6 Hz falls within about the physiological range for a Norway rat. The approximate heart rate of a healthy Norway rat is 330-480 beats/minute. The approximate heart rate of a healthy mouse is about 632+/−51 beats per minute (adult) or 286+/−56 beats per minute (newborn). The approximate heart rate of a healthy cavy is 240-300 beats per minute. The approximate heart rate of a healthy gerbil is 360 beats per minute. The approximate heart rate of a healthy hamster is 250-500 beats per minute. The approximate heart rate of a healthy Rhesus monkey is 120-180 beats per minute. The approximate heart rate of a healthy sheep or goat is 80-120 beats per minute. An animal's heart rate may vary greatly depending on its sex, age, state of activity, temperature, culture conditions (in vitro) or state of health, as is understood by those skilled in the art.

The rate of pacing may be varied according to the needs of the assay. In some embodiments, the muscle is paced at the same rate throughout. In some embodiments, the rate of pacing may be varied at certain points during the assay. In some embodiments, including but not limited to assays for agents that inhibit tachycardia, initial pacing occurs at approximately a sub-physiological rate, and the pacing rate is increased to an approximately physiological rate. In assays for tachycardia, the pacing rate may be increased after the appearance of tachycardia.

In embodiments of the assay an agent to induce arrhythmia will be introduced in the muscle. The arrhythmic agent may be chemical or physical. If the agent is chemical, it can be any chemical agent known by those skilled in the art to induce arrhythmia. The chemical agent will be introduced at a concentration sufficient to induce arrhythmia. If a certain type of arrhythmia is the subject of the assay (for example, triggered activity, tachycardia or any other type of arrhythmia), then the agent should be present at a concentration sufficient to produce the certain type of arrhythmia. One acceptable agent is anemone toxin II (“ATX II,”), the toxin of Anemone sulcata. ATX II is a sodium channel regulator. ATX II causes triggered activity in the presence but not in the absence of pacing. Low concentrations of ATXII (<50 nM) induce triggered activity while higher concentrations (>˜50 nM) provokes automatic activity.

One class of chemical arrhythmic agents is the class of modifiers of voltage-independent calcium homeostasis. One modifier of voltage-independent calcium homeostasis is 2-aminoethoxydiphenyl borate (“2-APB”), and structurally/functionally related compounds, which has been unexpectedly discovered to induce arrhythmia in myocytes and isolated cardiac muscle. Although not being limited by other mechanisms, 2-APB is used as an experimental activator of intracellular calcium leak through the inositol 1,4,5-trisphosphate receptors (“IP3R”) and voltage-independent calcium entry through the SOC. 2-APB affects cell calcium homeostasis in a concentration-dependent manner by depressing IP3R activity and SOC-linked calcium entry at low concentrations while inducing calcium leakage and SOC calcium entry in isolated myocytes and non-excitable cells at slightly higher concentrations. Such pharmacologically-induced calcium leakage and entry may be relevant in atria, as atrial myocytes contain substantial type 2 IP3R whose aberrant activation contributes to mechanical instability in these isolated cells. Electromechanical disturbances occur in isolated atrial myocytes experiencing high levels of IP3R-linked signaling. Likewise, ventricular myocytes and conduction system cells contain moderate to high levels of IP3R and are be susceptible to calcium leakage through this channel. Prior art shows that 2-APB provokes voltage-independent SOC calcium entry over the concentration range which elicits SMA in rat left atria (Peinelt C. et al. “2-Aminoethoxydiphenyl borate directly facilitates and indirectly inhibits STIM1-dependent gating of CRAC channels.” Journal of Physiology 586: 3061-3073 (2008), which is hereby incorporated by reference for such teaching). Thus without being limited to other mechanisms and without limiting the scope of the present disclosure, the effective target for 2-APB and related compounds is myocardial voltage-independent calcium signaling including but not limited to bcl-2, the IP3R and the SOC. Downstream targets include but are not limited to a cardiac persistent sodium channel. Components include but are not limited to the transient receptor potential proteins.

In embodiments of the assay 2-APB or structurally/functional related compounds will effectively induce arrhythmia at concentrations of about 15 μM and greater, or between 7.5 and 15 μM. In embodiments of the assay, the concentration of 2-APB is 20 μM or greater. In some embodiments of the method, an arrhythmic agent related to 2-APB is used at an arrhythmia-inducing effective concentration. In some embodiments the concentration may be about 15 μM or greater, from 7.5-15 μM, or greater that 20 μM. Any effective concentration may be used, as can be determined empirically by those skilled in the art.

In embodiments the concentration of intracellular calcium may be increased during the assay. Increased levels of calcium have been unexpectedly discovered to allow 2-APB or structurally/functionally related molecules to induce sustained forms of arrhythmia, such as tachycardia and fibrillation. The increase may occur at any time, including but not limited to prior to the addition of 2-APB or following the appearance of arrhythmia. The increase in intracellular calcium may be achieved by any method familiar to those skilled in the art, including but not limited to exposure of the muscle to activators of β-adrenergic signaling, activators of cAMP signaling, slow calcium channel activators, ouabain, angiotensin II, agents that prolong action potential duration or genetic changes that prolong the action potential duration, alter persistent sodium channel activity, or alter voltage-dependent/independent calcium signaling and a combination thereof. In further embodiments, the substance capable of inducing an increase in cytoplasmic calcium is one of the following non-exhaustive set of agents: isoproterenol, forskolin, BayK 8644, FPL-64176, ATX II, (clofilium+a Gαq agonist), angiotensin II and/or ouabain. Further embodiments include isolated muscles, intact heart or intact animals that contain genetic modifications that increase the action potential duration, alter persistent sodium channel activity, or modify cell calcium homeostasis.

In embodiments of the assay the mechanical or electrical activity of the muscle is measured over a period of time beginning prior to the exposure of the muscle to a possible anti-arrhythmic agent and lasting at least until after the exposure of the muscle to a possible anti-arrhythmic agent. Mechanical and/or electrical activity can be measured by any suitable method, including but not limited to use of a force transducer or an oscilloscope. Such measurement should measure the rate of mechanical or electrical activity, and may optionally measure the force or amplitude of mechanical or electrical activity.

As additional examples, two complementary approaches can be used to identify candidate anti-arrhythmic compounds in isolated cardiac myocytes. First, myocytes could be analyzed using patch clamp approaches. In particular myocyte transmembrane action potentials and currents could be recorded in calcium-tolerant myocytes using the whole-cell patch-clamp technique. Patch pipettes would be prepared from borosilicate glass and possess a resistance of 2-4MΩ when filled with pipette solution. Pipettes would be connected to the head stage of a patch clamp amplifier for example, an Axopatch 200B amplifier. After establishing the whole-cell configuration, cell membrane capacitance and series resistance would be determined using a 10 mV hyperpolarizing pulse and used to compensate all signal analyses. All current and voltage signals would be filtered at 5 KHz. Myocyte current density would be determined by dividing the measured peak current amplitude by cell capacitance using methods known to those skilled in these arts, for example Clampex 9.0 software. Action potentials would be measured using myocytes in the whole-cell current-clamp mode and superfused with Tyrodes. The pipette solution would include [in mM]: KCl [100], NaCl [10], ATP-Mg [5], EGTA [10], MgCl_{2 [}2], HEPES [10], glucose [5], GTP-Mg [0.5] @ pH 7.2 with KOH. After establishing a whole-cell configuration, the amplifier would be switched to current-clamp mode. In normal, untreated myocytes, action potentials would be elicited with a 6 ms current pulse (30% above threshold) at a 30 s interval. Resting membrane potential, the action potential amplitude, and the time to 50% (APD50) and 90% repolarization (APD90) would be recorded and analyzed using software known to those who practice these arts. Such measurements would establish baseline values for individual cells. Immediately thereafter, myocytes would be exposed to a compound sufficient to induce arrhythmia and/or SMA. In other forms of the assay, myocytes could also be exposed to agents that increase cell calcium content. In the whole-cell current-clamp mode, SMA or STA would be measured as action potentials in the absence of the 6 ms current pulse; untreated cells require this pulse to elicit an action potential. An anti-arrhythmic compound would be any agent which prevents or reverses this ectopic electrical activity and restores to myocytes their need for external stimulation in order to produce electrical activity.

In the second approach, myocyte contraction and intracellular calcium transients could be recorded simultaneously using methods known to those skilled in the art, for example, using an IonOptix fura-2 fluorescence and edge detection system. Briefly, myocytes isolated from normal animals or from genetically modified animals would be incubated with a calcium sensitive dye for example fura-2/AM and then washed with superfusate. Calcium dye-loaded myocytes then would be placed in a cell perfusion chamber mounted on an inverted microscope for detection of dye fluorescence, for example a Nikon TE 2000 microscope, and perfused with normal superfusate. Myocyte would be field stimulated using two platinum electrodes on either side of the perfusion chamber; field stimulation would elicit myocyte calcium transients and contraction. These activities would be quantitated by measuring the change in sarcomere length using methods known to those skilled in these arts. Myocyte calcium-sensitive dye would be excited using, for example, a collimated light beam from a 150-W Xe arc lamp. Changes in dye fluorescence would be recorded and used to analyze changes in myocyte cytosolic calcium; in the case of Fura-2, 340-to-380 nm fluorescence ratios would measure changes in myocyte cytosolic calcium. Signal would be restricted to one cell using techniques known to those skilled in these arts. Light signals would be digitized and subsequently analyzed. Specific experiments would follow the pattern noted for patch clamp analyses. Calcium transients and contractions would first be measured in untreated myocytes to establish baseline values. Unloaded or calcium-loaded myocytes isolated from normal or genetically modified animals then would be exposed a compound to induce arrhythmia and/or SMA. These arrhythmic events would be observed as calcium transients or contractions that occur in the absence of external stimulus from the field stimulators. An anti-arrhythmic agent would be any compound that prevents or reverses the appearance of these ectopic calcium transients and contractions.

For example, isolated superfused or perfused muscles can be impaled with conventional glass microelectrodes of 10 to 30MΩ resistance; these microelectrodes are filled with a solution of 3M potassium chloride. Microelectrodes are routinely mounted on 30 μm silver-silver chloride spiral wire to allow for freedom of motion. Following the successful impalement of an individual myocyte, action potentials can be recorded with DC coupling at the center of the ring as the difference in voltage between the intracellular microelectrode and the extracellular silver-silver chloride reference electrode. Electrical signals are passed through a high-impedance capacitance-compensation preamplifier and are recorded on a personal computer at a sampling rate of ˜3 Hz. Subsequent data analysis of the action potentials of these muscles is performed using standard software (for an example of such a protocol, see Huang J et al., “Restitution properties during ventricular fibrillation in the in situ swine heart” Circulation 110:3161-3167 (2004), which is hereby incorporated by reference for such teaching).

The electrical activity of intact hearts in surgically-prepared, open- or closed-chested animals can be obtained using a standard lead II ECG (for examples see Straeter-Knowlen I et al., “¹H NMR spectroscopic imaging of myocardial triglycerides in excised dog hearts subjected to 24 hours of coronary occlusion” Circulation 93: 1464-1470 (1996)) which is hereby incorporated by reference for such teaching). Intact heart electrical activity also can be measured using microelectrode or plaque electrode techniques known to those skilled in this art (see Huang J et al., “Restitution properties during ventricular fibrillation in the in situ swine heart” Circulation 110:3161-3167 (2004), which is hereby incorporated by reference for such teaching).

Subsequent to exposure of the muscle to the arrhythmic agent, arrhythmia will be measured in the muscle. If normal rhythmic activity is restored subsequent to exposure of the muscle to the possible anti-arrhythmic agent, or if ectopic activity is reduced in frequency or severity, then it can be concluded that the agent inhibits arrhythmia.

2. Molecular Assays

The present disclosure also provides for molecular biology methods of screening for the identification of novel anti-arrhythmic agents and agents that inhibit SMA. In certain embodiments, the molecular biology assays involve the identification of agents that bind to bcl-2 or a bcl-2 target, inhibit bcl-2 activity or expression (directly or indirectly), that inhibit the activity or expression of a bcl-2 target (directly or indirectly), that regulate the calcium leak regulated by IP3R, that suppress voltage-independent calcium entry including but not limited to that mediated by IP3R or the SOC and its components, that suppress voltage-independent calcium entry including but not limited to that mediated by ryanodine receptor and that regulate downstream targets of the IP3R and SOC including but not limited to the cardiac persistent sodium channel.

In general, such screening methods comprise the steps of providing a molecular assay system (as described in more detail below) that expresses bcl-2 or a bcl-2 target molecule, introducing into the assay system a test compound and determining whether the test compound inhibits the activity or expression of bcl-2, or the activity or expression of a bcl-2 target. Such inhibition or modulation may: (i) directly inhibit the activity of bcl-2 or a bcl-2 target (ii) indirectly inhibit bcl-2 or a bcl-2 target by affecting a molecule or polypeptide that regulates bcl-2 or a bcl-2 target, or (iii) inhibit or modulate the expression or stability of bcl-2 or a bcl-2 target.

The methods involve the identification of candidate or test compounds or agents (including but not limited to, polypeptides, functional nucleic acids, carbohydrates, antibodies, small molecules or other molecules) which (i) bind to bcl-2 or a bcl-2 target, (ii) regulate directly or indirectly bcl-2 or a bcl-2 target, or (iii) have an inhibitory effect on the activity, the expression and/or the stability of bcl-2 or a bcl-2 target. Such compounds may then be further tested in appropriate systems (such as, but not limited to, the animal models systems described herein) to determine the activity of the identified compounds.

Candidate compounds are identified using a variety of assays, such as, but not limited to, assays that employ cells which express bcl-2 or a bcl-2 target (cell-based assays) or in assays with isolated bcl-2 or a bcl-2 target (cell-free assays). The various assays can employ a variety of variants of bcl-2 and bcl-2 targets (e.g., full-length, a biologically active fragment, or a fusion protein which includes all or a portion of the desired polypeptide). Moreover, bcl-2 or a bcl-2 target can be derived from any suitable bacterial species, mammalian species or mammalian model organism (e.g., human, rat or murine).

Where the assay involves the use of a whole cell, the cell may either naturally express bcl-2 or a bcl-2 target or may be modified to express the same. In the latter case, cells can be modified to express such polypeptides through conventional molecular biology techniques, such as by infecting the cell with a virus comprising a nucleic acid sequence coding for such polypeptide such that the polypeptide is expressed in the cell following infection. The cell can also be a prokaryotic or a eukaryotic cell that has been transfected with a nucleic acid sequence encoding such polypeptides. In the foregoing, variety of variants of bcl-2 and bcl-2 targets may be used, such as a full-length polypeptide, a biologically active fragment, or a fusion protein which includes all or a portion of the desired polypeptide.

The assay can be a binding assay entailing direct or indirect measurement of the binding of a test compound to bcl-2 or a bcl-2 target. The assay can also be an activity assay entailing direct or indirect measurement of the activity of bcl-2 or a bcl-2 target. The assay can also be an expression assay entailing direct or indirect measurement of the expression of bcl-2 or a bcl-2 target. The assay can also be a stability assay entailing direct or indirect measurement of the stability of mRNA or protein. The mRNA may be but is not limited to bcl-2 mRNA or the mRNA of a bcl-2 target.

The various screening assays may be combined with an in vivo assay entailing measuring the effect of the test compound on the symptoms of the disease states and conditions discussed herein. In such an embodiment, the compounds may be evaluated to determine if they impact a parameter associated with the action of bcl-2 or a bcl-2 target. Such parameters include, but are not limited to, determining whether the subject shows signs of arrhythmia or SMA.

Therefore, in one embodiment the assay for the identification of an anti-arrhythmic agent comprises the steps of: contacting a polypeptide with a candidate anti-arrhythmic agent, wherein the polypeptide is selected from the group consisting of bcl-2 and a bcl-2 target and measuring the binding between the polypeptide and the candidate anti-arrhythmic agent, wherein binding between the polypeptide and the candidate anti-arrhythmic agent indicates the identification of an anti-arrhythmic agent.

In another embodiment the assay for the identification of an anti-arrhythmic agent comprises the steps of: contacting a cell that expresses a polypeptide selected from the group consisting of bcl-2 and a bcl-2 target with a candidate anti-arrhythmic agent and measuring the activity of the polypeptide in the presence of and the absence of the candidate anti-arrhythmic agent, wherein a decrease in activity of the polypeptide in the presence of the anti-arrhythmic agent indicates the identification of an anti-arrhythmic agent.

In yet another embodiment the assay for the identification of an anti-arrhythmic agent comprises the steps of: contacting a cell that expresses a polypeptide selected from the group consisting of bcl-2 and a bcl-2 target with a candidate anti-arrhythmic agent and measuring the expression or stability of the polypeptide in the presence of and the absence of the candidate anti-arrhythmic agent, wherein a decrease in expression or stability of the polypeptide in the presence of the anti-arrhythmic agent indicates the identification of an anti-arrhythmic agent.

In a further embodiment of the assays described above, the bcl-2 target may an IP3R, a SOC, a persistent sodium channel, or a combination of the foregoing.

a. Binding of bcl-2 or bcl-2 Targets

In one embodiment, the present disclosure provides assays for screening candidate or test compounds which bind to or modulate the activity of bcl-2 or a bcl-2 target. Such polypeptides may be expressed by a cell, membrane bound or contained in a liposome, micelle or similar lipid containing structure. Such assays can employ a variety of variants of bcl-2 and bcl-2 targets, such as a full-length polypeptide, a biologically active fragment, or a fusion protein which includes all or a portion of the desired polypeptide. As described in greater detail below, the test compound can be obtained by any suitable means (such as from conventional compound libraries).

Determining the ability of the test compound to bind to bcl-2 or a bcl-2 target can be accomplished, for example, by coupling the test compound with a radioisotope or enzymatic label such that binding of the test compound to bcl-2 or a bcl-2 target can be measured by detecting the labeled compound in a complex. For example, the test compound can be labeled with ¹²⁵I, ³⁵S, ¹⁴C, or ³H, either directly or indirectly, and the radioisotope detected by direct counting of radio-emission or by scintillation counting. Alternatively, the test compound can be enzymatically labeled with, for example, horseradish peroxidase, alkaline phosphatase, or luciferase, and the enzymatic label detected by determination of conversion of an appropriate substrate to product.

In a competitive binding format, the assay comprises contacting bcl-2 or a bcl-2 target with a known compound which binds to bcl-2 or a bcl-2 target, contacting the assay mixture with a test compound, and determining the ability of the test compound to preferentially bind bcl-2 or a bcl-2 target as compared to the known compound. Such polypeptides may be expressed by a cell, membrane bound or contained in a liposome, micelle or similar lipid containing structure.

b. Inhibition of Signaling

In another embodiment, the assay is a cell-based assay comprising contacting a cell expressing bcl-2 or a bcl-2 target with a test compound and determining the ability of the test compound to inhibit the activity of bcl-2 or the bcl-2 target. Such assays can employ a variety of variants of bcl-2 and bcl-2 targets, such as a full-length polypeptide, a biologically active fragment, or a fusion protein which includes all or a portion of the desired polypeptide. Such polypeptides may be expressed by a cell, membrane bound or contained in a liposome, micelle or similar lipid containing structure. Determining the ability of the test compound to inhibit the activity of these proteins can be accomplished by any method suitable for measuring the activity of these proteins or the activity of a G-protein coupled receptor or other seven-transmembrane receptor. The activity of a seven-transmembrane receptor can be measured in a number of ways, not all of which are suitable for any given receptor. Among the measures of activity are: alteration in intracellular Ca²⁺ concentration, activation of phospholipase C, alteration in intracellular inositol triphosphate (IP3) concentration, alteration in intracellular diacylglycerol (DAG) concentration, and alteration in intracellular adenosine cyclic 3′,5′-monophosphate (cAMP) concentration. In one embodiment, determining the ability of the test compound to modulate the activity of bcl-2 or a bcl-2 target can be accomplished by determining the ability of bcl-2 to bind to or interact with a bcl-2 target. The bcl-2 target can be any molecule with which bcl-2 binds or interacts with in nature including but not limited to the IP3R and SOC. The target molecule can be a component of a signal transduction pathway which facilitates transduction of an extracellular signal generated by bcl-2 or in which bcl-2 participates. The bcl-2 target in such case can be, for example, a second intracellular protein which has catalytic activity or a protein which facilitates the association of downstream signaling molecules with bcl-2. This target molecule may be the IP3R, the SOC or another myocyte cell protein including but not limited to a cardiac persistent sodium channel.

Determining the ability of bcl-2 to bind to or interact with a bcl-2 target can be accomplished by one of the methods described above for determining direct binding. In one embodiment, determining the ability of a compound of the invention to bind to or interact with a target molecule can be accomplished by determining the activity of the bcl-2 target. For example, the activity of the bcl-2 target can be determined by detecting induction of a cellular second messenger of the target (intracellular Ca²⁺, diacylglycerol, IP₃, etc.), detecting catalytic/enzymatic activity of the bcl-2 target on an appropriate substrate, detecting the induction of a reporter gene (such as a regulatory element that is responsive to a compound operably linked to a nucleic acid encoding a detectable marker, e.g., luciferase), detecting a cellular response or detecting the effect of such bcl-2 target on cardiac ion channel activity including but not limited to the cardiac persistent sodium channel.

As would be known in the art, the methodologies above can also be used to determine the ability of a test compound to modulate the activity of a polypeptide that regulates bcl-2.

c. Cell-Free Assays

The present disclosure also includes cell-free assays. Such assays involve contacting a form of bcl-2 a bcl-2 target with a test compound and determining the ability of the test compound to bind to bcl-2 or a bcl-2 target or to inhibit bcl-2 or a bcl-2 target. Binding of the test compound to bcl-2 or such bcl-2 targets can be determined either directly or indirectly as described above. Regulation of bcl-2 activity or of a bcl-2 target may be determined as above. Such assays can employ a variety of variants of bcl-2 and bcl-2 targets, such as a full-length polypeptide, a biologically active fragment, or a fusion protein which includes all or a portion of the desired polypeptide.

In one embodiment, the assay includes contacting a cell free system containing bcl-2 or a bcl-2 target with a known compound to form an assay mixture, contacting the assay mixture with a test compound, and determining the ability of the test compound to interact with bcl-2 or a bcl-2 target, wherein determining the ability of the test compound to interact with bcl-2 or a bcl-2 target is determined by determining the ability of the test compound to preferentially bind to bcl-2 or a bcl-2 target as compared to the known compound.

The cell-free assays of the present disclosure are amenable to use of either a membrane-bound form of bcl-2 or a bcl-2 target or a soluble fragment thereof. In the case of cell-free assays comprising the membrane-bound form of the polypeptide, it may be desirable to utilize a solubilizing agent such that the membrane-bound form of the polypeptide is maintained in solution. Examples of such solubilizing agents include but are not limited to non-ionic detergents such as n-octylglucoside, n-dodecylglucoside, n-dodecyhnaltoside, octanoyl-N-methylglucamide, decanoyl-N-methylglucamide, Triton X-100, Triton X-114, Thesit, Isotridecypoly (ethylene glycol ether) n, 3-[(3-cholamidopropyl) dimethylamminio]-1-propane sulfonate (CHAPS), 3-[(3-cholamidopropyl) dimethylamminio]-2-hydroxy-1-propane sulfonate (CHAPSO), or N-dodecyl-N,N-dimethyl-3-ammonio-1-propane sulfonate. Therefore, the bcl-2 or a bcl-2 target may be expressed in a liposome, micelle or similar lipid containing structure

In various embodiments of the above assay methods, it may be desirable to immobilize bcl-2 or a bcl-2 target to facilitate separation of complexed from uncomplexed forms of one or both of the polypeptides, as well as to accommodate automation of the assay. Binding of a test compound to bcl-2 or a bcl-2 target or measuring the interaction of bcl-2 with a bcl-2 target in the presence and absence of a test compound, can be accomplished in any vessel suitable for containing the reactants.

Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase (GST) fusion proteins or glutathione-S-transferase fusion proteins can be adsorbed onto glutathione sepharose beads or glutathione derivatized microtitre plates, which are then combined with the test compound and the mixture incubated under conditions conducive to complex formation (for example at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of binding or activity of bcl-2 or a bcl-2 target can be determined using standard techniques.

d. Gene Expression

The screening assay can also involve monitoring the expression of bcl-2 or a bcl-2 target. For example, regulators of expression of bcl-2 or a bcl-2 target can be identified in a method in which a cell is contacted with a test compound and the expression of bcl-2 or a bcl-2 target or mRNA encoding the foregoing in the cell is determined. The level of expression of polypeptide or mRNA in the presence of the test compound is compared to the level of expression of in the absence of the test compound. The test compound can then be identified as a regulator of expression of bcl-2 or a bcl-2 target. For example, when expression of polypeptide or mRNA protein is decreased to a greater degree in the presence of the test compound than in its absence, the test compound is identified as an inhibitor of polypeptide or mRNA expression. The level of polypeptide or mRNA expression in the cells can be determined by methods described below.

The level of mRNA or polypeptide expression in the cells can be determined by methods well known in the art for detecting mRNA or polypeptide. Either qualitative or quantitative methods can be used. The presence of bcl-2 or a bcl-2 target polypeptide or nucleic acid can be determined, for example, using a variety of techniques known in the art, including immunochemical methods such as radio-immunoassay, Western blotting, Northern blots, Southern blots, microarray testing, PCR techniques, including but not limited to, real-time PCR and immuno-histochemistry. Alternatively, polypeptide synthesis can be determined in vivo, in a cell culture, or in an in vitro translation system by detecting incorporation of labeled amino acids into bcl-2 or a bcl-2 target.

Such screening can be carried out either in a cell-free assay system or in an intact cell as described herein. Such system may also be used to determine the stability of polypeptides or nucleic acid, including mRNA, encoding bcl-2 or a bcl-2 target.

e. Biophysical Measurement

Another embodiment of an assay to identify test compounds that bind to bcl-2 or a bcl-2 target is biophysical methods which measure the ability of such compounds to suppress BH3 domain peptide or other peptide binding to bcl-2 or bcl-2 targets. Such a general method is outlined in the work of Wang J L et al. “Structure-based discovery of an organic compound that binds bcl-2 protein and induces apoptosis of tumor cells” Proceedings of the National Academy of Science (U.S.A.) 97: 7124-7129 (2000), which reference is incorproated herein for such teaching. Here a competitive binding assay was described which used changes in fluorescence polarization to measure the interaction between bcl-2 or a bcl-2 target and test compounds. The authors define a general method wherein a peptide derived from the BH3 domain of BAK is labeled with a small fluorophore with appropriate relaxation properties to allow substantial changes in fluorescence polarization upon binding to bcl-2; as one example, 5-carboxyfluorescein. A recombinant glutathione-S-transferase (GST)-fused bcl-2 protein was produced from bacterial systems and purified. In this embodiment of a biophysical assay, GST-bcl-2 and the fluorescently-labeled peptide are mixed in control experiments and the decrease in peptide fluorescence polarization is measured. Decreases in polarization indicate peptide binding to the BH3 domain on the GST-bcl-2 polypeptide. These decreases are compared to values measured in solutions containing no GST-bcl-2 or a non-bcl-2 polypeptide as a negative control. Increasing concentrations of test compound are added to the solution and any change in peptide polarization is recorded. Test compounds that effectively increase or prevent peptide polarization will be considered candidate bcl-2 or bcl-2 target antagonists that may have anti-arrhythmic properties. Other embodiments of this general biophysical assay would include methods to measure changes in bcl-2 conformation in the presence of test compounds using other methods such as optical rotary dispersion or circular dichroism. Similar biophysical measurements can be made in cellular or molecular systems expressing a bcl-2 target.

f. Test Compounds

Suitable test compounds for use in the screening assays can be obtained from any suitable source, such as conventional compound libraries. The test compounds can also be obtained using any of the numerous approaches in combinatorial library methods known in the art, including: biological libraries, spatially addressable parallel solid phase or solution phase libraries, synthetic library methods requiring deconvolution, the “one-bead one-compound” library method and synthetic library methods using affinity chromatography selection. The biological library approach is limited to peptide libraries, while the other four approaches are applicable to peptide, non-peptide oligomer or small molecule libraries of compounds. Examples of methods for the synthesis of molecular libraries can be found in the art. Libraries of compounds may be presented in solution or on beads, bacteria, spores, plasmids or phage.

g. Modeling Compounds

Computer modeling and searching technologies permit identification of compounds, or the improvement of already identified compounds, that can inhibit bcl-2 or a bcl-2 target, either through expression, stability or activity. Having identified such a compound, the active sites or regions are identified. Such active sites might typically be ligand binding sites. The active site can be identified using methods known in the art including, for example, from the amino acid sequences of peptides, from the nucleotide sequences of nucleic acids, or from study of complexes of the relevant compound or composition with its natural ligand.

In the latter case, chemical or X-ray crystallographic methods can be used to find the active site by finding where on the factor the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intramolecular distances. Any other experimental method of structure determination can be used to obtain partial or complete geometric structures. The geometric structures may be measured with a complexed ligand, natural or artificial, which may increase the accuracy of the active site structure determined. If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, test compounds can be identified by searching databases containing compounds along with information on their molecular structure. Such a search seeks compounds having structures that match the determined active site structure and that interact with the groups defining the active site. Such a search can be manual, but is preferably computer assisted. Furthermore, such compounds may be synthesized or modified through peptidomimetic approaches based on the information obtained about the polypeptide as described above.

Alternatively, these methods can be used to identify improved compounds known in the art or identified in one of the screening assays above. These compounds found may be used to inhibit bcl-2 or a bcl-2 target.

### Compositions

Useful compositions of the present disclosure may comprise one or more compounds useful in the treatment and prevention methods of the present disclosure, such as, but not limited to, those compounds that modulate the expression, stability or activity of bcl-2 or a bcl-2 target; such compounds may be identified by a screening method of the present disclosure. Such compounds also include those compounds disclosed herein.

1. General Considerations

In one embodiment, such compounds are in the form of compositions, such as but not limited to, pharmaceutical compositions. The compositions disclosed may comprise one or more of such compounds, in combination with a pharmaceutically acceptable carrier. Examples of such carriers and methods of formulation may be found in Remington: The Science and Practice of Pharmacy (20th Ed., Lippincott, Williams & Wilkins, Daniel Limmer, editor). To form a pharmaceutically acceptable composition suitable for administration, such compositions will contain a therapeutically effective amount of a compound(s).

The pharmaceutical compositions of the disclosure may be used in the treatment and prevention methods of the present disclosure. Such compositions are administered to a subject in amounts sufficient to deliver a therapeutically effective amount of the compound(s) so as to be effective in the treatment and prevention methods disclosed herein. The therapeutically effective amount may vary according to a variety of factors such as, but not limited to, the subject's condition, weight, sex and age. Other factors include the mode and site of administration. The pharmaceutical compositions may be provided to the subject in any method known in the art. Exemplary routes of administration include, but are not limited to, subcutaneous, intravenous, topical, epicutaneous, oral, intraosseous, intramuscular, intranasal and pulmonary. The compositions of the present disclosure may be administered only one time to the subject or more than one time to the subject. Furthermore, when the compositions are administered to the subject more than once, a variety of regimens may be used, such as, but not limited to, one per day, once per week, once per month or once per year. The compositions may also be administered to the subject more than one time per day. The therapeutically effective amount of the molecule and appropriate dosing regimens may be identified by routine testing in order to obtain optimal activity, while minimizing any potential side effects. In addition, co-administration or sequential administration of other agents may be desirable.

The compositions of the present disclosure may be administered systemically, such as by intravenous administration, or locally such as by subcutaneous injection or by application of a paste or cream.

The compositions of the present disclosure may further comprise agents which improve the solubility, half-life, absorption, etc. of the compound(s). Furthermore, the compositions of the present disclosure may further comprise agents that attenuate undesirable side effects and/or or decrease the toxicity of the compounds(s). Examples of such agents are described in a variety of texts, such a, but not limited to, Remington: The Science and Practice of Pharmacy (20th Ed., Lippincott, Williams & Wilkins, Daniel Limmer, editor).

The compositions of the present disclosure can be administered in a wide variety of dosage forms for administration. For example, the compositions can be administered in forms, such as, but not limited to, tablets, capsules, sachets, lozenges, troches, pills, powders, granules, tinctures, solutions, suspensions, elixirs, syrups, ointments, creams, pastes, emulsions, or solutions for intravenous administration or injection. Other dosage forms include administration transdermally, via patch mechanism or ointment. Further dosage forms include formulations suitable for delivery by nebulizers or metered dose inhalers. Any of the foregoing may be modified to provide for timed release and/or sustained release formulations.

In the present disclosure, the pharmaceutical compositions may further comprise a pharmaceutically acceptable carrier. Such carriers include, but are not limited to, vehicles, adjuvants, surfactants, suspending agents, emulsifying agents, inert fillers, diluents, excipients, wetting agents, binders, lubricants, buffering agents, disintegrating agents and carriers, as well as accessory agents, such as, but not limited to, coloring agents and flavoring agents (collectively referred to herein as a carrier). Typically, the pharmaceutically acceptable carrier is chemically inert to the active compounds and has no detrimental side effects or toxicity under the conditions of use. The pharmaceutically acceptable carriers can include polymers and polymer matrices. The nature of the pharmaceutically acceptable carrier may differ depending on the particular dosage form employed and other characteristics of the composition.

For instance, for oral administration in solid form, such as but not limited to, tablets, capsules, sachets, lozenges, troches, pills, powders, or granules, the compound(s) may be combined with an oral, non-toxic pharmaceutically acceptable inert carrier, such as, but not limited to, inert fillers, suitable binders, lubricants, disintegrating agents and accessory agents. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, and the like. Disintegrators include, without limitation, starch, methyl cellulose, agar, bentonite, xanthum gum and the like. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid as well as the other carriers described herein. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

For oral liquid forms, such as but not limited to, tinctures, solutions, suspensions, elixirs, syrups, the nucleic acid molecules of the present disclosure can be dissolved in diluents, such as water, saline, or alcohols. Furthermore, the oral liquid forms may comprise suitably flavored suspending or dispersing agents such as the synthetic and natural gums, for example, tragacanth, acacia, methylcellulose and the like. Moreover, when desired or necessary, suitable coloring agents or other accessory agents can also be incorporated into the mixture. Other dispersing agents that may be employed include glycerin and the like.

Formulations suitable for parenteral administration include aqueous and non-aqueous, isotonic sterile injection solutions, which can contain anti-oxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The compound(s) may be administered in a physiologically acceptable diluent, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as, but not limited to, a soap, an oil or a detergent, suspending agent, such as, but not limited to, pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include polyethylene sorbitan fatty acid esters, such as sorbitan monooleate and the high molecular weight adducts of ethylene oxide with a hydrophobic base, formed by the condensation of propylene oxide with propylene glycol, oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkylbeta-aminopropionates, and 2-alkylimidazoline quaternary ammonium salts, and (e) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17.

Topical dosage forms, such as, but not limited to, ointments, creams, pastes, emulsions, containing the molecule of the present disclosure, can be admixed with a variety of carrier materials well known in the art, such as, e.g., alcohols, aloe vera gel, allantoin, glycerine, vitamin A and E oils, mineral oil, PPG2 myristyl propionate, and the like, to form alcoholic solutions, topical cleansers, cleansing creams, skin gels, skin lotions, and shampoos in cream or gel formulations. Inclusion of a skin exfoliant or dermal abrasive preparation may also be used. Such topical preparations may be applied to a patch, bandage or dressing for transdermal delivery or may be applied to a bandage or dressing for delivery directly to the site of a wound or cutaneous injury.

The compound(s) of the present disclosure can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. Such liposomes may also contain monoclonal antibodies to direct delivery of the liposome to a particular cell type or group of cell types.

The compound(s) of the present disclosure may also be coupled with soluble polymers as targetable drug carriers. Such polymers can include, but are not limited to, polyvinyl-pyrrolidone, pyran copolymer, polyhydroxypropylmethacryl-amidephenol, polyhydroxyethylaspartamidephenol, or polyethyl-eneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

2. Small Molecule bcl-2 and bcl-2-Target Inhibitors

The present disclosure teaches several small molecules unexpectedly discovered to be capable of treating or preventing SMA and/or arrhythmia in a subject. In one embodiment, small molecule compounds may inhibit the activity, stability and/or expression of bcl-2 or a bcl-2 target. In another embodiment, small molecule compounds may modulate calcium homeostasis in a cell (such as a myocyte); for example, such small molecule compounds may modulate the leak of calcium from myocyte stores and/or the entry of calcium into the cell via the SOC. The small molecules include polyphenols, bcl-2 ligands, polyphenolic aldehydes, catechins, antioxidants, flavonols, flavonoids, phytochemicals, antibiotics, antibiotic derivatives, phytoalexins, stilbenoids, and synthetics. Other small molecules include small peptides or peptidomimetics that act as bcl-2 or bcl-2 target inhibitors. Particular examples include HA14-1, gossypol, quercetin, EGCG, EGC, 2-methoxy antimycin A, resveratrol, and SKF-96365. Such exemplars and others can be identified using the methods described herein.

3. Nucleic Acid bcl-2 and bcl-2-Target Inhibitors

In one embodiment, the compounds of the present disclosure that inhibit the activity, expression or stability of bcl-2 or a bcl-2 target are functional nucleic acids. Functional nucleic acids are nucleic acid molecules that carry out a specific function in a cell, such as binding a target molecule or catalyzing a specific reaction.

Functional nucleic acids include but are not limited to antisense molecules, aptamers, ribozymes, triplex forming molecules, small interfering RNA (siRNA), RNA interference (RNAi), and external guide sequences (EGS). In one embodiment, a siRNA could be used to reduce or eliminate expression of bcl-2 or a bcl-2 target.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively, the antisense molecule is designed to interrupt a processing function that normally would take place on the target nucleic acid molecule, such as transcription or replication. Antisense molecules can be designed based on the sequence of the target nucleic acid molecule. Numerous methods for optimization of antisense efficiency by finding the most accessible regions of the target nucleic acid molecule exist. Exemplary methods include, but are not limited to, in vitro selection experiments and DNA modification studies using DMS and DEPC.

Aptamers are molecules that interact with a target nucleic acid molecule, preferably in a specific way. Typically aptamers are small nucleic acids ranging from 15-50 bases in length that fold into defined secondary and tertiary structures, such as stem-loops or G-quartets. Representative examples of how to make and use aptamers to bind a variety of different target molecules can be found in, for example, U.S. Pat. Nos. 5,476,766 and 6,051,698, which are hereby incorporated by reference for such teaching. The secondary structure inhibits expression of the polypeptide encoded by the gene or inhibits a processing function as discussed above.

Ribozymes are nucleic acid molecules that are capable of catalyzing a chemical reaction, either intramolecularly or intermolecularly. There are a number of different types of ribozymes that catalyze nuclease or nucleic acid polymerase type reactions which are based on ribozymes found in natural systems, such as, but not limited to, hammerhead ribozymes, hairpin ribozymes and Tetrahymena ribozymes. There are also a number of ribozymes that are not found in natural systems, but which have been engineered to catalyze specific reactions de novo (including, but not limited to, those described in U.S. Pat. Nos. 5,807,718, and 5,910,408, which are hereby incorporated by reference for such teaching). Ribozymes may cleave RNA or DNA substrates. Representative examples of how to make and use ribozymes to catalyze a variety of different reactions can be found in U.S. Pat. Nos. 5,837,855; 5,877,022; 5,972,704; 5,989,906; and 6,017,756, which are hereby incorporated by reference for such teaching.

Triplex forming functional nucleic acid molecules are nucleic acid molecules that can interact with either double-stranded or single-stranded nucleic acid. When triplex forming nucleic acids interact with a target region, a structure called a triplex is formed, in which the three strands of DNA form a complex dependant on both Watson-Crick and Hoogsteen base-pairing. Triplex molecules can bind target regions of DNA with high affinity and specificity. Representative examples of how to make and use triplex forming molecules to bind a variety of different target molecules can be found in U.S. Pat. Nos. 5,650,316; 5,683,874; 5,693,773; 5,834,185; 5,869,246; 5,874,566; and 5,962,426, which are hereby incorporated by reference for such teaching.

EGSs are molecules that bind a target nucleic acid molecule forming a complex, which is recognized by RNase P. RNase P then cleaves the target nucleic acid molecule. EGSs can be designed to specifically target a RNA molecule of choice. Representative examples of how to make and use EGS molecules to facilitate cleavage of a variety of different target molecules be found in U.S. Pat. Nos. 5,168,053; 5,624,824; 5,683,873; 5,728,521; 5,869,248; and 5,877,162, which are hereby incorporated by reference for such teaching.

Gene expression can also be effectively silenced in a highly specific manner through RNAi. siRNA is a double-stranded RNA that can induce sequence-specific post-transcriptional gene silencing, thereby decreasing or even inhibiting gene expression from a target nucleic acid. In one example, an siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme dicer. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. siRNA can also be synthesized in vitro using kits such as Ambion's SILENCER® siRNA Construction Kit (Ambion, Austin, Tex.).

4. Antibody bcl-2 and bcl-2-Target Inhibitors

Polypeptides that inhibit the activity, expression or stability of bcl-2 or a bcl-2 include antibodies with antagonistic or inhibitory properties. In addition to intact immunoglobulin molecules, fragments, chimeras, or polymers of immunoglobulin molecules are also useful in the methods taught herein. The antibodies can be tested for their desired activity using in vitro assays, or by analogous methods, after which their in vivo therapeutic or prophylactic activities are tested according to known clinical testing methods.

The twin antibody is used herein in a broad sense and includes polyclonal, monoclonal, and single-chain phage display antibodies. Monoclonal antibodies can be made using any known procedure. For example, disclosed monoclonal antibodies can be prepared using hybridoma methods, such as those described by Kohler and Milstein, Nature 256:495 (1975) which is hereby incorporated by reference for such teaching. In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro. The monoclonal antibodies may also be made by recombinant DNA methods, such as those described in U.S. Pat. No. 4,816,567, which is hereby incorporated by reference for such teaching. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, as described in U.S. Pat. No. 5,804,440 and U.S. Pat. No. 6,096,441, which are hereby incorporated by reference for such teaching.

Antibody fragments include Fv, Fab, Fab′ or other antigen binding portions of an antibody. Digestion of antibodies to produce fragments thereof can be accomplished using routine techniques known in the art. For instance, digestion can be performed using papain. Examples of papain digestion are described in WO 94/29348 published and U.S. Pat. No. 4,342,566, which are hereby incorporated by reference for such teaching. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross linking antigen.

The antibodies or antibody fragments may also include insertions, deletions, substitutions, or other selected modifications of particular regions or specific amino acids residues. These modifications can provide additional or improved function. For example, the removal or addition of amino acids capable of disulfide bonding may increase the bio-longevity of the antibody. In any case, the modified antibody or antibody fragment retains a desired bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Current. Opinions Biotechnology 3:348-354, (1992), which is hereby incorporated by reference for such teaching).

As used herein, the term antibody or antibodies can also refer to a human antibody and/or a humanized antibody. Examples of techniques for human monoclonal antibody production include those described by Cole et al. (Monoclonal Antibodies and Cancer Therapy, Alan R. Liss, p. 77, 1985) and by Boerner et al. (J. Immunology, 147: 86-95 (1991), which are hereby incorporated by reference for such teaching). Human antibodies (and fragments thereof) can also be produced using phage display libraries (Hoogenboom et al., J. Molecular Biology 227: 381 (1991); Marks et al., J. Molecular Biology, 222: 581 (1991), which are hereby incorporated by reference for such teaching). The disclosed human antibodies can also be obtained from transgenic animals. For example, transgenic, mutant mice that are capable of producing a full repertoire of human antibodies, in response to immunization, have been described (see, e.g., Jakobovits et al., Proceedings National Academy of Science (USA) 90: 2551-2555 (1993); Jakobovits et al., Nature 362: 255-258 (1993); Bruggermann et al., Year in Immunology, 7: 33 (1993), which are hereby incorporated by reference for such teaching).

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain that contains a portion of an antigen binding site from a non-human (donor) antibody integrated into the framework of a human (recipient) antibody. Fragments of humanized antibodies are also useful in the methods taught herein. Methods for humanizing non human antibodies are well known in the art. For example, humanized antibodies can be generated according to the methods of Winter and co workers (Jones et al., Nature 321: 522-525 (1986), Riechmann et al., Nature 332: 323-327 (1988), Verhoeyen et al., Science 239: 1534-1536 (1988), which are hereby incorporated by reference for such teaching), by substituting rodent CDRs or CDR sequences for the corresponding sequences of a human antibody. Methods that can be used to produce humanized antibodies are also described in U.S. Pat. Nos. 4,816,567, 5,565,332, 5,721,367, 5,837,243, 5,939,598, 6,130,364, and 6,180,377, which are hereby incorporated by reference for such teaching.

### C. METHODS OF TREATMENT AND PREVENTION OF ARRHYTHMIA

Methods of treatment and prevention of arrhythmia and SMA in a subject are provided by the present disclosure and comprise administering to the subject a compound disclosed herein or a compound identified by the methods disclosed herein. Such compounds may inhibit the activity, expression or stability of bcl-2 or a bcl-2 target or alter calcium homeostasis in a cell (such as a myocyte).

Many of the compounds described above have been subject to medical studies. Those skilled in the art can discern from these studies appropriate therapeutic doses to the extent that no undue experimentation would be necessary to practice these embodiments of the methods. Those skilled in the art could turn to scholarly reviews of the literature, individual studies, and data from clinical trials for specific references.

In light of the published data available to those skilled in the art pertaining to the in vitro, animal, and human studies of the inhibitors, and further in light of this disclosure's description of the effects of the inhibitor on isolated cardiac muscle, it is well within the capabilities of those skilled in the art to practice embodiments of the method.

Embodiments of the method include a method of treating or preventing arrhythmia, the method comprising administering to a subject a therapeutically effective amount of a compound that inhibits that inhibits the activity or expression of bcl-2 or a bcl-2 target.

In an alternate embodiment, the present disclosure provides a method of treating or preventing arrhythmia, the method comprising administering to the subject a therapeutically effective amount of a compound that inhibits an ion channel active during an arrhythmic event, wherein the ion channel is selected from the group consisting of a sodium channel and a calcium channel. In one embodiment, the ion channel is a voltage-dependent calcium channel, such as, but not limited to, a ryanodine channel. In another embodiment, the ion channel is a voltage-independent calcium channel, such as, but not limited to, a calcium channel regulated by an IP3R or a SOC. In still another embodiment, the ion channel is a sodium channel, such as, but not limited to the persistent sodium ion channel or a sodium channel activated by an increase in intracellular calcium.

The compound may inhibit the activity, expression and/or stability of bcl-2 or a bcl-2 target. In certain embodiments, administering occurs after an onset of arrhythmia. In further embodiments, the compound may be EGCG, gossypol, 2-methoxy antimycin A, HA14-1, ECG, quercetin, resveratrol, ABT-737, SKF-96365, their derivatives or a combination thereof. In an alternative embodiment of the method, the compound comprises a functional nucleic acid, for example an antisense molecule, an aptamer, a ribozyme, a triplex forming molecule, a short interfering RNA, an external guide sequence, or a combination thereof. In another alternative embodiment of the method, the compound comprises an inhibitory polypeptide, for example a peptide targeted to a bcl-2 or a bcl-2 target, an immunoglobulin molecule, a fragment of an immunoglobulin molecule, a chimeric immunoglobulin, a fragment of a chimeric immunoglobulin, a polymer of immunoglobulin molecules, or a combination thereof.

In further embodiments, the method further comprises identifying a subject in need of treatment or prevention of arrhythmia or SMA. In further embodiments, the subject experiences a frequency of arrhythmia events, and the method further comprises reducing the frequency of arrhythmia events. In further embodiments, the subject experiences an arrhythmia event prior to, during or after the administering step.

In some embodiments of the method, the method further comprises reversing an arrhythmia event.

In another embodiment, the critical sites noted above for bcl-2 or a bcl-2 target or a compounds, such as but not limited to a peptide, that binds to these sites are used to construct a molecule with inhibitory structural and electrostatic properties using the peptidomimetic approach (for example Wu and Gellman, Peptidomimetics, Accounts of Chemical Research 41, 1231-1232 (2008) which is incorporated by reference for such teaching).

In embodiments of the method comprising identifying a subject in need of treatment or prevention, the identification of the subject may occur prior to the administration of the compound. However, under conditions in which the administration of the compound has diagnostic value, the identification of the subject may occur after administration. Compounds of the present disclosure may be administered alone or in combination with other pharmaceutical agents known in the art to be of value in treating, preventing or diagnosing arrhythmia or SMA. When a combination is administered, the individual compounds may be administered simultaneously at a given dosage time, or they may be interspersed at varying, intermittent, or alternating dosage times. Administration may occur at any time relative to an onset of arrhythmia. Administration may occur in a patient who has never experienced arrhythmia or SMA, for preventive purposes. Administration may occur at a time after a patient has experienced a discrete occurrence of arrhythmia or SMA, generally (but not necessarily) to prevent a recurrence. Administration may occur during an arrhythmia event or SMA, to reverse an arrhythmia or SMA and restore normal rhythmic function to the heart.

### D. METHODS OF DIAGNOSIS

The present disclosure also provides methods for diagnosis or determining if a subject is suffering from or at risk for a arrhythmia or SMA and disease states and conditions associated with or characterized by increased bcl-2 activity increased bcl-2 target activity, such as arrhythmia or SMA.

In a general embodiment, the method comprises obtaining a biological sample from a first subject whose risk for arrhythmia or SMA is to be determined and a control subject who has been determined not to be at risk for arrhythmia or SMA, measuring a level of expression or activity of bcl-2 or a bcl-2 target in the first subject and the control subject, comparing the level of expression or activity in the first subject and the control subject, wherein an increase in the level of expression or activity indicates an increased risk for arrhythmia or SMA.

In one embodiment, the methods for diagnosis involve determining the status of a subject with respect to the activity and/or expression of bcl-2 or a bcl-2 target. As used herein a biological sample which is subjected to testing is a sample derived from a subject and includes, but is not limited to, any biological fluid, including a bodily fluid. Examples of bodily fluids include, but are not limited to whole blood, serum, saliva, tissue infiltrate, pleural effusions, lung lavage fluid, bronchoalveolar lavage fluid, and the like. The biological fluid may be a cell culture medium or supernatant of cultured cells. For example, the sample can be a blood sample or a serum sample. Additional examples of biological samples include heart tissue samples or biopsies.

Those subjects in which the activity or expression of bcl-2 or a bcl-2 target is increased as compared to a control subject or increased above a control value set in the art are determined to be suffering from or at risk.

In one embodiment, the difference is at least 1.25-fold, 1.5-fold, 2-fold, 5-fold or higher. A control means a sample obtained from a subject that does not exhibit a symptom or characteristic of the disease state or condition to be determined. The control value may be determined empirically from a subject or group of subjects or may be an average value from a selected population.

Assay techniques that can be used to determine levels of expression or activity in a sample are known. Such assay methods include, but are not limited to, radio-immunoassays, reverse transcriptase PCR (RT-PCR) assays, immuno-histochemistry assays, in situ hybridization assays, competitive-binding assays, Western Blot analyses, ELISA assays and proteomic approaches, two-dimensional gel electrophoresis (2D electrophoresis) and non-gel based approaches such as mass spectrometry or protein interaction profiling. Assays also include, but are not limited to, competitive and non-competitive assay systems using techniques such as radio-immunoassays, enzyme immunoassays (EIA), enzyme linked immunosorbent assay (ELISA), sandwich immunoassays, precipitation reactions, gel diffusion reactions, immunodiffusion assays, agglutination assays, complement-fixation assays, immuno-radiometric assays, fluorescent immunoassays, protein A immunoassays, and immuno-electrophoresis assays. For examples of immunoassay methods, see U.S. Pat. No. 4,845,026 and U.S. Pat. No. 5,006,459.

In an ELISA assay, an antibody is prepared, if not readily available from a commercial source, specific to an antigen, such as, for example, bcl-2 or a bcl-2 target. In addition, a reporter antibody generally is prepared. The reporter antibody is attached to a detectable reagent such as a radioactive, fluorescent or enzymatic reagent, for example horseradish peroxidase enzyme or alkaline phosphatase. In one embodiment of the ELISA, to carry out the ELISA, antibody specific to antigen is incubated on a solid support that binds the antibody. Any free protein or molecule binding sites on the dish are then covered by incubating with a non-specific protein. Next, the sample to be analyzed is incubated with the solid support, during which time the antigen binds to the specific antibody. Unbound sample is washed out with buffer. A reporter antibody specifically directed to the antigen and linked to a detectable reagent is introduced resulting in binding of the reporter antibody to any antibody bound to the antigen. Unattached reporter antibody is then washed out. Reagents for detecting the presence of the reporter antibody are then added. The detectable reagent is then determined in order to determine the amount of antigen present. In an alternate embodiment, the antigen is incubated with the solid support, followed by incubation with one or more antibodies, wherein at least one of the antibodies comprises a detectable reagent. Quantitative results may be obtained by reference to a standard curve.

Optionally, a genetic sample from the biological sample can be obtained. The genetic sample comprises a nucleic acid, preferably RNA and/or DNA. For example, in determining the expression of genes, mRNA can be obtained from the biological sample, and the mRNA may be reverse transcribed into cDNA for further analysis. Alternatively, the mRNA itself is used in determining the expression of genes. A genetic sample may be obtained from the biological sample using any techniques known in the art (Ausubel et al. Current Protocols in Molecular Biology John Wiley & Sons, Inc., New York (1999); Molecular Cloning: A Laboratory Manual, 2nd Ed., ed. by Sambrook, Fritsch, and Maniatis Cold Spring Harbor Laboratory Press (1989); Nucleic Acid Hybridization B. D. Hames & S. J. Higgins eds. (1984) each of the foregoing being incorporated herein by reference). The nucleic acid may be purified from whole cells using DNA or RNA purification techniques. The genetic sample may also be amplified using PCR or in vivo techniques requiring sub-cloning. The genetic sample can be obtained by isolating mRNA from the cells of the biological sample and reverse transcribing the RNA into DNA in order to create cDNA (Khan et al. Biochem. Biophys. Acta 1423:17-28 (1999).

Once a genetic sample has been obtained, it can be analyzed. The analysis may be performed using any techniques known in the art including, but not limited to, sequencing, PCR, RT-PCR, quantitative PCR, restriction fragment length polymorphism, hybridization techniques, Northern blot, microarray technology, and similar techniques. In determining the expression level of a gene or genes in a genetic sample, the level of expression may be normalized by comparison to the expression of another gene such as a well known, well characterized gene or a housekeeping gene (for example, actin). For example, reverse-transcriptase PCR (RT-PCR) can be used to detect the presence of a specific mRNA population in a complex mixture of thousands of other mRNA species.

Hybridization to clones or oligonucleotides arrayed on a solid support (i.e., gridding) can be used to both detect the expression of and measure the level of expression of that gene. In this approach, a cDNA encoding an antigen is fixed to a substrate. The substrate may be of any suitable type including but not limited to glass, nitrocellulose, nylon or plastic. At least a portion of the DNA encoding the antigen is attached to the substrate and then incubated with the analyte, which may be RNA or a complementary DNA (cDNA) copy of the RNA, isolated from the sample of interest. Hybridization between the substrate bound DNA and the analyte can be detected and measured by several means including but not limited to radioactive labeling or fluorescence labeling of the analyte or a secondary molecule designed to detect the hybrid. Quantitation of the level of gene expression can be done by comparison of the intensity of the signal from the analyte compared with that determined from known standards. The standards can be obtained by in vitro transcription of the target gene, measuring the yield, and then using that material to generate a standard curve.

### E. KITS

The present disclosure provides kits for carrying out any method of the present disclosure, which can contain any of the compounds and/or compositions disclosed herein or otherwise useful for practicing a method of the disclosure.

For example, the disclosure provides a kit for the identification of anti-arrhythmia agents, the kit comprising instructions for carrying out an assay or method of the present disclosure and at least one of the following components: a myocyte (either isolated or as a part of a cardiac muscle), a physiologically suitable buffer, an arrhythmic agent, a calcium loading compound, a candidate anti-arrhythmic agent, an cell comprising bcl-2 or a bcl-2 target, an isolated bcl-2 or bcl-2 target polypeptide, a pace-making device and an instrument suitable for measuring mechanical or electrical activity of the myocyte.

The myocytes or cardiac muscles may be isolated from genetically normal animals or from animals genetically modified to have altered activity, expression or stability of bcl-2 or a bcl-2 target. In further embodiments the isolated cardiac muscle comprises a left atrial appendage, a ventricular muscle strip, a right ventricular muscle strip, a papillary muscle, an intact heart or a myocyte. In further embodiments, the buffer comprises KH perfusate or related solutions including but not limited to whole blood or blood components. In further embodiments, the pace-making device comprises a field or point stimulator such as a Grass S44 or S88 Stimulator. In further embodiments, the mechanical force recorder comprises a force transducer, for example a Grass FT-3 transducer, coupled to an appropriate recording device, for example, a Grass Model 7D Polygraph. Electrical activity can be measured as described above In further embodiments, the kit further comprises a known anti-arrhythmia agent, and a substance that is known to have no effect on arrhythmia.

The disclosure provides for further embodiments of the kit, wherein the kit further comprises an agent capable of increasing cytoplasmic calcium. In some embodiments of the kit, the agent capable of increasing cytoplasmic calcium may be selected from the group consisting of but not limited to: activators of β-adrenergic signaling, activators of cAMP signaling, slow calcium channel activators, ouabain, agents that prolong the action potential duration, alter a cardiac sodium channel and a combination thereof. In further embodiments, the agent capable of increasing cytoplasmic calcium comprises isoproterenol, forskolin, BayK 8644, FPL-64176, ATX-II, (clofilium+a Gαq agonist) or ouabain. In other embodiments these agents may be a genetic modification which affects cardiac calcium homeostasis so as to activate automaticity, alter sodium channel properties or affect bcl-2 activity or a bcl-2 target activity so as to activate automaticity.

The foregoing description and the examples below illustrate and describe the methods and other teachings of the present disclosure. Additionally, the disclosure shows and describes only certain embodiments of the methods and other teachings disclosed, but, as mentioned above, it is to be understood that the teachings of the present disclosure are capable of use in various other combinations, modifications, and environments and is capable of changes or modifications within the scope of the teachings as expressed herein, commensurate with the skill and/or knowledge of a person having ordinary skill in the relevant art. The embodiments described hereinabove are further intended to explain certain best modes known of practicing the methods and other teachings of the present disclosure and to enable others skilled in the art to utilize the teachings of the present disclosure in such, or other, embodiments and with the various modifications required by the particular applications or uses. Accordingly, the methods, compositions, and other teachings of the present disclosure are not intended to limit the exact embodiments and examples disclosed herein.

### F. EXAMPLES

### Example 1

### Development of a Model System for Arrhythmia

The present disclosure contemplates that unregulated myocyte calcium release, calcium leak from ryanodine-insensitive, voltage-independent calcium stores and/or voltage-independent calcium entry may be responsible for the occurrence of SMA and arrhythmia. Since dysregulated calcium entry or myocyte calcium leak may underlie these electromechanical irregularities, experiments were conducted to determine whether 2-APB, a calcium leak/entry-inducer, affects mechanical function in isolated, superfused rat left atria (in the context of this example, the term “left atria” encompasses the use of the left atrial appendage). Exposing left atria paced at 3 Hz to >10 M 2-APB produced sporadic mechanical events that occurred in the absence of pacing stimulus. Prolonging the interval between atrial stimulation in the presence of 2-APB revealed the occurrence of SMA. SMA depends on atrial sodium and chloride gradients as decreasing superfusate concentration of either ion suppressed SMA. Increasing superfusate potassium to produce an E_{K} of ˜−74 mV reversed SMA, revealing possible membrane potential sensitivity. Mechanical function decreased with time in left atria treated with 2-APB and low sodium or the anion transport inhibitor 4,4′-diisothiocyanatostilbene-2,2′-disulfonic acid (DIDS) compared with atria exposed to low sodium or DIDS alone, suggesting 2-APB may decrease left atrial SR activator calcium. Thus, 2-APB produces instability in regular left atrial mechanical activity that may require forward-mode sodium-calcium exchange and chloride channel activities. These data identify a novel model for studying atrial contractile abnormalities, such as but not limited to, arrhythmia and SMA.

Calcium leakage can activate depolarizing currents that produce ectopic mechanical events in isolated myocytes. However few models are available to investigate whether calcium leakage underlies ectopic mechanical events in intact heart muscle preparations. Experimental results described below demonstrate that 2-APB produces instability in the regular electromechanical activity of isolated rat left atria (i.e., SMEs and SMA) and depresses the maximal mechanical function of these isolated preparations, a measure of SR activator calcium. These results identify a new pharmacological model with potential in studying mechanical abnormalities in isolated left atria under controlled conditions.

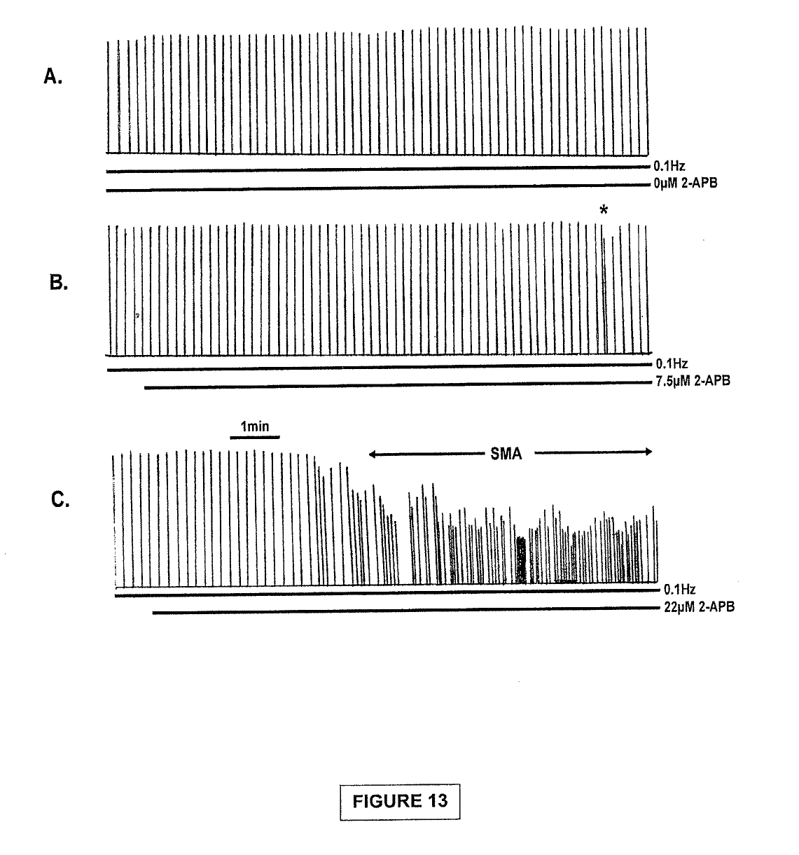

Instability of regular electromechanical function was observed to occur in left atria exposed to 2-APB (FIGS. 3 & 12) and became more pronounced as diastolic interval was lengthened. In the extreme, at pacing rates of 0.1 Hz and 2-APB concentrations of 15 μM numerous atrial mechanical events occurred in the absence of a pacing stimulus, SMA (FIG. 13). Under these conditions heart muscle produced normal looking action potentials that occurred in the absence of external stimulation and preceded spontaneous mechanical events (FIG. 3D; right panel). SMA appears to result from a specific interaction between 2-APB and left atria rather than from general atrial disruption, since diastolic tension did not increase in 2-APB-treated preparations (e.g., FIGS. 13 and 17), and removing 2-APB from the superfusate reversed its effects on atrial mechanical function.

Without wishing to be limited by any hypothetical model, one interpretation of these results is that SMA occurs as a result of depolarizations that arise in left atria exposed to moderate concentrations of 2-APB. Experimental evidence shows that increased forward-mode NCX (cardiac sodium/calcium exchanger) activity can cause fluctuations in myocyte membrane potential that trigger aberrant depolarizations. It has been suggested that calcium-activated chloride channels also contribute to these fluctuations, which elicit spontaneous depolarizations and ectopic mechanical events. Since NCX and calcium-activated chloride channels have different kinetic properties and appear to access different pools of myocyte calcium, some have hypothesized that both forward-mode NCX and calcium-activated chloride channels contribute to producing ectopic mechanical events.

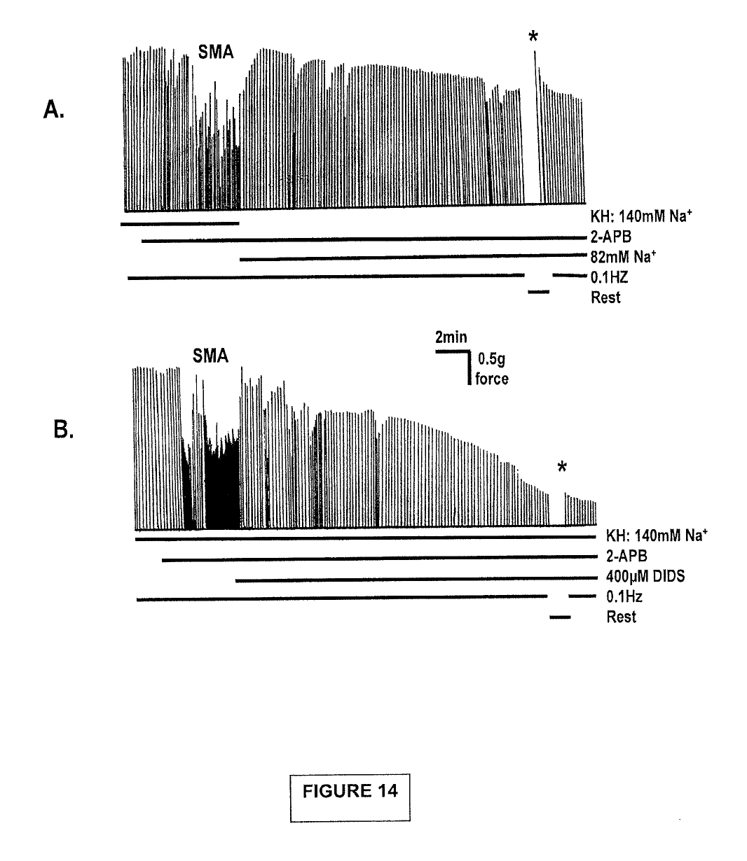

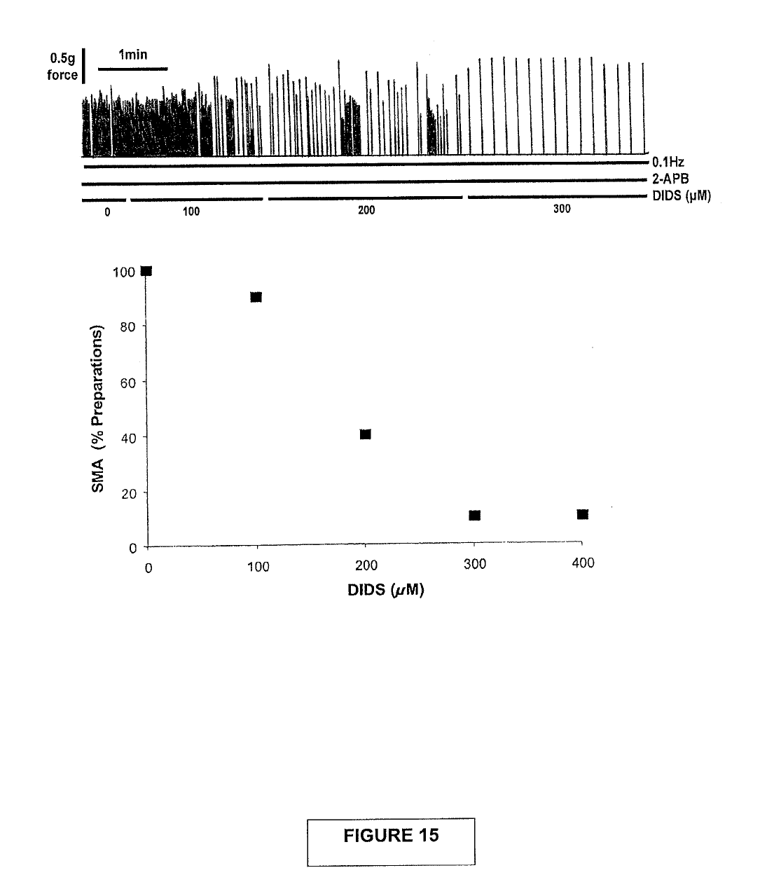

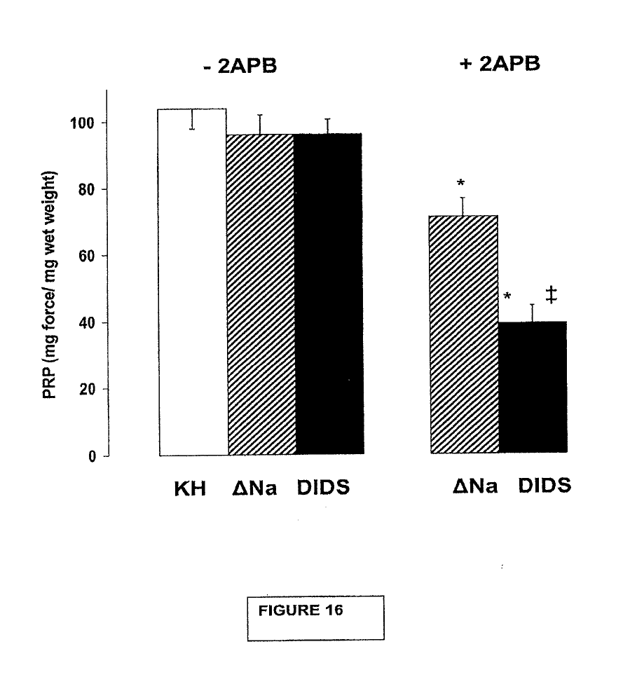

Six sets of results address this question. Decreasing either superfusate sodium or chloride prevented and reversed SMA (FIG. 14). DIDS also reversed SMA at concentrations shown previously to block anion transport (FIG. 15). These results suggest that both sodium and chloride gradients and anion transporters may be important in initiating and maintaining SMA. The fact that increasing superfusate potassium also reversed SMA (FIG. 11) suggests a membrane potential-sensitive step mediates, in part, SMA. These increases in superfusate potassium produced a calculated E_{K} of ˜−74 mV. Such a change, under conditions of normal superfusate sodium and calcium, may suppress forward-mode NCX activity activated by an increase in intracellular calcium. Taken together, the ion substitution and transport inhibitor studies suggest a role for both forward-mode NCX activity and chloride channel activity in initiating and maintaining left atrial SMA.

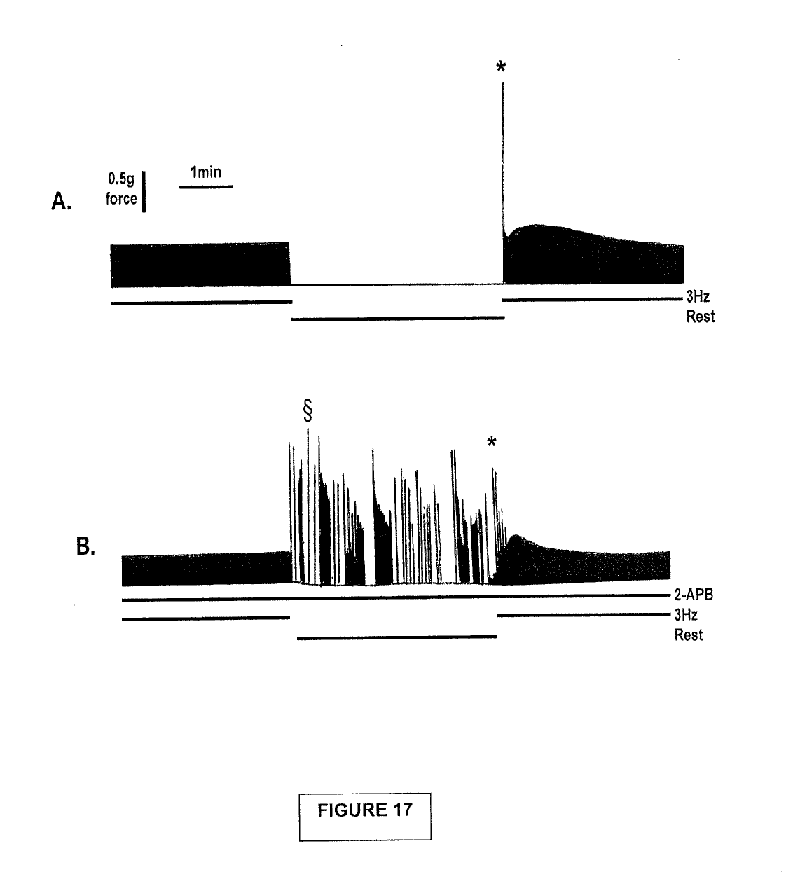

Lowering superfusate sodium or DIDS treatment acutely reversed SMA despite the continued presence of 2-APB (FIG. 14). Prolonged superfusion of 2-APB-treated left atria in the presence of low sodium or DIDS produced a decline in the maximum force of atrial contraction when compared with preparations treated with low sodium or DIDS alone. Others have reported that the first potentiated beat following prolonged rest, the maximum force of contraction, reflects the SR content of activator calcium. The present results suggest that 2-APB decreases the activator calcium available to generate mechanical force in isolated left atria. Importantly, atrial mechanical function was better preserved in the presence of 2-APB when low sodium was used to reverse SMA compared with when DIDS was used (FIGS. 14 and 16). The accelerated loss of maximum mechanical force in (2-APB+DIDS)-treated atria suggests that the activator calcium pool may decrease more in atria where forward-mode NCX activity should be unfettered. In contrast, lowering superfusate sodium should suppress forward-mode NCX activity, so this procedure to reverse SMA might slow the loss of atrial calcium via NCX and better preserve atrial mechanical performance. Under conditions of low superfusate sodium, sarcolemmal calcium ATPase may be responsible for calcium efflux from atrial myocytes.

Again without wishing to be limited by any hypothetical model, alternative explanations present themselves for the decreased maximum mechanical performance occurring in the presence of 2-APB. One is that 2-APB alters atrial myofilament calcium sensitivity either directly or by altering atrial metabolism to produce acidosis. The fact that atrial contraction and relaxation times were similar in left atria undergoing SMA or regular mechanical activity argues against this possibility as atrial relaxation times might change if altered myofilament calcium sensitivity occurred in 2-APB-treated preparations. Nonetheless, direct measure of myofilament calcium sensitivity is required to establish whether sensitivity is constant under the conditions described here. A second alternative explanation is that 2-APB alters atrial myocyte SR calcium release and accumulation. However, the similar contraction and relaxation times measured in the presence and absence of 2-APB again suggests that SR calcium release and accumulation are not greatly affected here. Third, 2-APB may alter the activity of atrial ion channels, including the slow calcium and sodium channels.

Some properties specific to atrial compared with ventricular myocytes may afford unique or enhanced triggers for instability in regular left atrial mechanical activity. It has been reported that enhanced IP3R calcium signaling leads to membrane depolarization and SR calcium release in isolated atrial myocytes and suggested that these abnormalities occur via NCX and/or the calcium-activated chloride channel activity present in atrial myocytes. Earlier reports also show high levels of type 2 IP3R occur in atrial compared with ventricular myocytes and that IP3R signaling can contribute to instability in atrial myocyte mechanical function. Finally, moderate concentrations of 2-APB alter the coupling process between IP3R and SOCs in non-excitable cells, causing leakage from store-operated calcium depots. In particular, prior art has shown that 2-APB opens the store-operated calcium channel at the concentrations which elicit SMA. This would produce a low-level steady increase in myocyte calcium in the region immediately below the cell plasma membrane. The latter event, if it occurs in isolated left atria, could contribute to results reported here.

The conclusions that can be drawn from the experiments described below are that moderate concentrations of 2-APB produce left atrial SMA, instability in the regular electrical and mechanical activity of these isolated muscles, which may require forward-mode NCX and chloride channel activities. These data identify a pharmacological approach that produces atrial ectopy that may contribute to arrhythmias.

a. Material and Methods

i. Preparation of Isolated Rat Left Atria and Measurement of Atrial Mechanical Function

Left atria were isolated from male Sprague-Dawley rats (325 to 400 g) under IP pentobarbital anesthesia (0.1 g/kg). Isolated left atria were superfused at 30° C. in Krebs-Henseleit (KH) buffer of the following mM composition: NaCl, 118; NaHCO3, 27; KCl, 4.8; MgSO4, 1.2; KH2PO4, 1.0; CaCl2, 1.8; and glucose, 11.1. Nearly isometric left atrial forces of contraction were measured as described.

ii. Four Protocols Analyzing the Effect of 2-APB on Atrial Mechanical Function

Isolated superfused left atria were paced at 3 Hz. 2-APB was added to atrial superfusates from 0.015 or 0.15M DMSO stock solutions to achieve concentrations of 0, 2, 7.5, 15, or 22 μM (n=5 to 10 atria per concentration). Changes in atrial mechanical force were recorded during a subsequent 15 min superfusion. DMSO alone did not affect atrial mechanical function.

Two groups of 5 to 6, 3 Hz paced atria were superfused in KH and exposed to 0 or 15 μM 2-APB for 10 min. The pacing stimulus was interrupted for 5 min and the number of spontaneous mechanical events (SMEs), defined as increases in mechanical force occurring in the absence of pacing, were measured as was the postrest potentiation response (PRP).

To determine whether diastolic interval affected the interaction between 2-APB and left atria, atrial pacing was decreased to 0.1 Hz, and 0, 7.5, or 22 μM 2-APB was added to the superfusate (n=6-8 per group). Superfusions continued for 15 min, and changes in atrial mechanical function were observed. Under these conditions, numerous SMEs occurred, and this phenomenon was designated spontaneous mechanical activity (SMA).

To establish whether SMA was reversible, atria (n=7) were paced at 0.1 Hz, exposed to 15 μM 2-APB for 10 min, and washed with KH. The effect of removing 2-APB on atrial mechanical function was recorded.

To test whether electrical activity preceded and accompanied these spontaneous mechanical events, left atrial appendages were impaled with glass microelectrodes (see, for example, Huang J. et al., “Restitution properties during ventricular fibrillation in the in situ swine heart” Circulation 110:3161-3167 (2004), which is hereby incorporated by reference for such teaching). Their electrical activity and action potentials then were measured using methods known to practitioners of these arts.

iii. Four Protocols Analyzing the Effect of Altering Superfusate Ion Composition and an Anion Transport Inhibitor on SMA

A prolonged diastolic interval was used here to highlight SMA and to favor the consequences of calcium leak on left atrial mechanical function.

To test whether lowering superfusate sodium affected SMA, 3 groups of left atria (n=6-8 per group) were paced at 0.1 Hz and washed with KH containing 127, 109, or 82 mM sodium. Choline chloride was used to maintain constant ionic strength and constant total chloride of 126 mM.

To test whether lowering superfusate chloride affected SMA, 4 groups of left atria (n=6-8 per group) were paced at 0.1 Hz and washed with KH containing 106, 89, 72, or 30 mM total chloride. Sodium glucuronate was used to maintain constant ionic strength and constant total sodium of 145 mM. Fifteen minutes after washing with modified KH, 15 μM 2-APB was added to the superfusate, and the occurrence of SMA was observed during a subsequent 15 min superfusion.

To test whether an anion transport inhibitor affected SMA, atria (n=7) were paced at 0.1 Hz in KH and exposed to 15 μM 2-APB. Following the appearance of SMA, they were titrated with 100 to 400 μM DIDS for 3 to 5 min at each concentration. Whether SMA occurred at the end of each titration period was recorded.

To determine whether decreasing E_{K} affects SMA, left atria were treated with 0 or 15 μM 2-APB (n=7 per group). Ten minutes later, aliquots of 4M KCl were added to the superfusate to increase potassium incrementally to 14 mM. Basal force of contraction was measured in both groups, and the occurrence of SMA was recorded in 2-APB-treated atria.

iv. Five Protocols Evaluating Whether 2-APB Affects SR Activator Calcium in Intact Atria

The initial potentiated beat following prolonged rest (i.e., the PRP maximum force of contraction) reflects activator calcium available for SR release. The PRP response was used to obtain an indirect measure of left atrial SR calcium under our conditions. A 1 min rest was chosen since forces of contraction occurred with ˜30 sec of rest (data not shown).

Left atria (n=6) were superfused in KH, paced at 0.1 Hz, and subjected to PRP. Superfusion proceeded for another 35 min, and a second PRP was performed. These 2 forces were compared to assure the mechanical stability of our preparations.

Two groups of left atria determined how 2-APB affects the maximum atrial force of contraction under conditions of decreased sodium.

One group (n=8) was superfused in KH, paced at 0.1 Hz, and their maximum force of contraction was measured. These atria were washed with KH containing 82 mM sodium, and a second PRP was performed 23 min later.

A second group (n=8) was paced at 0.1 Hz in KH and subjected to PRP. They were exposed to 22 μM 2-APB and, after the appearance of SMA, were washed with KH containing 22 μM 2-APB and 82 mM sodium. Superfusion continued for 23 min and a second PRP was performed.

Two groups of left atria determined how 2-APB affects maximum force of contraction in the presence of normal sodium. Here, DIDS was used to block SMA in atria superfused in normal KH.

Control 0.1 Hz paced atria (n=8) were subjected to PRP and exposed to 400 μM DIDS, and a second PRP was performed 23 min later.

A group of atria (n=8) were paced at 0.1 Hz in KH and subjected to PRP. They were exposed to 22 μM 2-APB, 400 μM DIDS was added to the superfusate after the appearance of SMA, and a second PRP was performed 23 min later.

v. Statistical Analyses

Data are the mean±SEM. Fisher least protected significance difference test compared 2 means. Two-way repeated measure analysis of variance compared means between different groups. Significance was assigned at P<0.05.

vi. Materials

KH reagents were from Fisher Scientific (Norcross, Ga.). Choline chloride, sodium glucuronate, and DIDS were from Sigma Chemical (St. Louis, Mo.). 2-APB was from Tocris-Cookson (Ellisville, Mo.).

b. Results

i. 2-APB Induces SMEs in Isolated Rat Left Atria

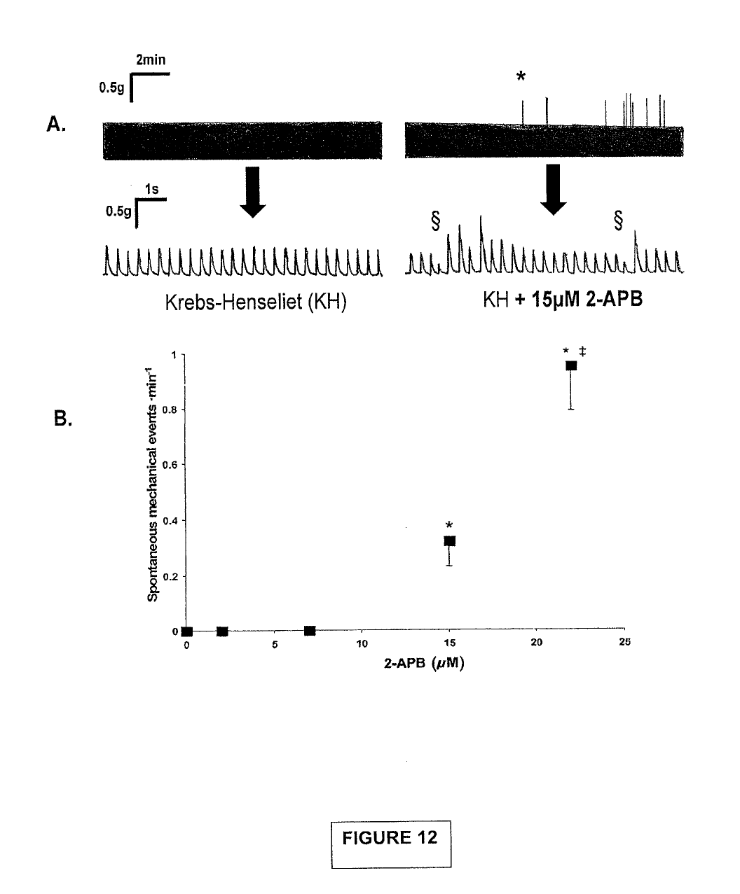

Initial experiments tested whether 2-APB affects rat left atrial electromechanical function. Isolated muscles were superfused and paced at 3 Hz and then exposed to increasing concentrations of 2-APB. All left atria maintained regular mechanical function in the absence of 2-APB. However, intermittent increases in left atrial mechanical force occurred after 5 to 10 min of exposure to >7.5 μM 2-APB. These increases occurred independently of the pacing stimulus (FIG. 12); and their frequency of occurrence increased with 2-APB concentrations >10 μM (FIG. 12).

Next tested was whether this instability in regular atrial electromechanical function occurred in the absence of a pacing stimulus. Untreated left atria were quiescent in the absence of pacing and showed postrest potentiation of mechanical function (FIG. 17A). In contrast, all atria treated with ≧15 μM 2-APB showed numerous SMEs during rest and a blunted post-rest response (FIG. 17B). SMEs persisted during prolonged rest, occurring continuously for at least 45 min after the end of pacing (data not shown).

Impaling left atrial appendages with microelectrodes revealed the action potential characteristics of their myocytes (FIG. 3D; left panel). Left atria undergoing SMA showed spontaneous, normal-looking action potentials (FIG. 3D; right panel). These action potentials occurred in the absence of a pacing stimulus; they preceded and accompanied spontaneous mechanical events in a one-to-one manner. This reveals SMA as an automatic activity.

SMEs may occur as a result of increases in sub-sarcolemmal calcium arising from a diastolic leak of calcium or voltage-independent calcium entry. Thus left atrial pacing rate was lowered to prolong diastole and accentuate any effect that a putative 2-APB-induced calcium leak/entry might have on the stability of regular left atrial mechanical function.

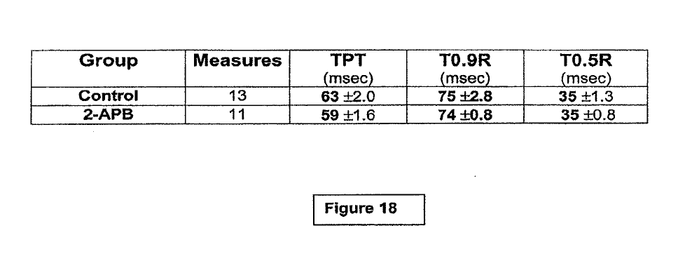

Atria paced at 0.1 Hz showed constant function when superfused in KH, while only few SMEs occurred in slowly paced preparations exposed to ≧10 μM 2-APB (FIGS. 13 A and B). Repeated SMEs occurred in atria exposed to ˜15 μM 2-APB, giving way over time to sustained electromechanical activity that occurred in the absence of the pacing stimulus, SMA (FIG. 13C). Both SMA and the mechanical events that occurred in paced, untreated left atria had similar contraction and relaxation times (FIG. 18).

To evaluate the reversibility of SMA, atria (n=7) were paced at 0.1 Hz, exposed to 15 μM 2-APB, and washed with KH after the appearance of SMA. Removing 2-APB from the superfusate restored regular atrial mechanical function, indicating that SMA is reversible (data not shown).

ii. SMA Depends on the Concentration of Superfusate Sodium, Chloride, and Potassium and is Sensitive to an Anion Transport Inhibitor

It was tested whether altering atrial sodium and chloride gradients affected SMA, as these ions regulate the activity of ion transporters and channels important in initiating ectopic mechanical events. A prolonged diastolic interval was used to highlight the induction or suppression of SMA.

Eight groups of atria were superfused in normal KH, in KH containing decreasing concentrations of sodium at constant chloride and ionic strength, or in KH containing decreasing chloride at constant sodium and ionic strength. All eight groups of left atria were exposed to 15 μM 2-APB for 15 min. SMA occurred in all atria superfused in normal KH (FIG. 9, 10, 11, 15; SMA=100% of the total number of preparations). The number of atria exhibiting SMA decreased as superfusate sodium or chloride decreased, so that regular mechanical function was observed in all left atria superfused either in 82 mM sodium or in 30 mM chloride and exposed to 15 μM 2-APB (FIGS. 9 and 10 respectively).

To test whether an anion transport inhibitor could also suppress SMA, atria were superfused in KH, exposed to 2-APB, and were titrated with DIDS while undergoing SMA. At concentrations >300 μM, DIDS reversed SMA completely, restoring regular atrial mechanical function (FIG. 15).

Finally, we evaluated how superfusate potassium affected SMA. Increasing superfusate potassium from 5.8 mM to ˜9.8 mM reversed SMA and restored regular mechanical function (FIG. 11). The concentrations of potassium required to reverse SMA did not affect basal force of contraction in untreated atria (FIG. 11). Both untreated and 2-APB-treated atria became quiescent at ˜14 mM KCl (FIG. 11).

iii. Evidence that 2-APB May Decrease SR Activator Calcium

Experiments were performed to test whether 2-APB affected the maximum force of atrial contraction, an indicator of the amount of SR activator calcium available for release following depolarization.

The maximum forces of left atrial contraction were not different when measured at the beginning (data not shown) or at the end (FIG. 16) of a 35-min superfusion in KH, low sodium, or DIDS. These maximum forces of atrial contraction also were not different from each other (FIG. 16), results indicating that left atria maintained constant and stable function throughout these protocols.

Acutely decreasing superfusate sodium in the presence of constant 2-APB reversed SMA (FIG. 14A). However, the maximum force of left atrial contraction significantly decreased at the end of superfusion in low sodium and 2-APB when compared with values measured before the addition of 2-APB (FIG. 16). DIDS likewise reversed SMA (FIG. 14B), and the maximum force of atrial contraction also decreased during superfusion in the presence of 2-APB and DIDS compared with values measured before the addition of 2-APB (FIG. 16). Importantly, the maximum force of contraction measured in (2-APB+DIDS)-treated atria was significantly lower than that measured in (2-APB+low Na)-treated atria (FIGS. 16 and 14). Thus 2-APB induced greater loss of maximal mechanical function in DIDS-treated compared with low sodium-treated left atria.

### Example 2

### Confirmation of the Model Against Known Anti-Arrhythmic Agents

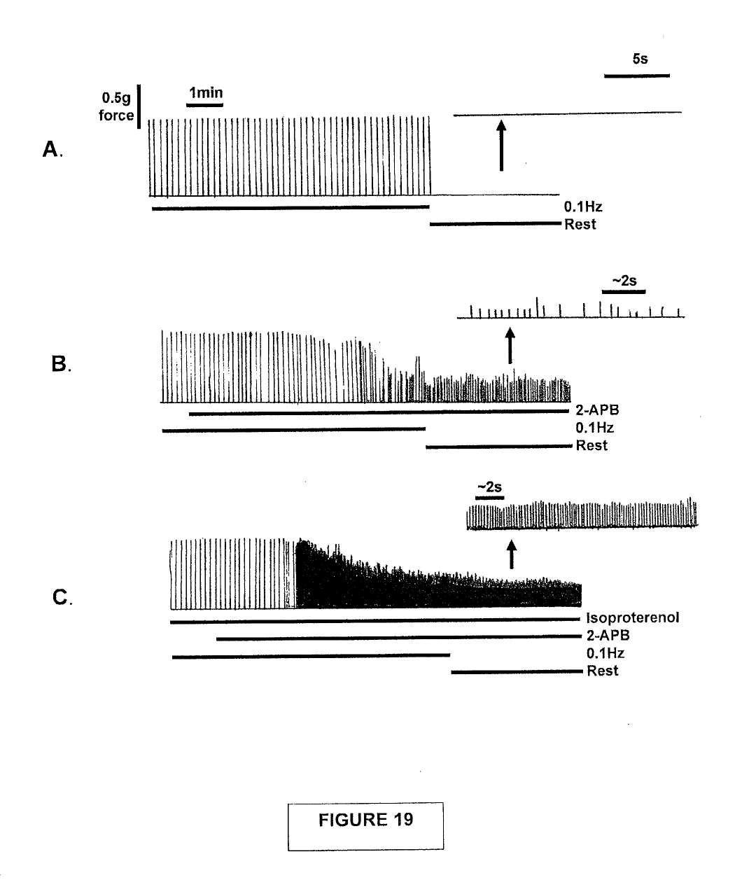

As stated previously, the operation of some embodiments of the model derive from the unexpected discovery that exposing isolated rat left atrial appendage, left ventricular papillary muscles or right ventricular muscle strips to 2-APB and structurally/functionally related compounds induces sporadic ectopic electrical and mechanical events under conditions of normal calcium loading (Wolkowicz et al., J Cardiovascular Pharmacology 49: 325-335 (2007); FIGS. 12 and 17). It was further discovered unexpectedly that, under conditions of increased calcium loading, 2-APB provokes ectopic tachycardic activity in normal rat left atrial appendage and right ventricular muscle strips as exemplars of cardiac muscle (Wolkowicz et al., European J Pharmacology 576: 121-131 (2007); FIG. 19C & see Example 3).

This simple model system provokes sporadic and tachycardic ectopy in normal atria and ventricle, providing a new method to test the anti-arrhythmic properties of pharmaceutical agents.

To confirm the validity of the method, embodiments of the method were tested against several known anti-arrhythmic agents.

Ranolazine is a pharmaceutical that is anti-arrhythmic (see the MERLIN human patient trial, Scirica B. et al. Circulation 116: 1647-1652 (2007)). Ranolazine is sold under the trade name “Ranexa” by CV Therapeutics as an anti-anginal medication. On Jan. 31, 2006, ranolazine was approved for use in the United States by the FDA for the treatment of chronic angina. It was tested whether ranolazine suppresses arrhythmic activity. The recognized target for ranolazine is the persistent sodium current (Saint D A, Persistent (current) in the face of adversity. A new class of cardiac anti-ischemic compounds on the horizon? British J Pharmacology 156:211-213 (2009) which is hereby incorporated by reference for such teaching).

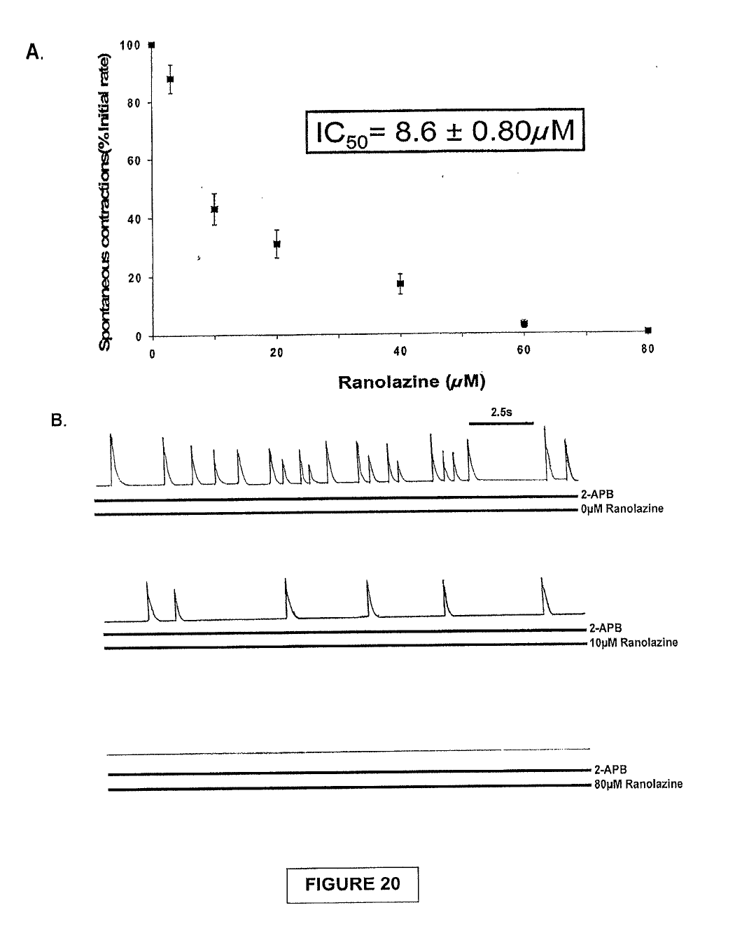

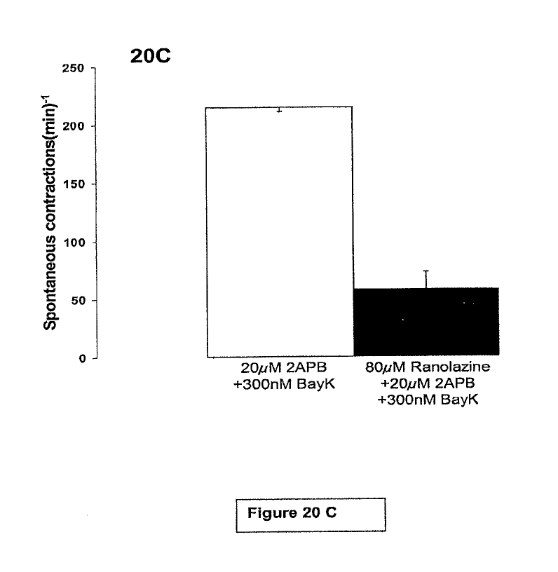

Clinically-relevant concentrations of ranolazine prevent the sporadic ectopic activity in embodiments of the model (FIG. 20A). Ranolazine suppresses, but does not completely prevent, the tachycardic activity that occurs with high calcium loads in this model (FIG. 20B).

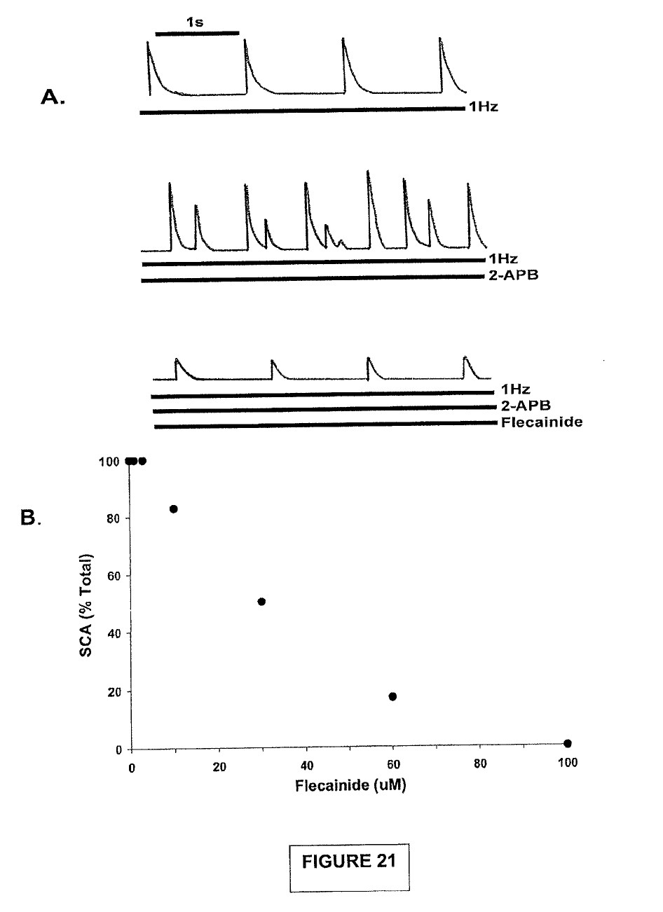

Additional data show that conventional anti-arrhythmic agents like flecainide (FIG. 21) and lidocaine (data not shown) also suppress this arrhythmic activity. Flecainide is an anti-arrhythmic agent sold under the trade names Tambocor, Almarytm, Apocard, Ecrinal, and Flécaine. Lidocaine is a local anesthetic and anti-arrhythmic agent. Both flecainide and lidocaine target persistent sodium current (see Saint D A, Persistent (current) in the face of adversity. A new class of cardiac anti-ischemic compounds on the horizon? British J Pharmacology 156:211-213 (2009) which is hereby incorporated by reference for such teaching).

These data demonstrate that some embodiments of the assay are capable of positively identifying agents that possess anti-arrhythmic activity in vivo. Taken together, these data support this embodiment of the method as a bioassay for conventional, for novel, and for unknown anti-arrhythmic agents.

### Example 3

### A Pharmacological Model for Calcium Overload-Induced Tachycardia in Isolated Rat Left Atria

Few experimental models produce spontaneous tachycardia in normal left atria to allow the study of the cellular mechanisms underlying this contributor to the life-threatening arrhythmias that arise from abnormal automaticity. 2-aminoethoxydiphenyl borate (2-APB) and structurally related compounds provokes SMA and calcium leak in isolated rat left atria. Since calcium leak or entry in the presence of high calcium load may trigger tachyarrhythmias, it was tested how conditions that increase calcium load affect 2-APB-induced ectopic activity. Exposing superfused rat left atria to (i) 30 nM isoproterenol, (ii) 3 μM forskolin, (iii) 300 nM (−) BayK 8644 ((4S)-1,4-Dihydro-2,6-dimethyl-5-nitro-442-(trifluormethyl)phenyl]-3-pyridinecarboxylic acid methyl ester), (iv) 300 nM FPL-64176 (2,5-Dimethyl-442-(phenylmethyl)benzoyl]-1H-pyrrole-3-carboxylic acid methyl ester) or (v) 120 μM ouabain increases their force of contraction, evidence of calcium loading, but does not produce ectopic activity. In the context of this example, the term “left atria” encompasses the use of the left atrial appendage. SMA occurs in left atria superfused with 20 μM 2-APB at 47±6 contractions/min in the absence of pacing. Any of these five agents increase rates of 2-APB-induced SMA to >200 contractions/min in the absence of pacing. Washing tachycardic left atria with superfusate lacking 2-APB restores normal function, demonstrating the reversibility of these effects. Decreasing superfusate sodium and two hyperpolarization-activated current (I_{f}) inhibitors blunt this ectopic activity. Thus conditions that increase atrial calcium load increase the frequency of SMA. Decreasing extracellular sodium and I_{f} inhibitors suppress this spontaneous tachycardia suggesting forward-mode sodium-calcium exchange and I_{f}-like activities underlie this activity. This model may help define cell pathways that initiate tachyarrhythmias and/or fibrillation.

The cellular processes underlying ectopic tachycardia have been rigorously investigated using isolated ventricular myocytes as a model system, but few experimental models are available to evaluate spontaneous tachycardia in intact heart muscle, especially left atria, under well-controlled experimental conditions that do not provoke electrical or structural remodeling. This is important since focal left atrial tachycardia is a recognized cause of atrial fibrillation (Jäis et al., 1997). Moderate concentrations of 2-APB produce sporadic SMA in isolated normal rat left atria, and this ectopic activity may arise from myocyte calcium leak and/or voltage-independent calcium entry (Wolkowicz et al., 2007). Since abnormal myocyte calcium leak or entry in the presence of increased calcium load may generate tachycardic activity in atrium and in ventricle (Tieleman et al., 1997; Pogwizd and Bers, 2004; Tai et al., 2004) it was investigated how conditions that increase atrial calcium load affect 2-APB-induced SMA. Left atria superfused in KH are quiescent in the absence of a pacing stimulus while left atria exposed to ˜20 μM 2-APB produce ˜50 spontaneous contractions per min in the absence of a pacing stimulus Alone, either the β-adrenergic agonist isoproterenol or the adenylyl cyclase activator forskolin significantly increase left atrial force and times of contraction but do not affect left atrial mechanical stability. That is, mechanical contraction requires an external stimulus in these preparations. In contrast, SMA occurs at >200 contractions/min in the presence of 2-APB and either isoproterenol or forskolin; this rate of this spontaneous tachycardia is significantly greater than the spontaneous contraction rate of untreated rat right atria under our experimental conditions, ˜180 contractions/min. Thus activators of β-adrenergic signaling mediate a transition from SMA to spontaneous tachycardic activity (STA) in isolated normal rat left atria exposed to 2-APB. Under these conditions we observe that spontaneous, normal looking action potentials occur and precede all SMEs during STA. No significant decrease in resting membrane potential was noted.

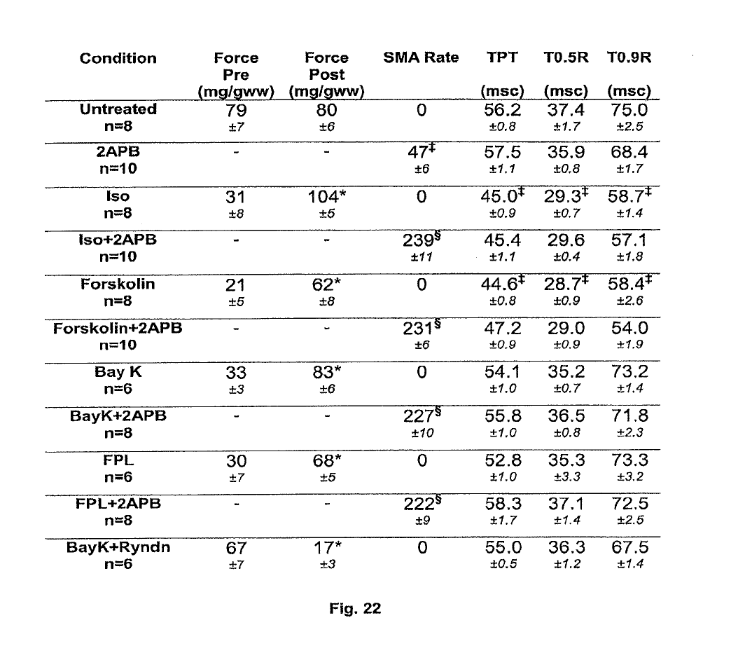

β-adrenergic stimulation increases both myocyte calcium load and calcium flux within myocyte compartments (Kamp and Hall, 2000; Bers, 2002). It was tested whether agents that preferentially increase atrial calcium load initiate STA. Treating left atria with two agents that increase voltage-dependent calcium entry, BayK 8644 and FPL-64176, increases their force of contraction presumably because these structurally unrelated slow channel activators increase slow calcium channel open probability and calcium entry via this channel (Katoh et al., 2000). Importantly, superfusing left atria with 2-APB and either slow calcium channel activator induces STA within 5 to 10 min (FIG. 22). Since left atrial relaxation and contraction times during STA are identical to untreated left atria or to left atria treated with slow calcium channel activators alone, either increased left atrial calcium load or increased calcium entry via the slow channel are sufficient to induce tachycardia in the presence of 2-APB.

To differentiate between these two possibilities, rat left atria were treated briefly with a concentration of ouabain sufficient to double their force of contraction (FIG. 22). Rat cardiac muscle responds poorly to ouabain, nonetheless these increases in contractile force are attributed to an increase in heart calcium load that occurs independently of the slow calcium channel (Illanes and Marshall, 1964; Vassalle and Lin, 2004). Short-term exposure to ouabain alone does not affect rat left atrial mechanical stability; importantly, 2-APB and ouabain induce STA at rates similar to those measured under the preceding four conditions (FIG. 22). This suggests that an increase in left atrial calcium load in the presence of 2-APB is sufficient to produce STA.

Removing 2-APB from the superfusate suppresses STA suggesting that the cell mechanisms underlying STA are reversible. Whether prolonged periods of STA change atrial protein function or gene expression to favor remodeling requires further analyses (Tavi et al., 2003; Carnes et al., 2001).

One complication of these results is the fact that rat myocyte calcium handling during excitation-contraction coupling differs from other species; it depends little on sarcolemmal calcium flux (Bers, 2002). In addition, rat sarcoplasmic reticulum has relatively high calcium content under normal conditions (Satoh et al., 1997).

While BayK 8644 and FPL-64176 both increase slow calcium channel open probability, BayK 8644 also interacts with the ryanodine receptor to initiate sarcoplasmic reticulum calcium leak (Katoh et al., 2000); thus ryanodine receptor calcium leak might contribute to this novel tachycardia. This is important as prior art suggests ryanodine receptor calcium leak may elicit ventricular ectopic and tachycardic activity (Marx et al., 2000, Ai et al., 2005). However, as both BayK 8644 and FPL-64176 induce left atrial STA, any effect of the former agent on atrial sarcoplasmic reticulum leakiness may not be important here. In support of this contention, ryanodine decreases left atrial force of contraction, evidence for sarcoplasmic reticulum calcium depletion (Sutko et al., 1997), but ryanodine does not affect left atrial contraction frequency measured in the presence or absence of BayK 8644 (FIG. 22).

While a cellular site of action for ryanodine is clearly resolved, how 2-APB influences atrial calcium homeostasis to trigger ectopic activity is less clear. In this regard Maruyama et al., (1997,) and Ma et al., (2002), among others (Bootman, 2000), show that low concentrations of 2-APB block IP3 receptor calcium release while moderate concentrations induce leakage of calcium from intracellular stores. Importantly, Ma et al., (2002) reports that 2-APB does not induce calcium leakage in cells lacking IP3 receptors, suggesting that these stores may be the source of leaked calcium proposed to occur under our experimental conditions. In addition, prior art shows that 2-APB induces cell calcium entry via the SOC over the concentration range which elicits SMA and STA. Thus 2-APB may affect multiple sites in voltage-independent calcium signaling to provoke automatic action potentials, including but not limited to the IP3R and the SOC.

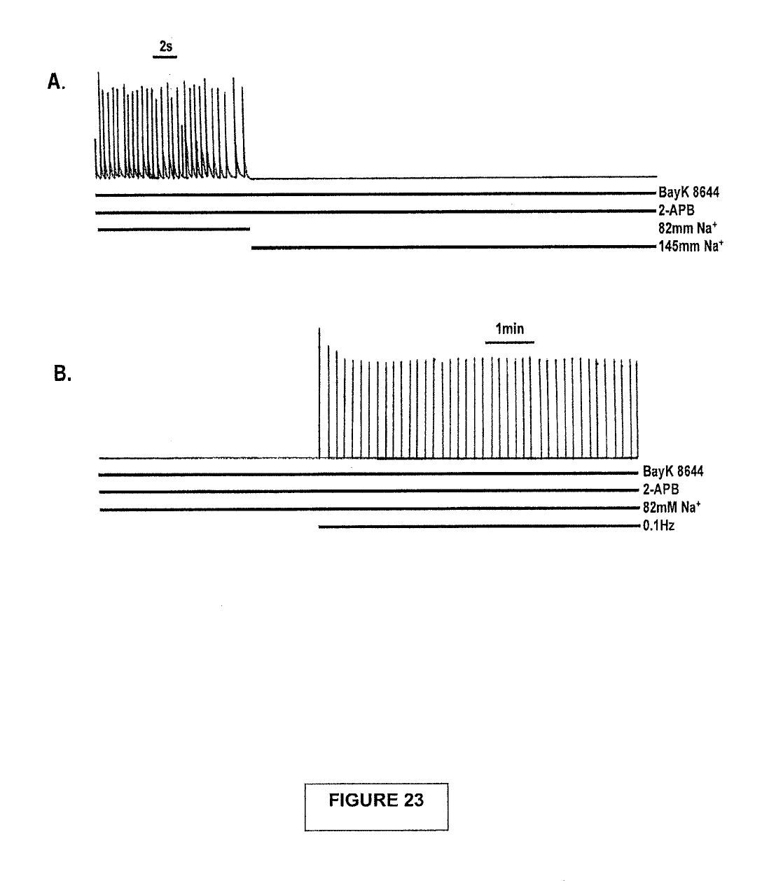

Lowering extracellular sodium suppresses or abolishes phase 4 depolarization in sinoatrial node cells, indicating a role for forward-mode NCX in sinoatrial pacemaker activity (Sanders et al., 2006). The data described below show lowering superfusate sodium also suppresses 2-APB-induced left atrial STA promptly and completely (FIG. 23) perhaps by decreasing the driving force for forward-mode NCX. The ability of an external stimulus to recapture these left atria exposed to low sodium (FIG. 23) indicates that excitation-contraction coupling remains intact in these isolated muscles. However, lowering extracellular sodium affects multiple ion transport processes that may impact our results; for example, sodium-proton and sodium-bicarbonate exchange may be suppressed here. To critically test whether other consequences of decreased extracellular sodium contributed to the results requires future measurement of atrial pH and resting membrane potential under our conditions. In addition, alterations in sodium current characteristics may contribute both to the ectopic activity described here and to its sodium sensitivity (Belardinelli et al., 2006).

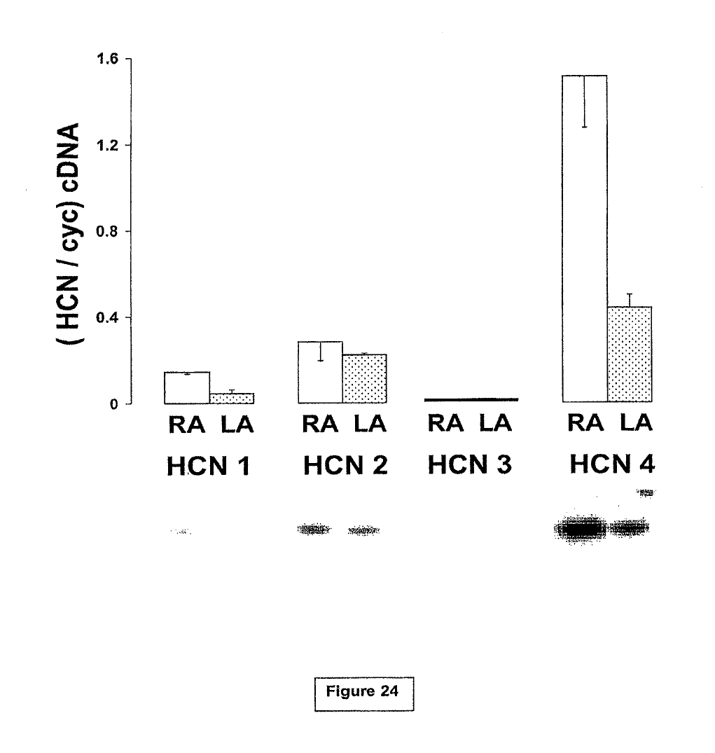

While I_{f}-like activity and HCNs occur in non-sinoatrial myocardium (Carmeliet, 1984; Cerbai and Mugelli, 2006; FIG. 24), previous analyses show that these I_{f} activate outside of a physiologically relevant range of membrane potentials. Nonetheless, activation of non-sinoatrial I_{f} is hypothesized to contribute to ectopic activity (Zorn-Pauly et al., 2004; Cerbai and Mugelli, 2006). Indeed, adenovirus-driven overexpression of HCN2 in dog left atrium produces spontaneous in vivo depolarizations that offer proof-of-principle for this possibility, (Qu, 2003).

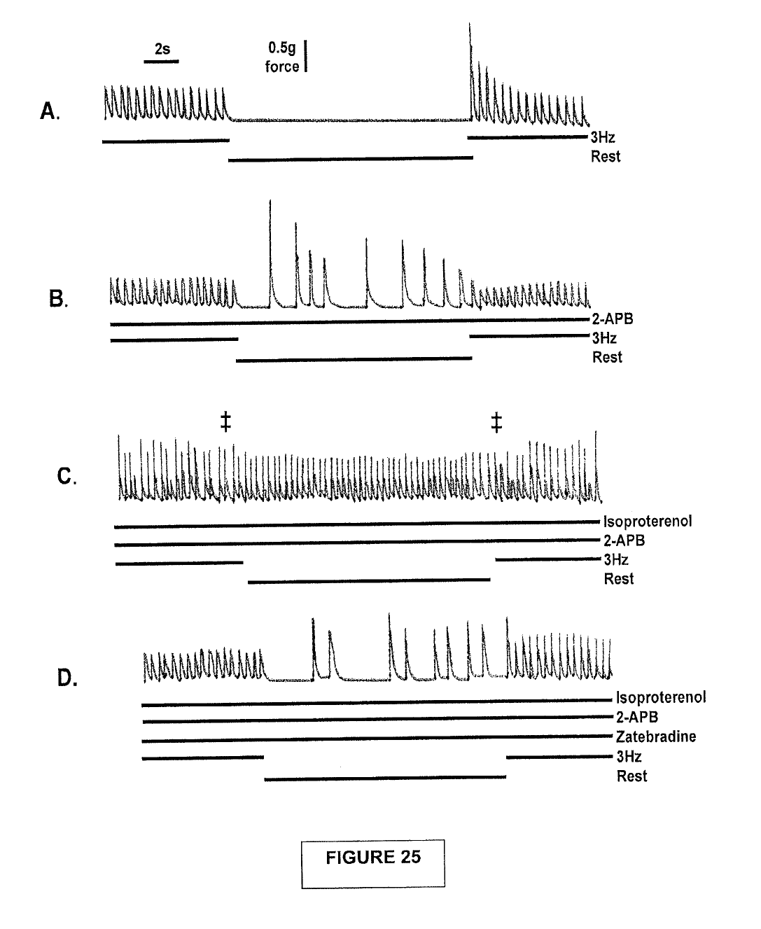

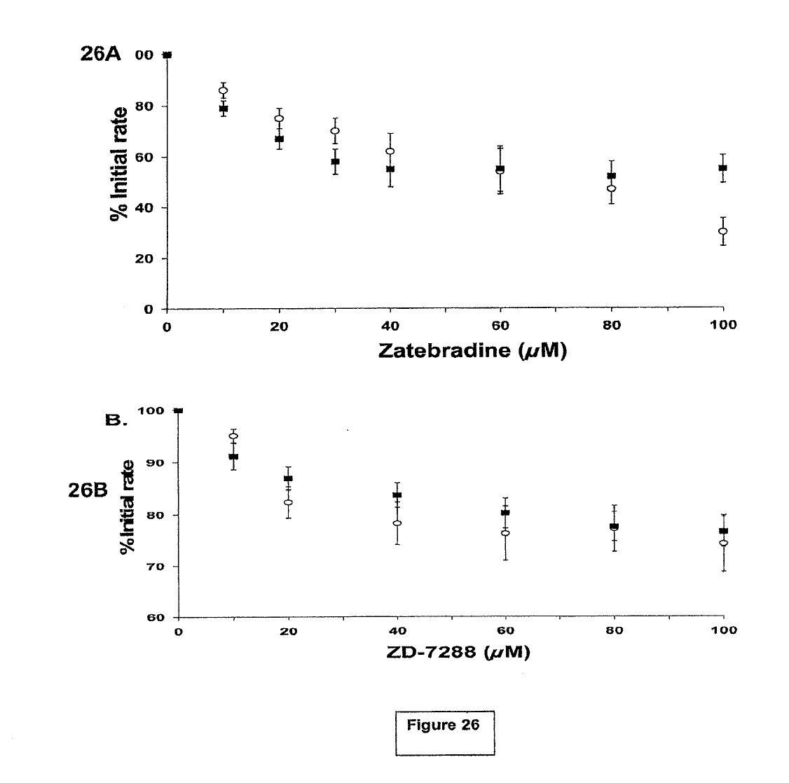

The data described below support the possibility that an endogenous I_{f}-like activity contributes to 2-APB-induced STA as zatebradine and ZD-7288, two structurally unrelated I_{f} blockers, suppress this response (FIGS. 25 and 26). The left atrial I_{f}-like activity observed here appears to be pharmacologically distinct from sinoatrial I_{f}. Specifically, zatebradine inhibits left atrial STA linearly with an IC₅₀ greater than values reported for guinea pig right atria (Perez et al., 1995) or measured in our rat right atria (FIG. 25A). ZD-7288 which binds to the I_{f} channel at a site distinct from zatebradine (Baruscotti et al., 2005) decreases left atrial STA and right atrial spontaneous rates of contraction to a similar degree (FIG. 25B); the latter effect is comparable to that reported in rat right atrial myocytes (Sanders et al., 2006). Furthermore, in contrast to right atrial pacemaker activity where cAMP is required for maximum contraction frequencies, increased calcium load, under conditions that do not affect cyclic nucleotide metabolism (i.e., BayK 8644 or FPL-64176; Katoh et al., 2000), activate left atrial STA maximally. This suggests that cell calcium influences left atrial I_{f}-like activity. Differences in right and left atrial HCN isoform expression (Baruscotti et al., 2005; Cerbai and Mugelli, 2006; FIG. 25), differences in HCN accessory protein expression and post-translational HCN modification, or the expression of HCN splice variants in non-sinoatrial myocardium may underlie the pharmacological and functional differences between right and left atrial spontaneous activity.

Clinical and experimental data indicate that multiple sites of focal tachycardia arise within diseased left atrium including regions near the pulmonary veins and regions of left atrial muscle itself (Jäis et al. 1997; Shah, 2004; Nattel, 2005; Rostock et al., 2006). While reentrant activity may contribute to these clinical observations in diseased atrium (Nattel, 2002), the data described below show that normal left atrial muscle can initiate and sustain spontaneous tachycardia under specific and controllable experimental conditions. Thus, this pharmacological model may help define some of the cell mechanisms responsible for ectopic atrial tachycardia.

a. Materials and Methods

These investigations conform to the Guide for the Care and Use of Laboratory Animals published by the US National Institutes of Health (NIH Publication No. 85-23, revised 1996).

i. Isolation and Superfusion of Rat Left and Right Atria, and the Preparation of 2-APB

Male Sprague-Dawley rats (325-400 g) were anesthetized with intra-peritoneal pentobarbital (0.1 g/kg). Their right and left atria were isolated, superfused at 30° C. in Krebs-Henseleit buffer, and nearly isometric forces of contraction were measured from these muscles (Wolkowicz et al., 2002). 2-APB was prepared as a 150 mM dimethyl sulfoxide stock. Dimethyl sulfoxide did not affect atrial mechanical function.

ii. Effect of β-Adrenergic Signaling Activators on SMA

Left atria (n=8 per group) were paced at 3 Hz and (i) titrated with 0 to 30 nM isoproterenol (Ahlquist, 1970) or (ii) exposed to 3 μM forskolin (Laurenza et al., 1980). Left atrial force and frequency of contraction, the time to peak tension and relaxation times were recorded after a 3-5 min exposure to forskolin or to any concentration of isoproterenol. The 3 Hz pacing stimulus was interrupted at the end of these protocols to measure SMEs.

Left atria (n=10 per group) were paced at 0.1 Hz and treated with (i) 20 μM 2-APB for 10 min, (ii) with 30 nM isoproterenol for 5 min followed by 20 μM 2-APB for 10 min, and (iii) with 3 μM forskolin for 5 min followed by 20 μM 2-APB for 10 min.

Left atrial contraction and relaxation times were recorded at these five times, the pacing stimulus was stopped and rates of SMA were recorded. To determine whether initial pacing rate affects left atrial response to 2-APB and isoproterenol, left atria (n=5-7 per group) were paced at 3 Hz and (i) remained untreated for 15 min, (ii) were treated with 20 μM 2-APB alone for 10 min, or (ii) were treated with 30 nM isoproterenol for 5 min followed by 20 μM 2-APB for 10 min. Atrial mechanical function was measured at these four times; the pacing stimulus then was stopped and rates of SMA were recorded.

iii. Effect of Slow Calcium Channel Activators on SMA

Left atria (n=6 per group) were paced at 3 Hz, titrated with 0 to 300 nM (−)BayK 8644 ((45)-1,4-Dihydro-2,6-dimethyl-5-nitro-4-[2-(trifluormethyl)phenyl]-3-pyridinecarboxylic acid methyl ester; Franckowiak et al. 1985) or FPL-64176 ((2,5-Dimethyl-4-[2-(phenylmethyl)benzoyl]-111-pyrrole-3-carboxylic acid methyl ester; Zheng et al., 1991) and their mechanical function and rates of SMA were recorded after a 5 min exposure to any concentration of slow calcium channel activator.

Left atria (n=8 per group) were paced at 0.1 Hz, exposed to 300 nM BayK 8644 or FPL-64176 for 5 min followed by 20 μM 2-APB for 10 min. Atrial mechanical function was recorded at this time; the pacing stimulus then was stopped and rates of SMA were recorded.

To test whether ryanodine receptor calcium leak produces SMA, left atria (n=6 per group) were paced at 0.1 Hz, treated with 0 or 300 nM BayK 8644 for 5 min, and then exposed to 600 nM ryanodine (Sutko et al., 1997). Atrial mechanical function and rates of SMA were recorded 20 min after exposure to ryanodine.

To evaluate the reversibility of the SMA that occurs in the presence of 2-APB and BayK 8644, left atria (n=6) were paced at 0.1 Hz and exposed to 300 nM BayK 8644 for 5 min and then to 20 μM 2-APB. One minute after the appearance of SMA these left atria were washed with 300 ml (10 bath volumes) of Krebs-Henseleit containing 300 nM BayK 8644. Rates of SMA were measured 5 min later.

iv. Effect of Ouabain on SMA

Left atria (n=7) were paced at 3 Hz, treated with 120 μM ouabain (Illanes and Marshall, 1964), and any change in mechanical function was recorded at a new steady state, ˜10 min later. These atria were exposed to 20 μM 2-APB and their mechanical function and rates of SMA were recorded 10 min later.

v. The Sodium Sensitivity of the SMA Occurring in the Presence of 2-APB and BayK 8644

Rat left atria (n=9) were paced at 0.1 Hz and exposed to 300 nM BayK 8644 and 20 μM 2-APB. Ten min later the pacing stimulus was stopped and 3 min later superfusate sodium was rapidly lowered to 82 mM (Wolkowicz et al., 2007). Changes in the rate of SMA were recorded. Three to 5 min after lowering sodium the 0.1 Hz pacing stimulus was reapplied to recapture these left atria. Superfused rat right atria (n=7) beat at their intrinsic rate in Krebs-Henseleit superfusate for 15 min and superfusate sodium then was rapidly lowered to 82 mM. Right atrial contraction rate was measured at a new steady state, 3 min later.

vi. The I_{f} Inhibitor Sensitivity of the SMA Occurring in the Presence of 2-APB and BayK 8644

3 Hz-paced left atria (n=9 per group) were superfused (i) in Krebs-Henseleit alone or (ii) they were treated with 20 μM 2-APB for 10 min, (iii) with 300 nM BayK 8644 for 10 min and with 20 μM 2-APB for 10 min, or (iv) they were treated with 300 nM BayK 8644 and 20 μM 2-APB for 10 min, then with 70 μM zatebradine (3-[3-[[2-(3,4-Dimethoxyphenyl)ethyl]methylamino] propyl]-1,3,4,5-tetrahydro-7,8,-dimethoxy-2H-3-benzazepin-2-one hydrochloride) for 10 min (Baruscotti et al., 2005). Pacing was interrupted at these times and rates of SMA were recorded.

Left atria (n=9) were paced at 0.1 Hz, treated with 300 nM BayK 8644 for 5 min, then with 20 μM 2-APB for 10 min, and the pacing stimulus was stopped. These preparations then were incubated with six increasing concentrations of zatebradine (10-100 μM) for 3-5 min at each concentration, and rates of SMA were recorded. Untreated right atria (n=9) also were titrated with zatebradine and its effect on contraction frequency was recorded.

To test whether zatebradine affects left atrial force of contraction, two groups of left atria (n=9 per group) were paced at 3 Hz and treated with 300 nM BayK 8644. Five minutes later one group was incubated with six increasing concentrations of zatebradine (10-100 μM) for 3-5 min at each concentration, and forces of contraction were recorded. A second group was treated with BayK 8644 but not zatebradine, and forces of contraction were recorded at corresponding times.

To test whether zatebradine affects the SMA occurring in the presence of 2-APB and isoproterenol, left atria (n=7) were paced at 0.1 Hz, treated with 30 nM isoproterenol for 5 min, then with 20 μM 2-APB for 10 min, and the pacing stimulus was stopped. Rates of SMA were recorded, these left atria were exposed to 70 μM zatebradine for 10 min, and rates of SMA were recorded again. A group of right atria (n=7) also were exposed to 30 nM isoproterenol for 5 min and then to 70 μM zatebradine for 10 min. Right atrial contraction frequency was recorded at these two times.

To test whether ZD-7288 (4-Ethylphenyl amino-1,2-dimethyl-6-methylaminopyridinium chloride), an I_{f} inhibitor structurally unrelated to zatebradine (Baruscotti et al., 2005; Sanders et al., 2006) affects the SMA occurring in the presence of 2-APB and BayK 8644, rat left atria (n=7) were treated with 300 nM BayK 8644 for 5 min, then with 20 μM 2-APB for 10 min, and the rates of SMA were recorded. These atria then were incubated with six increasing concentrations of ZD-7288 (10-100 μM) for 3-5 min at each concentration, and the rates of SMA were recorded. Right atria (n=7) were titrated similarly with ZD-7288 (0-100 μM) and the change in their contraction frequency was recorded.

vii. RT-PCR Analysis of Right and Left Atrial Hyperpolarization-Activated Cyclic Nucleotide Gated Cation Channel (HCN) mRNAs

Total RNA was isolated from rat left and right atria (n=3 per) using QiaShredder and RNeasy kits. cDNA was transcribed from 1 μg of total left and right atrial RNA using random primers and 200 U of reverse transcriptase (Wolkowicz et al., 2004). HCN primers were obtained from the sequences for rat HCN 1 (GenBank accession no. NM053375), rat HCN2 (NM053684), rat HCN3 (NM053685), and rat HCN4 (NMO21658). Rat cyclophilin was used as an internal control (Wolkowicz et al., 2004). All HCN amplifications were performed using a MJ PTC200 Thermal Cycler (BioRad, 226 Hercules, Calif.) in 50 μl of Taq PCR Master Mix containing 100 ng of cDNA. Amplifications employed 28 cycles of (i) a 1 min 90° C. denaturing step, (ii) a 45 s 55° C. annealing step, and (iii) a 45 s 72° C. amplification step, and a final 3 min product extension at 72° C. Aliquots of these reactions were electrophoresed through 1.2% agarose gels and analyzed using a Kodak Gel Logic100 imaging system. The intensity of atrial HCN and cyclophilin cDNAs were quantitated using Kodak Molecular Imaging Software.

viii. Statistical Analyses

Data are the mean±S.E.M. Fisher's least protected significance difference test compared two means. Two-way repeated measure analysis of variance compared means between different groups. Significance was assigned at P<0.05.

ix. Materials

Krebs-Henseleit reagents were from Fisher Scientific (Norcross Ga.). (−)BayK 8644, FPL-64176, zatebradine, ZD-7288, ryanodine, and 2-APB were from Tocris-Cookson (Ellisville, Mo.). Forskolin, isoproterenol, and ouabain were from Sigma Chemical (St. Louis, Mo.). QiaShredder, RNeasy RNA isolation kits, and Taq PCR Master Mix were from Qiagen (Valencia, Calif.). Maloney murine leukemia virus reverse transcriptase was from Invitrogen (Carlsbad, Calif.). Oligonucleotides were from MWG Biotech (High Point, N.C.).

b. Results

i. Activators of β-Adrenergic Signaling Increase the Frequency of Left Atrial SMA

Isoproterenol and forskolin increase left atrial force of contraction (FIG. 22), and decrease left atrial time to peak tension and atrial relaxation times (FIG. 22: cp. Untreated, Iso & Frsk; TPT, T0.5R & TO.9R). Left atria treated with these activators of the β-adrenergic signaling cascade are quiescent in the absence of pacing (FIGS. 19 and 22).

2-APB (20 μM) produces SMA in rat left atria at 47±6 contractions/min in the absence of pacing (FIGS. 19 and 22). The contraction and relaxation times of the contractile events that occur during SMA are similar to values measured in untreated left atria (FIG. 22). Remarkably, SMA occurs at a frequency of 239±11 and 231±5 contractions/min in the presence of 2-APB and isoproterenol or forskolin, respectively (FIGS. 19 and 22); this high-frequency ectopic activity was designated spontaneous tachycardic activity (STA). The time to peak tension and the relaxation times measured in left atria undergoing STA are similar to those measured in atria treated with isoproterenol or forskolin alone (FIG. 22). Hence activators of β-adrenergic signaling markedly increase the frequency of left atrial SMA. This suggests that one or more of the targets or the consequences of adrenergic signaling initiate and maintain STA.

STA does not depend on the initial rate of atrial pacing. Specifically, STA arose immediately following the termination of 3 Hz pacing in left atria that were exposed to isoproterenol and 2-APB (FIG. 23). Mechanical discordance occurs in [isoproterenol+2-APB]-treated left atria when paced at 3 Hz and dissipates in the absence of pacing (FIG. 23: cp. 3 Hz & Rest).

ii. Slow Calcium Channel Activators Induce STA

BayK 8644 and FPL-64176 increase left atrial force of contraction (FIG. 22) without affecting the time to peak tension or left atrial relaxation times (FIG. 22), indicating that left atrial calcium loading takes place here. SMA or SMEs do not occur in left atria treated with either slow calcium channel activator alone (FIG. 22).

STA occurs at 227±10 and at 222±9 contractions/min in left atria treated with 2-APB and 300 nM BayK 8644 or FPL-64176 (FIG. 22). These rates are not different from those measured in left atria treated with 2-APB and isoproterenol or forskolin. However, time to peak atrial tension and the relaxation times measured in left atria treated with 2-APB and either slow calcium channel activator are similar to those measured in untreated preparations (FIG. 22). Thus, appendage STA can occur in the absence of any significant change in contraction or relaxation time.

Washing spontaneously tachycardic left atria with superfusate containing slow calcium channel activator but no 2-APB completely reverses STA (224±12 vs. 0.7±0.4 contractions/min, Pre- and Post-washing, respectively). This suggests that a short-term exposure to the experimental conditions that elicit STA does not disrupt left atrial integrity.

Next was tested whether ryanodine receptor calcium leakage could produce STA. Time-dependent decreases in force of contraction occur in left atria exposed either to 600 nM ryanodine or to BayK 8644 and ryanodine, indicating leakage of sarcoplasmic reticulum activator calcium (FIG. 22). However, SMEs, SMA or STA does not occur under these conditions (FIG. 22). This suggests that while ryanodine receptor calcium leak depletes sarcoplasmic reticulum calcium stores and may trigger ectopic events under some conditions (Marx et al. 2000; Ai et al., 2005), it does not contribute to the activity reported here.

iii. Ouabain Induces STA

Short-term exposure of superfused 3 Hz-paced rat left atria to 120 μM ouabain doubles their force of contraction, an indication of calcium loading (Vassalle and Lin, 2004), but does not affect their mechanical stability (FIG. 22). A subsequent 5-10 min exposure to 2-APB produces STA at 202±5.6 contractions/min (FIG. 22). Similar time to peak tension and left atrial relaxation times occur in ouabain- and (ouabain+2-APB)-treated left atria, and are comparable to untreated preparations (FIG. 22).

Thus STA occurs with five experimental conditions that increase calcium load via distinct mechanisms; β-adrenergic signaling, slow calcium channel activation, and ouabain inotropy (Kamp and Hall 2000; Kutch et al., 2000; Vassalle and Lin, 2004).

iv. STA is Sensitive to Superfusate Sodium

Rapidly lowering superfusate sodium from 145 to 82 mM abruptly suppresses left atrial STA and left atria remain quiescent under these conditions (FIG. 23). Excitation-contraction coupling remains functionally intact in these preparations as a 0.1 Hz pacing stimulus recaptures them (FIG. 23). Rat right atrial contraction frequency also decreases significantly when superfusate sodium is reduced to a similar extent (145 mM Na=163±11.8 vs. 82 mM Na=65±7.1 contractions/min) (Ju and Allen, 1998; Hünser et al., 2000; Sanders et al., 2006).

v. I_{f} Inhibitors Suppress STA

Zatebradine (70 μM) significantly decreases both the rate of STA in left atria treated with BayK 8644, and 2-APB (FIG. 25) and the mechanical discordance that occurs in 3 Hz-paced left atria treated with BayK 8644 and 2-APB (FIG. 25). Zatebradine decreases STA linearly with an IC₅₀ of 58±5 μM in intact left atria exposed to BayK 8644 and 2-APB (FIG. 26). This I_{f} inhibitor also decreases the rate of appendage STA in left atria treated with isoproterenol and 2-APB (225±3.6 vs. 110±24.5 contractions/min; (isoproterenol+2-APB) vs. (isoproterenol+2-APB+70 μM zatebradine), respectively). Zatebradine decreases right atrial contraction frequency to a maximum of ˜50% with an IC₅₀ of 12±1.1 μM (FIG. 26), values similar to those reported for guinea pig right atria (Perez et al., 1995). Isoproterenol increases right atrial contraction frequency (177±4.7 vs. 276±11.4 contractions/min; 0 vs. 30 nM isoproterenol, respectively) and zatebradine depresses right atrial contraction frequency under these conditions as well (212±9.7 contractions/min; (isoproterenol+70 μM zatebradine)).

Zatebradine is structurally related to the slow calcium channel antagonist verapamil (Doerr and Traunvein, 1990). To assure that zatebradine does not suppress left atrial slow channel activity under our conditions, we measured the force of contraction of left atria that were treated with BayK 8644 and then (i) titrated with 10-100 μM zatebradine or (ii) were left untreated. Zatebradine does not affect left atrial force of contraction under these conditions suggesting that it does not inhibit slow calcium channel activity across this range of concentrations (Untreated=97±1.5% vs. Zatebradine-treated=95±3.8% of initial force).

ZD-7288, an I_{f} inhibitor structurally unrelated to zatebradine, also decreases left atrial STA initiated by BayK 8644 and 2-APB (FIG. 26B: ▪). As expected (Sanders et al., 2006), ZD-7288 decreases right atrial contraction frequency less robustly than zatebradine (FIG. 26).

vi. Rat Left Atria Contain HCN mRNAs

Rat left atria contain HCN2 mRNA in amounts similar to those measured in rat right atria (FIG. 24) and HCN4 mRNA in lower amounts than those in right atria (FIG. 24). Rat left atria contain no detectable HCN3 (FIG. 24) and little HCN I mRNA (FIG. 24).

### Example 4

### Ventricular Muscle Models of SMA and STA

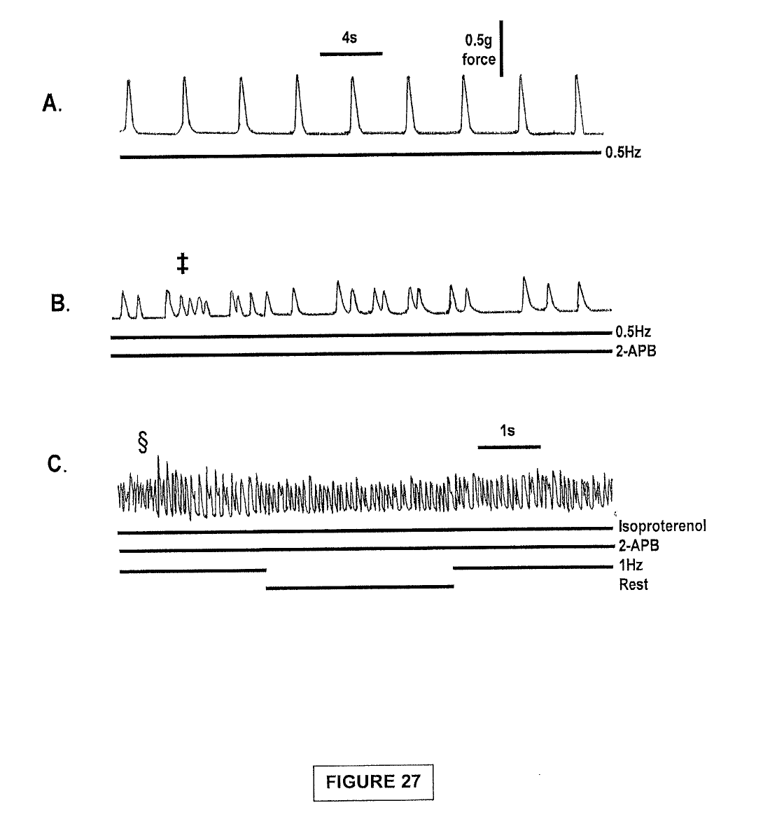

To further establish this model as a general test for arrhythmic and anti-arrhythmic agents, right ventricular muscle strips and right or left ventricular papillary muscles were isolated from anesthetized rats as cardiac muscle exemplars using methods known to persons skilled in the art. Additional exemplars could include ventricular or atrial conduction system cells and the SAN itself. These muscles were then superfused as described above and paced at 0.5 Hz. These muscles show normal excitation-contraction coupling and produce mechanical force only when stimulated (FIG. 27A). When 2-APB or a structurally or functionally related compound such as diphenyl boronic anhydride (DPBA) was added to the superfusate in appropriate amounts, ventricular SMA was observed (FIG. 27B). This ventricular muscle showed STA, similar to atrial muscle, when it is exposed to an agent that enhances heart muscle calcium stores (FIG. 27C).

Thus all non-automatic cardiac muscle exemplars tested shows the ability to express both sporadic and tachycardic electro-mechanical activity under the experimental conditions described herein. As it has been shown that atria and ventricle contain distinct ion channel characteristics, the model described here can be used to identify anti-arrhythmic agents that may be potentially specific to atrial muscle, to ventricular muscle, or to both.

### Example 5

### Model Simulating Fibrillation

This example demonstrates that the model systems described may be used to screen for agents that suppress atrial or ventricular fibrillation and tachycardia. The example confirms that tachycardia and fibrillations can be simulated using the model.

It has been observed that bioassay temperature is preferably increased to 37° C. This is advantageous due to the difference in the temperature profile between normal sinoatrial node activity and SMA/STA.

To determine the relationship between temperature and rates of STA, rat left atrial appendage (n=6-8) were superfused at 30° C., paced at 0.1 Hz, treated for 5 min with 300 nM (−) BayK 8644 and then treated for 10 min with 22 μM 2-APB. Pacing was stopped and rates of STA were recorded. A second group of appendages (n=8) were superfused at 23° C. and treated identically to the first. After 10 min STA was measured and bath temperature was increased to 37° C. Maximum contraction rates were measured over 10 min.

Unpaced rat right atria (n=8) were superfused at 30° C. and rates of SAN pacemaker-driven mechanical contraction were recorded. A second group of right atria was superfused at 23° C. and their contraction rates were recorded; muscle bath temperature was increased to 37° C. and maximum rates of spontaneous contraction were measured over 10 min.

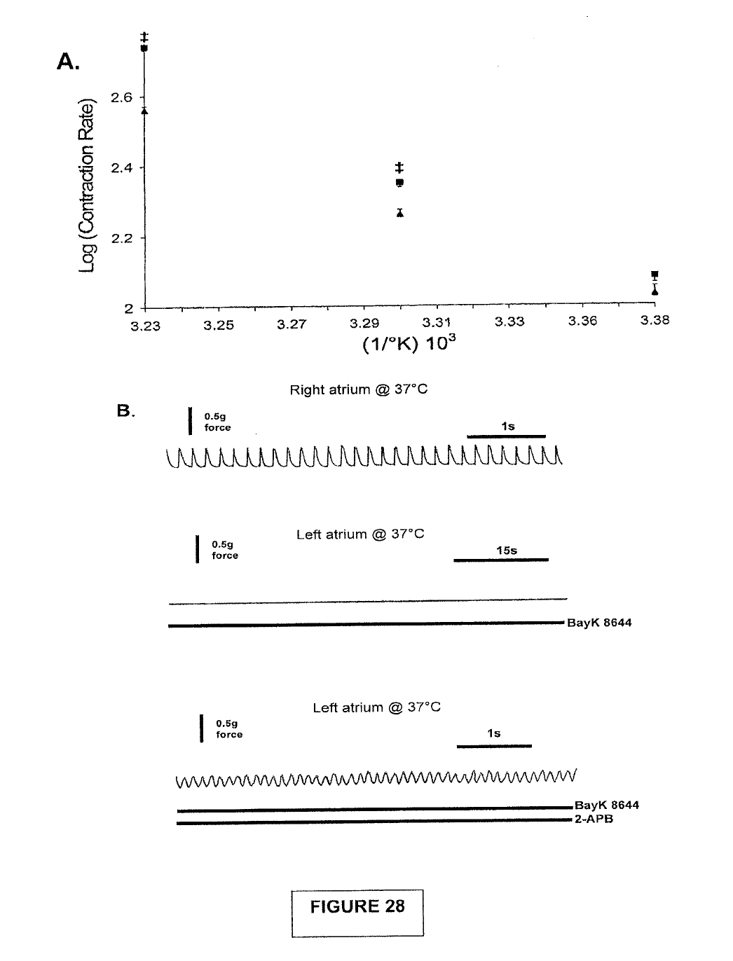

As shown in FIG. 28A, a predictable relationship was discovered between temperature and left atrial STA and right atrial contraction rate. FIG. 28B shows mechanical function of isolated cardiac muscle at 37° C. The upper graph shows mechanical function of a superfused rat right atrium measured without pacing at 37° C. The middle graph shows mechanical function of a 0.1 Hz-paced, superfused left atrial appendage treated for 5 min with 300 nM (−)BayK 8644 alone at 37° C. without pacing. The lower graph shows STA of a rat left atrial appendage superfused at 37° C. and measured without pacing.

While fibrillation-like results can be obtained at 30° C., increasing the bio-assay temperature to 37° C. increases the influence this ectopic activity has over the electromechanical instability of isolated heart muscle.

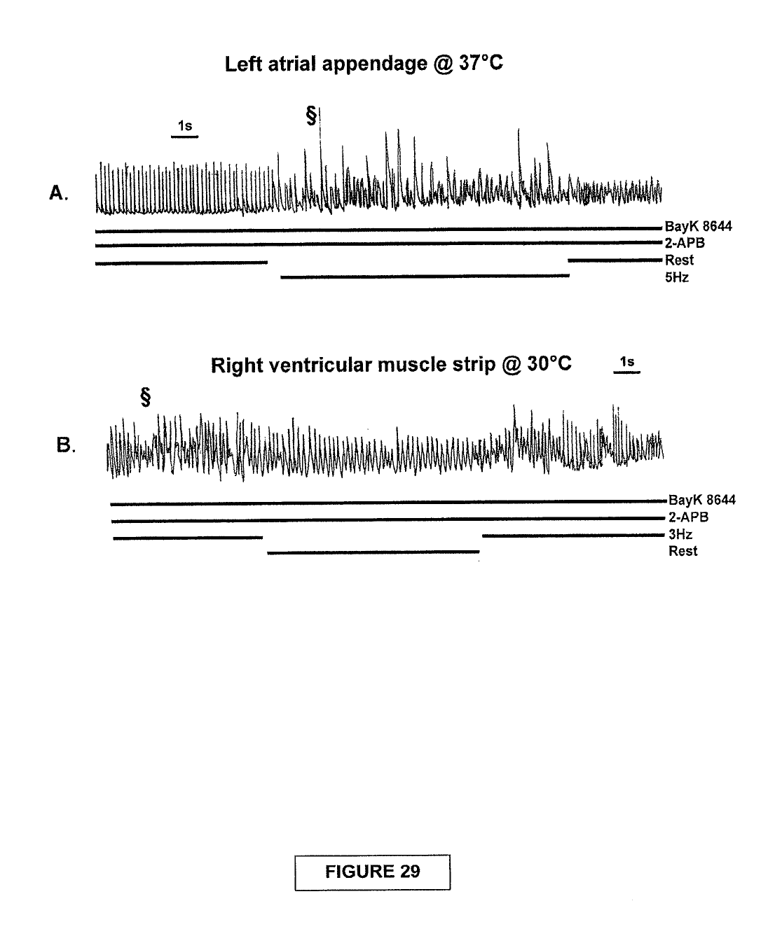

Clinical evidence suggests that human atrial and ventricular fibrillation arise when one or more ectopic tachycardic foci continually discharge and disrupt normal heart electromechanical function driven by the sinoatrial node. Further experiments demonstrated that the model recapitulates this scenario in isolated atrial or ventricular muscle. Specifically, isolated, superfused heart muscle, atrial or ventricular, treated with an appropriate concentration of 2-APB (>20 μM) and with an agent that increases myocyte calcium load was exposed to external pacing that stimulates the muscle at a physiological rate and with strength just sufficient to capture it. Under these conditions isolated atrial and ventricular muscle produced disorganized, fibrillation-like mechanical activity (FIG. 29). In the absence of external pacing, these atrial and ventricular muscles exhibited only STA (FIGS. 29 and 27, Rest).

This indicates the persistence of the underlying arrhythmic principle which is responsible for the fibrillation-like activity in embodiments of this model.

Thus, the above experiments establish the operation of one embodiment of this bioassay as follows: 1) Isolated left atrial appendage or ventricular muscle superfused or perfused in Krebs-Henseliet perfusate at a physiological temperature paced initially at a slow sub-physiological rate; 2) Exposure of this muscle to >20 μM of 2-APB or a structurally- or functionally-related compound; 3) Exposure of this muscle to a condition that increases its internal calcium either prior to step (2) or following the appearance of ectopy in the presence of 2-APB; for example, isoproterenol (˜30 nM) or calcium channel activators (−)BayK 8644 (300 nM); 4) Increase pacing to ˜80% of an appropriate physiological rate following the appearance of STA; 5) Treat with known or novel anti-arrhythmic agents to restore the fibrillating muscle to a condition in which it contracts in strict unison with the external stimulus.

### Example 6

### Novel Anti-arrhythmic Agents

As stated above, the disclosure provides novel anti-arrhythmic agents.

a. HA14-1

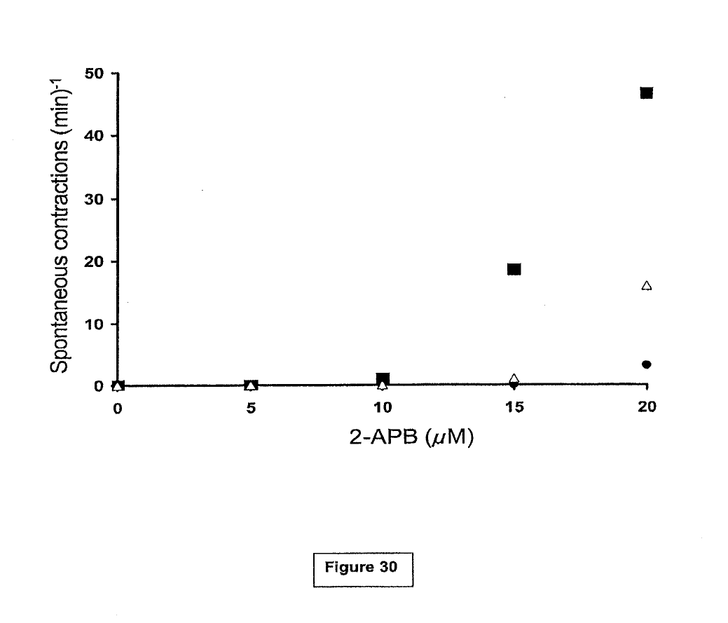

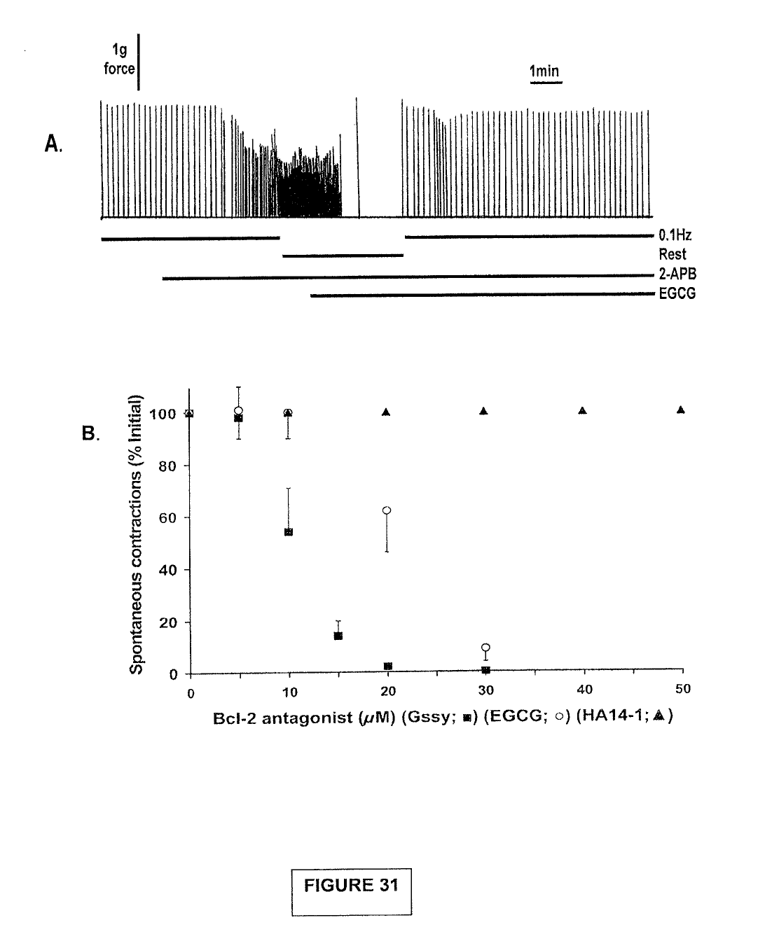

The compound HA14-1 (ethyl 2-amino-6-bromo-4-(1-cyano-2-ethoxy-2-oxoethyl)-4H-chromene-3-carboxylate) has been unexpectedly discovered to inhibit SMA and arrhythmia. To determine whether this compound inhibits SMA and arrhythmia, isolated rat left atrial appendages in KH perfusate were pre-treated with 30 μM HA14-1 or were left untreated. The appendages then were titrated with increasing concentrations of 2-APB, and SMA was measured and recorded. The muscles treated with HA14-1 displayed significantly fewer SMEs at concentrations of 15 μM and above than did the untreated appendages (FIG. 30, ). However, when these muscles were treated with HA14-1 10 min after exposure to various concentrations of 2-APB, there was no effect on SMA (FIG. 31, ▴). These data indicate that HA14-1 is effective to prevent SMA and arrhythmia, but that HA14-1 does not reverse SMA and arrhythmia under these conditions.

b. Gossypol

Gossypol (7-(8-formyl-1,6,7-trihydroxy-3-methyl-5-propan-2-ylnaphthalen-2-yl)-2,3,8-trihydroxy-6-methyl-4-propan-2-ylnaphthalene-1-carbaldehyde, CAS number 303-45-7) is a dimeric sesquiterpene found in cottonseed that has been unexpectedly discovered to prevent and reverse SMA and arrhythmia. Phytochemicals, especially polyphenols, have numerous applications as primary or adjuvant therapies. Most of their therapeutic potential is thought to occur because of their antioxidant properties or because of polyphenol activation of cell signaling pathways. Certain specific polyphenols, such as gossypol, bind to and modify the functional activity of bcl-2 (Kitada S. et al “Discovery, characterization, and structure-activity relationships studies of pro-apoptotic polyphenols targeting B-cell lymphocyte/leukemia-2 proteins.” J Medicinal Chemistry 46: 4259-4264 (2003).

Gossypol binds a hydrophobic pocket found on the surface of bcl-2 family proteins. This binding pocket represents a regulatory site, where endogenous antagonists dock onto bcl-2 and related anti-apoptotic proteins, negating their cytoprotective activity. (Fesik SW. “Insights into programmed cell death through structural biology.” Cell 103:273-282 (2000)). The endogenous antagonists bind via a conserved 16 amino acid motif known as the bcl-2 homology-3 (BH3) domain.

To determine the anti-arrhythmic activity of gossypol, isolated rat left atrial appendage and right ventricular muscle strips were used as model systems, as described above. Heart muscle was exposed to 20 μM 2-APB for 10 min to induce SMA, and rates of SMA were recorded as a percentage of initial SMA. The muscles then were titrated with up to 5 increasing concentrations of gossypol. Rates of SMA were recorded ˜10 min after the addition of concentration of the polyphenol.

As is shown in FIG. 31B, at concentrations of 10 μM and above, gossypol was observed to reduce SMA. At concentrations of 20 μM and above, SMA dropped to zero. Gossypol unexpectedly reverses SMA after it has been initiated, and does so within about 10 min of exposure of the muscles to gossypol. Thus gossypol may be useful as a treatment to reverse arrhythmia in subjects after the onset of an arrhythmia.

c. EGCG

It has been unexpectedly discovered that epigallocatechin gallate (“EGCG,” (−)-cis-2-(3,4,5-Trihydroxyphenyl)-3,4-dihydro-1(2H)-benzopyran-3,5,7-triol 3-gallate; CAS Number 989-51-5) is able to prevent and reverse SMA and arrhythmia. EGCG is the major component of the polyphenolic fraction of green tea. In its pure form, it is an odorless white, faint pink, or cream-colored powder or crystals. EGCG is available in research quantities from Sigma-Aldrich as two different preparations containing no less than 80% or no less than 95% of the compound, respectively, as determined by HPLC (Sigma-Aldrich, 1999). It is also available from Alexis Corporation at a purity of no less than 98% (Alexis Corporation, 2000; Fisher Scientific, 2000).

Phytochemicals, especially polyphenols, have numerous applications as primary or adjuvant therapies. Most of their therapeutic potential is thought to occur because of their antioxidant properties or because of polyphenol activation of cell signaling pathways. Certain specific polyphenols, such as EGCG, also bind to and modify the functional activity of bcl-2.

Those skilled in the art are able to safely administer EGCG to human subjects, and numerous studies have revealed the pharmacology of EGCG. An excellent source is the NIH sponsored review “SUMMARY OF DATA FOR CHEMICAL SELECTION (−) Epigallocatechin gallate,” issued by the National Toxins Program of the National Institute of Environmental Health Sciences (at http://ntp.niehs.nih.gov/ntp/htdocs/ChemBack ground/ExSumPdf/Epigallocatechingallate.pdf; last visited Jul. 8, 2008).

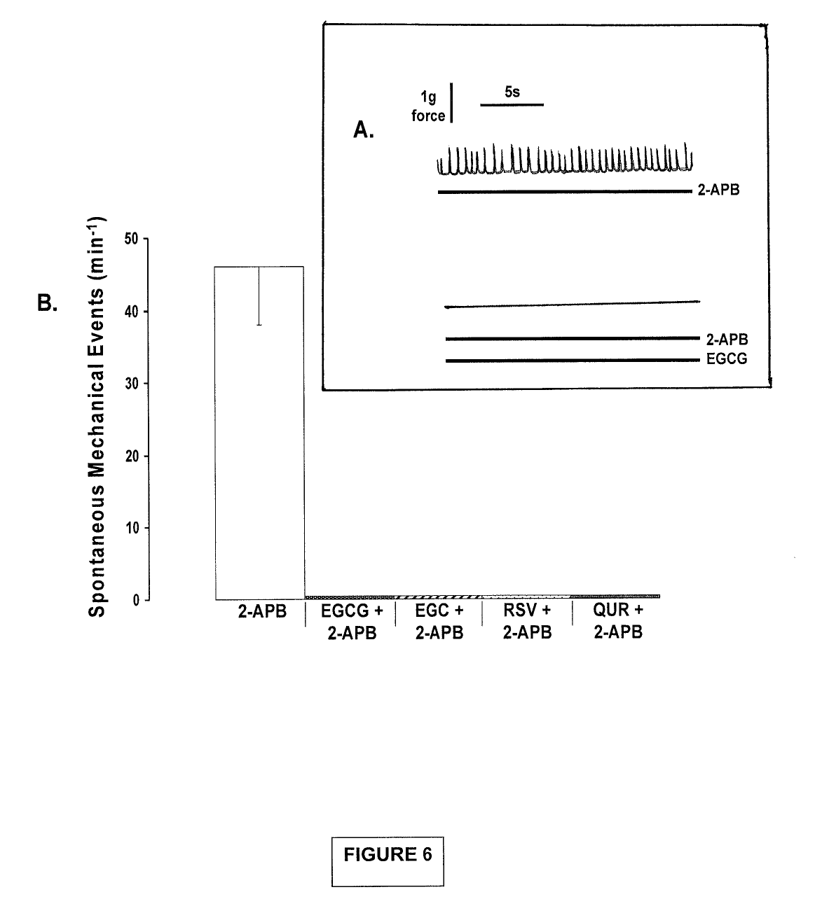

To determine whether EGCG can prevent arrhythmia, rat left atrial appendages (n=9) were exposed to 20 μM 2-APB and rates of SMA were recorded. Groups of muscles (n=6 per) were pre-treated with 30 μM EGCG for 10 min following which they were exposed to 2-APB. Rates of SMA were recorded 10 min later. After 10 min of exposure to 2-APB, rat left atrial appendage pre-treated with EGCG showed no SMEs (Data not shown). In negative controls, superfused rat left atrial appendages that were not pre-treated displayed about 45 SMEs per min (FIG. 6). EGCG is effective to prevent arrhythmia.

To determine whether EGCG is effective to reverse arrhythmia, isolated rat left atrial appendage and right ventricular muscle strips were used as model systems, as described above. Rat left atrial appendages were exposed to 20 μM 2-APB for 10 min to induce SMA, and rates of SMA were recorded as a percentage of initial SMA. The left atrial appendages then were titrated with up to 5 increasing concentrations of the following polyphenols: EGCG, gossypol, resveratrol, and quercetin. Rates of SMA were recorded ˜10 min after the addition of concentration of the polyphenol.

As is shown in FIGS. 31A and B, at concentrations of 20 μM and above, EGCG reduces SMA. EGCG unexpectedly reverses SMA after it has been initiated, and does so within about 10 min of exposure of the muscles to EGCG. Thus EGCG may be useful as a treatment to reverse arrhythmia in subjects after the onset of an arrhythmia.

d. EGC

Epigallocatechin (“EGC,” (−)-cis-2-(3,4,5-trihydroxyphenyl)-3,4-dihydro-1(2H)-benzopyran-3,5,7-triol; CAS number 970-74-1) is a polyphenol compound found in tea and chocolate that has unexpectedly been discovered to prevent SMA and arrhythmia. It has been studied as a possible anti-carcinogen and as a dietary antioxidant. EGC inhibits bcl-2 activity. Without wishing to be bound by any given hypothetical model, this may occur through post-translational modifications, such as phosphorylation, as polyphenols activate mitogen-activated protein kinase (MAPK) signaling and bcl-2 is among the downstream MAPK targets.

To determine whether EGC can prevent arrhythmia, left atrial appendages (n=9) were exposed to 20 μM 2-APB and rates of SMA were recorded. Groups of appendages (n=6 per) were pre-treated with 30 μM EGC for 10 min following which they were exposed to 2-APB. Rates of SMA were recorded 10 min later. After 10 min of exposure to 2-APB, the muscles pre-treated with EGC showed no SMEs (FIG. 6B). In negative controls, appendages that were not pre-treated displayed about 45 SMEs per minute. EGC is effective to prevent arrhythmia.

e. Quercetin

The compound known as quercetin (2-(3,4-dihydroxyphenyl)-3,5,7-trihydroxy-4H-chromen-4-one; CAS no. 117-39-5) is a member of a group of naturally occurring compounds, the flavonoids, which have a common flavone nucleus composed of two benzene rings linked through a heterocyclicpyrone ring. It has unexpectedly been discovered to prevent SMA and arrhythmia. Quercetin is found in various plants, food products, and dyes of natural origin. The estimated average daily intake of quercetin by an individual in the United States is 25 mg. Quercetin is the most abundant flavonol in the diet, and possesses biological activities such as antioxidative, and enzyme-inhibiting activities. Quercetin occurs in several different glycosidic forms in plants, but the flavanone glycosides are less numerous. It has been shown that quercetin is bioavailable from foods such as onions, tea and apples, which are its main dietary sources.

Quercetin has been tested on human and animal subjects in a clinical setting, and such published studies can be used by those skilled in the art to determine a dosing regimen. (e.g., Edwards R. et al. “Quercetin reduces blood pressure in hypertensive subjects” J. Nutrition 137: 2405-2411 (2007); “Toxicology and Carcinogenesis Studies of Quercetin (CAS No. 117-39-5) in F344 Rats (Feed Studies)” National Toxicology Program Technical Report Series, 409: 1-171 (1992).

To determine whether quercetin can prevent arrhythmia, rat left atrial appendages (n=9) were exposed to 20 μM 2-APB and rates of SMA were recorded. Groups of muscles (n=6 per) were pre-treated with 30 μM quercetin for 10 min following which they were exposed to 2-APB. Rates of SMA were recorded 10 min later. After 10 min of exposure to 2-APB, muscles pre-treated with quercetin showed no SMEs. In negative controls, superfused rat left atrial appendages that were not pre-treated displayed about 45 SMEs per min (FIG. 6B). Quercetin is effective to prevent arrhythmia.

Quercetin decreases the expression of bcl-2 in human cells (Marati R, et al. “Effects of quercetin on insulin-like growth factors (IGFs) and their binding protein-3 (IGFBP-3) secretion and induction of apoptosis in human prostate cancer cells” Journal of Carcinogenesis 5: 10 (2006); Nair H K et al, “Inhibition of Prostate Cancer Cell Colony Formation by the Flavonoid Quercetin Correlates with Modulation of Specific Regulatory Genes” Clinical and Diagnostic Laboratory Immunology 11: 63-69 (2003), which are hereby incorporated by reference for such teaching). Without wishing to be limited by any hypothetical model, this may be one mechanism by which quercetin prevents arrhythmia. An additional mechanism may be the induction by quercetin of post-translational modifications of bcl-2 or of a molecule or polypeptide regulated by bcl-2; MAPK phosphorylation of bcl-2 being one exemplar of this mechanism.

f. 2-Methoxy Antimycin A

To determine whether 2-methoxy antimycin A (2MAA) prevents SMA and arrhythmia, appendages were pre-treated with 2MAA (80 μM) or were left untreated. Appendages were titrated with increasing concentrations of 2-APB and rates of SMA were recorded. At 2-APB concentrations of 10 μM and below no SMA was observed. At 15 μM 2-APB, the untreated appendages showed about 20 SMEs per min, whereas the 2MAA-treated appendages showed none. At 20 μM 2-APB, the untreated appendages showed about 50 SMEs per min, whereas the 2MAA-treated appendages showed between 15 and 20 SMEs per min (FIG. 30, Δ). Depending on the conditions, 2MAA has been unexpectedly discovered to either completely prevent SMA and arrhythmia, or significantly inhibit it.

g. Resveratrol

Resveratrol (5-[2-(4-hydroxyphenyl)ethenyl]benzene-1,3-diol; CAS no. 501-36-0) is a polyphenol that occurs naturally in grapes, peanuts, and a number of other plants that has unexpectedly been discovered to prevent arrhythmia. It is found in foods/drinks made from grapes and peanuts, and also in a number of herbal remedies, both alone and as part of plant extracts. Resveratrol is produced commercially by several companies. A commercial extraction method involves using alcohol and water to produce resveratrol from Polygonum cuspidatum. Resveratrol compounds may be produced or extracted for research purposes by treating cell suspension cultures of grapes with a natural substance from a fungus. (Haneke K E “trans-Resveratrol [50]-36-0] Review of Toxicological Literature” National Institute of Environmental Health Sciences, Research Triangle (2002)).

Resveratrol has been tested on human and animal subjects in a clinical setting, and such published studies can be used by those skilled in the art to determine a dosing regimen. A review of the toxicological literature sponsored by the National Institute of Environmental Health Sciences is one excellent source (Haneke K E “trans-Resveratrol [50]-36-0] Review of Toxicological Literature” National Institute of Environmental Health Sciences, Research Triangle, (2002)).

To determine whether resveratrol can prevent arrhythmia, rat left atrial appendages (n=9) were exposed to 20 μM 2-APB and rates of SMA were recorded. Groups of muscles (n=6 per) were pre-treated with 30 μM resveratrol for 10 min following which they were exposed to 2-APB. Rates of SMA were recorded 10 min later. After 10 min of exposure to 2-APB, muscles pre-treated with resveratrol showed no SMEs. In negative controls, appendages that were not pre-treated displayed about 45 SMEs per min (FIG. 6B). It was unexpectedly discovered that resveratrol is effective to prevent arrhythmia.

h. ABT-737

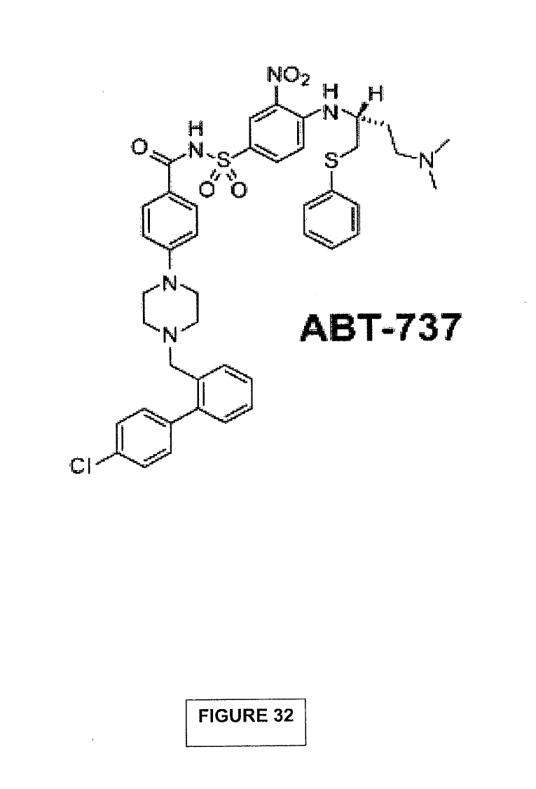

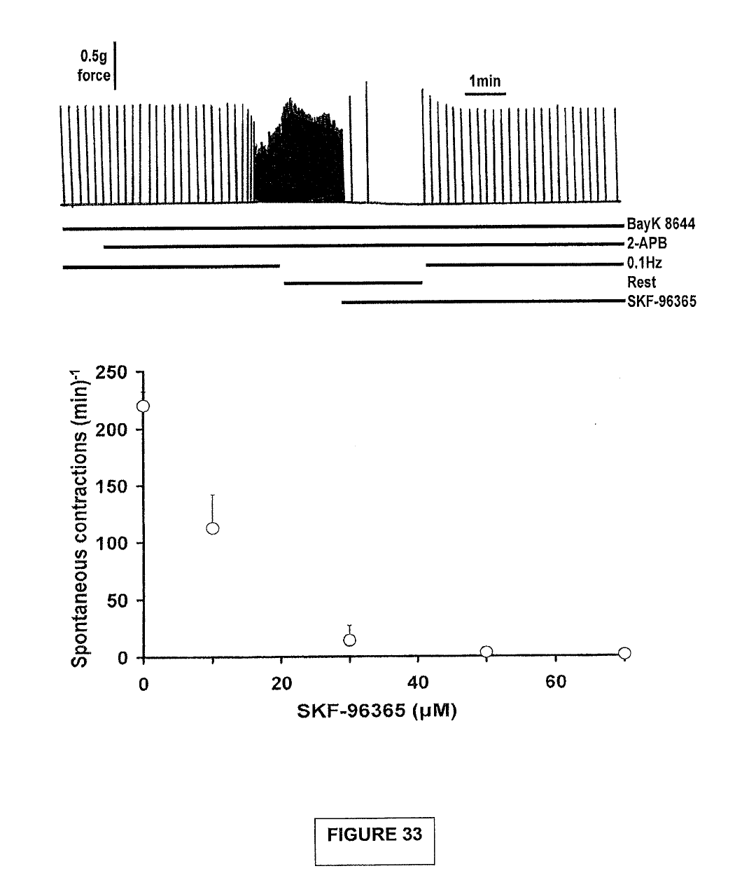

The compound ABT-737 is another potent inhibitor of bcl-2 (see FIG. 32 for the structure of this molecule). ABT-737 is a potent anti-tumor agent, and its properties as an inhibitor of bcl-2 make it well suited for use as an anti-arrhythmic agent. It can be prepared as described by Oltersdorf et al. “An inhibitor of Bcl-2 family proteins induces regression of solid tumours” Nature 435: 677-681 (2002).

i. SKF-96365

The compound SKF-9635 (1-[2-(4-Methoxyphenyl)-2-[3-(4-methoxyphenyl) propoxy]ethyl-1H-imidazole hydrochloride) is an inhibitor of the SOC, a downstream bcl-2 target. We unexpectedly discovered it prevents and reverses SMA and STA.

FIG. 33 (upper panel, left side) shows the mechanical function of a 0.1 Hz-paced left atrial appendage treated with BayK 8644 and 2-APB to induce STA. ˜50 μM SKF-96365 was added to the muscle bath (SKF-96365; upper panel, middle) and STA was suppressed, restoring normal pacing-induced mechanical contractions that occurred in a one-to-one manner (upper panel, right side; 0.1 Hz). FIG. 33, lower panel shows a summary of SKF-96365 reversal of STA with an IC50 of ˜15 μM, a concentration similar to that reported for its inhibition of the SOC. Other experiments (data not shown) reveal SKF-96365 also (ii) prevents STA and (iii) prevents and reverse SMA.

### Example 7

### 2-APB Induces STA in Left Atrial Appendages Exposed to ATXII

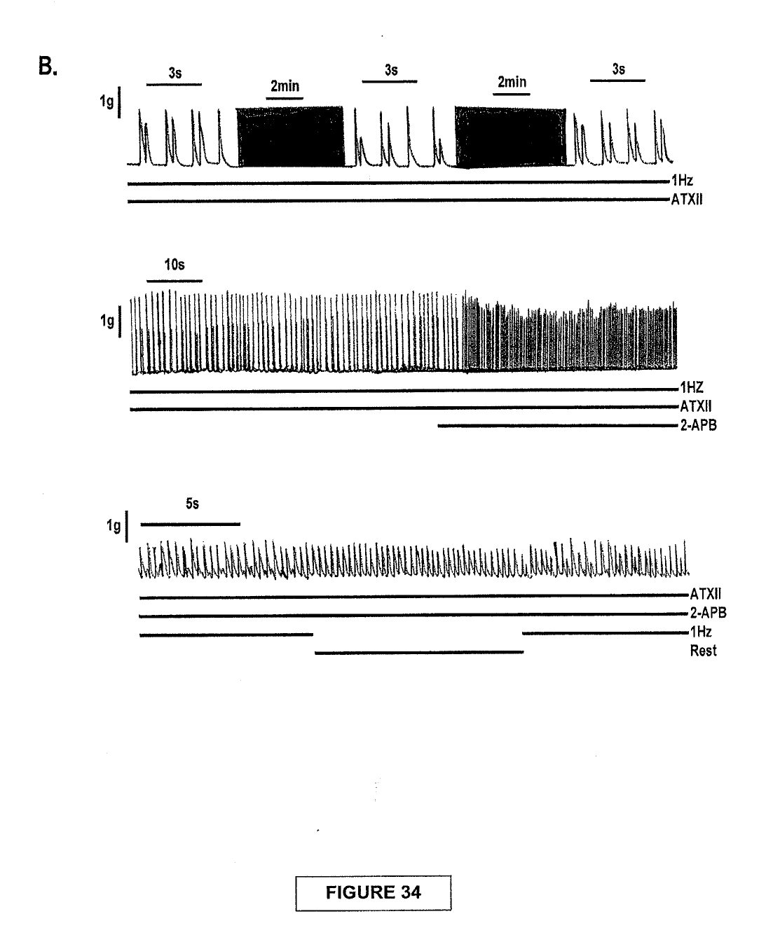

Prior art suggests that the transition from triggered afterdepolarizations to abnormal automaticity requires accelerated SR ryanodine receptor calcium leakage. It was tested whether the altered voltage-independent calcium homeostasis which 2-APB provokes is sufficient to initiate frank automaticity from triggered activity. To this end ATXII which increases late sodium current, prolongs action potential duration (FIG. 3B, middle panel), and increases myocyte calcium concentration was used. Left atrial appendages exposed to <50 nM ATXII produced a steady-state of triggered aftercontractions (FIG. 34; Upper panel). In support of the disclosed model, STA began to occur in these muscle soon after the addition of ˜25 μM 2-APB (FIG. 34; lower panel). The severity of this automatic activity (FIG. 34; lower panel: Rest) is markedly greater and more disorganized compared to muscle undergoing STA with normal action potential duration. Thus 2-APB is sufficient to produce the transition from triggered afterdepolarizations to automatic activity under conditions that prolong action potential duration and increase calcium loading.

### Example 8

### Depletion of the Left Atrial Caffeine-Sensitive Calcium Pool but not the Ryanodine-Sensitive Pool Suppresses STA

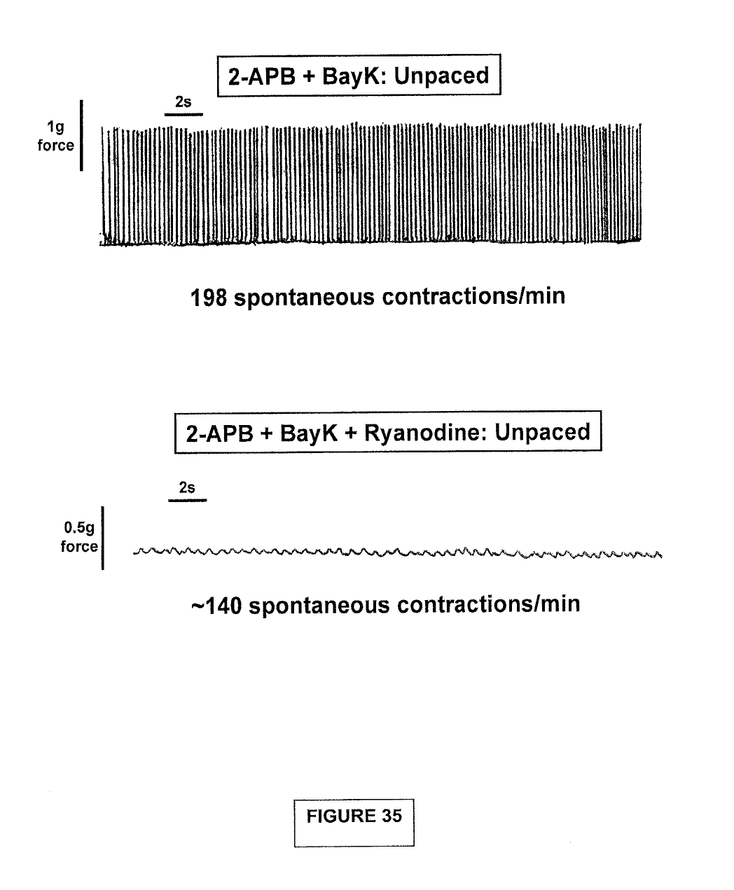

Prior art suggests that leakage of calcium stores regulated by the ryanodine receptor calcium release channel provokes abnormal triggered and automatic activity. To test whether STA requires the SR ryanodine receptor regulated calcium pool, superfused rat left atrial appendages were exposed to BayK 8644 and then to 2-APB to provoke STA (FIG. 35; upper panel). The rate of STA was recorded. These muscles then were exposed to 600 nM ryanodine. This alkaloid specifically binds to the ryanodine receptor and locks it in an open conformation, causing the leakage and the depletion of the SR calcium stores required for excitation-contraction coupling. This decreases left atrial appendage mechanical function (compare the scales of force of contraction in FIG. 35; upper and lower panels). STA, however, persists even in the presence of ryanodine and depleted SR calcium stores (FIG. 35; lower panel).

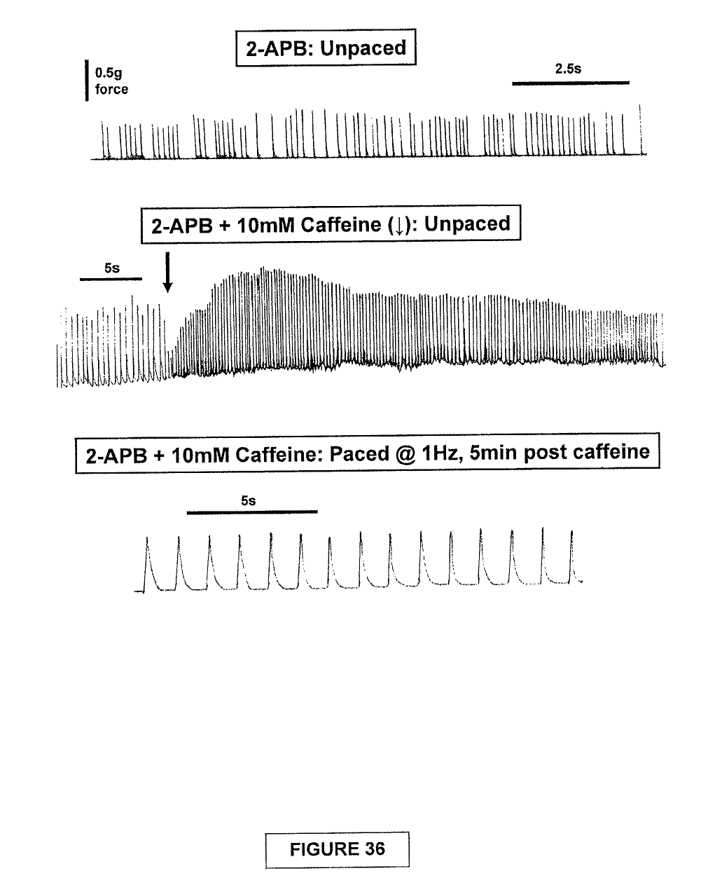

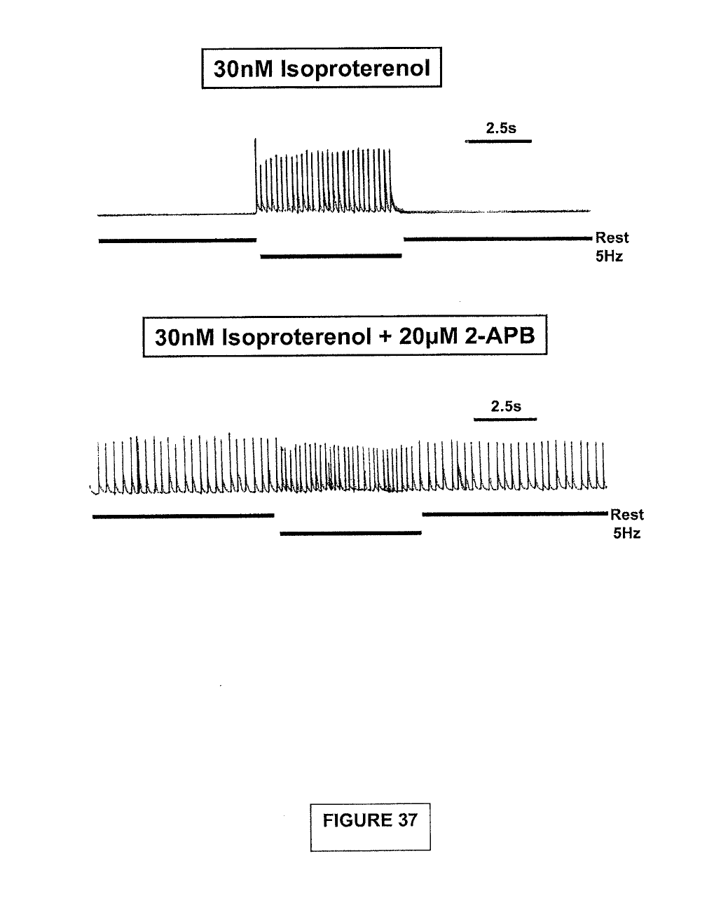

It was next tested whether SMA or STA require ryanodine-insensitive cardiac calcium stores. To this end, SMA was induced in left atrial appendages and the pacing stimulus was stopped. These left atria then were washed with KH containing ˜25 μM 2-APB and 10 mM caffeine. Caffeine causes the emptying of intracellular calcium stores linked to the IP3R2 and causes leakiness in the SR ryanodine receptor. Caffeine treatment produced two effects. First, immediately after exposure to caffeine and 2-APB, the rate of left atrial SMA markedly increased (FIG. 36; compare upper and middle panels) and then ceased completely shortly thereafter (FIG. 36; right of the middle panel). These quiescent (caffeine and 2-APB)-treated muscles still responded to pacing stimuli (FIG. 36; lower panel) indicating that normal excitation-contraction coupling remains intact here.

FIGS. 35 and 36 show that myocyte internal calcium stores drive both SMA and the transition from SMA to STA; in contrast to prior art, this store is not the ryanodine receptor pool of calcium, rather it is a ryanodine-insensitive pool of calcium which caffeine depletes. Furthermore, these figures indicate that SMA and STA require ryanodine-insensitive calcium stores and not the ryanodine-sensitive calcium stores that control the strength of muscle force development following external stimulation.

### Example 9

### STA does not Require External Stimulation but is Sensitive to Manipulations that Affect Left Atrial Transmembrane Voltage and the Sodium Channel

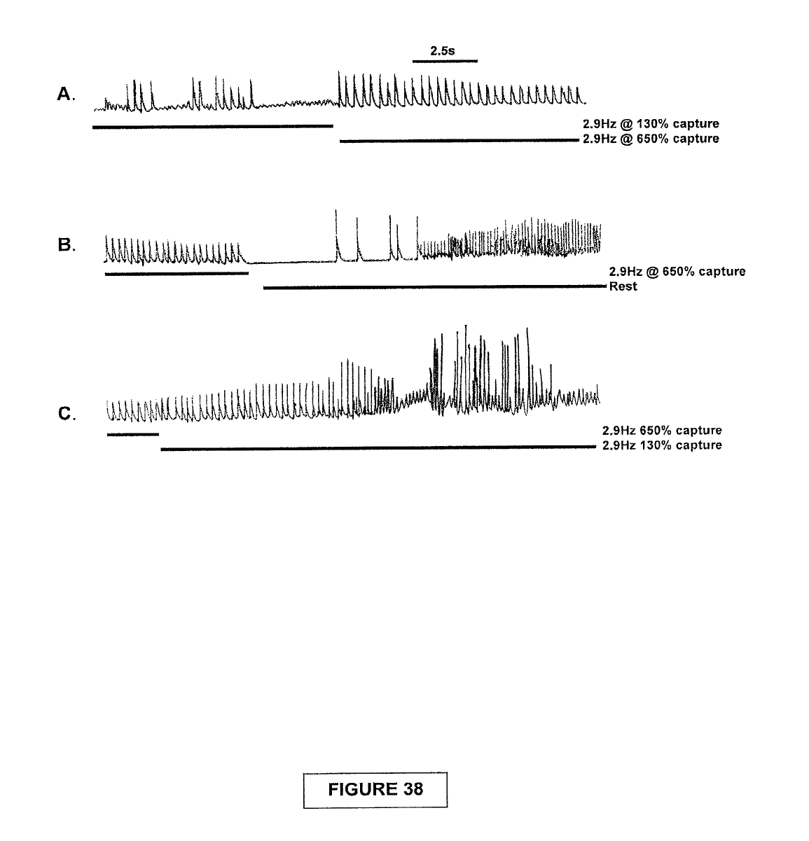

2-APB activates automatic electromechanical activity which does not require an external depolarizing stimulus. It was queried how the 2-APB-induced, voltage-independent STA system interacts with the normal voltage-dependent system that external pacing activiates. Left atrial appendages were treated with 30 nM isoproterenol alone, and then the pacing stimulus was stopped (FIG. 37; upper panel). As expected, this normal, non-automatic muscle became quiescent in the absence of pacing. Applying a rapid ˜5 Hz pacing stimulus produced the expected one-to-one capture and mechanical response.

Superfused left atrial appendages exposed to 30 nM isoproterenol and ˜25 μM 2-APB exhibit STA (FIG. 37; left side lower panel). Imposing a ˜5 Hz pacing on these abnormally automatic atria also produces one-to-one capture and mechanical response (FIG. 37; center of the lower panel: 5 Hz). Turning off the pacing stimulus shortly thereafter restores automatic STA (FIG. 37; right side of the lower panel).

These data demonstrate that automatic STA can be overdriven and temporarily suppressed by a rapid external pacing stimulus. This result is a fundamental property of in vivo tachyarrhythmias in that prior art has shown in vivo tachycardic or fibrillatory foci can be overdriven and electrically captured by a rapid pacing stimulus. Overdrive capture is temporary during STA because 2-APB, the chemical impetus for automaticity, remains in the solution to activate the voltage-independent system responsible for STA.

Two additional experiments further demonstrate the interaction between automatic STA and the normal non-automatic excitation system.

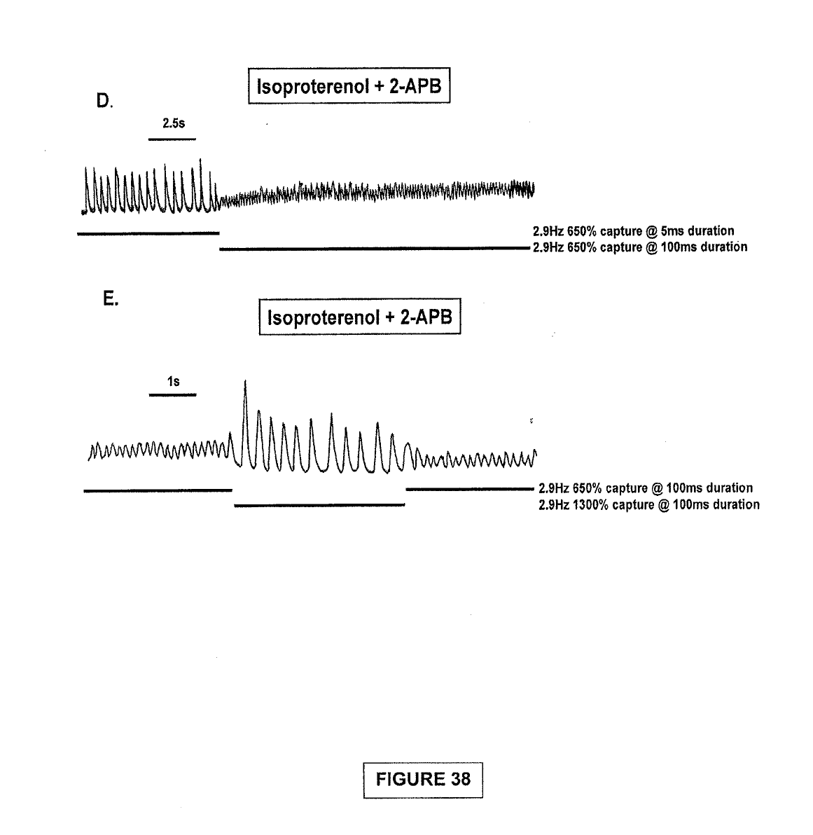

First, left atrial appendages undergoing STA were exposed to a pacing stimulus set to ˜80% of the rate of STA, 130% of the capture voltage and a short 5 millisecond (ms) stimulus duration. Capture voltage is the voltage required to stimulate a non-automatic muscle to contract. When (i) STA and (ii) pacing stimulated impulses impinge on the left atrial appendage at the same time they produced disorganized, chaotic mechanical activity reminiscent of fibrillation (FIG. 38; left side of panel A). If the strength of the pacing stimulus is increased from 130% of the capture voltage to 650% of the capture voltage, the chaotic activity of these muscles becomes organized, and one-to-one capture with the pacing stimulus occurs. Thus, (i) high frequency pacing and (ii) increased strength of pacing stimulus can suppress STA.

If the high voltage (650%) capture pacing stimulus is stopped, then regular STA soon reappears (FIG. 38; panel B). This suggests that high capture voltage stimulation temporarily suppresses STA. Importantly, if the high voltage (650%) pacing stimulus is decreased back to 130% of capture voltage, ‘fibrillation’ reappears presumably because STA and the external pacing stimuli are again brought into conflict (FIG. 38; panel B). These results suggest that STA drives disorganized chaotic mechanical activity.

Second, subjecting left atrial appendages undergoing STA to a single pulse of long duration (>50 ms) electrical stimulation at ˜650% of capture voltage produced rapid fibrillation in these preparations (FIG. 38; panel C). This frank fibrillation can be stopped and STA re-initiated if the fibrillating muscle is subjected to a single stimulus whose voltage is ˜1000% of the capture voltage (FIG. 38; panel D).

Thus external stimulation which opens the transient sodium channel can (i) suppress STA, (ii) trigger fibrillation from STA, and (iii) defibrillate intact heart muscle; together these results show that the voltage-independent system we claim here is an important regulator of arrhythmia in heart muscle.

FIGS. 37 and 38 show that while STA does not require external stimulation to occur, it is sensitive to manipulations that affect cardiac voltage-dependent sodium channel activity. Thus the STA system we claim here interacts with the voltage-dependent system to provoke chaotic, fibrillation-like activity.

These examples allow a further development of the proposed model (FIG. 7B). Without being limited to other mechanisms and without limiting the scope of the present disclosure, these examples show that SOC or voltage-independent calcium entry coupled with bcl-2 regulated calcium leakage from a ryanodine-insensitive calcium store leads to changes in the functional properties of a cardiac sodium channel. The normal sodium channel requires an external depolarizing impulse to transiently open and depolarize the myocyte cell (FIG. 7B; I_{NaT} dark ellipsoid). During STA, the sodium channel passes from this normal voltage-gated form to a form, the so-called ‘persistently activate sodium channel’, whose activity is driven by cell calcium, (FIG. 7B; I_{NaP}, light ellipsoid). Automatic STA is the result (FIG. 7B; metronome on the right side). FIGS. 36, 37 & 38 show these two forms are in a dynamic equilibrium. Either rapid external pacing or an extraordinarily strong external stimulus disturbs this equilibrium to produce a temporary (with 2-APB or a related activator continually present) or a more permanent reversion of abnormal automatic activity during in vivo arrhythmia (FIG. 7B; inset: Rate, ↑V). Pharmacological analyses using ranolazine, lidocaine, and flecainide show that I_{Nap} is the end arrhythmic target for bcl-2 linked changes in IP3R/SOC calcium homeostasis.

1. A method of treating or preventing arrhythmia in a subject in need thereof, said method comprising administering to the subject a therapeutically effective amount of a compound that inhibits that inhibits the activity or expression of bcl-2 or a bcl-2 target.

2-68. (canceled)

69. A method of treating or preventing arrhythmia in a subject in need thereof, said method comprising administering to the subject a therapeutically effective amount of a compound that inhibits an ion channel active during an arrhythmic event, wherein the ion channel is selected from the group consisting of a sodium channel and a voltage-independent calcium channel.

70. The method of claim 69, wherein the voltage-independent calcium channel is a calcium channel regulated by intracellular calcium stores or an inositol 1,4,5-triphosphate receptor.

71. The method of claim 69, wherein the sodium channel is activated by an increase in intracellular calcium.

72. The method of claim 69, wherein the compound is a small molecule, a functional nucleic acid, an immunoglobulin, a fragment of an immunoglobulin molecule, a chimeric immunoglobulin, a fragment of a chimeric immunoglobulin, a polymer of immunoglobulin molecules, a peptidomimetic or a combination of the foregoing.

73. A pharmaceutical composition for the treatment or prevention of arrhythmia in a subject, said composition comprising a compound that inhibits the activity or expression of bcl-2 or a bcl-2 target or a compound that inhibits cellular voltage-independent calcium homeostasis.

74. An assay for the identification of an anti-arrhythmic agent comprising:
(a) exposing a myocyte to an arrhythmic agent at an arrhythmia-inducing effective concentration;
(b) exposing the myocyte to a candidate anti-arrhythmic agent;
(c) determining a parameter indicative of arrhythmia in the myocyte in the presence of the arrhythmic agent and in the presence of the arrhythmic agent and the candidate anti-arrhythmic agent; and
(d) comparing the parameter indicative of arrhythmia in the myocyte determined in the presence of the arrhythmic agent and in the presence of the arrhythmic agent and the candidate anti-arrhythmic agent, wherein an improvement in the parameter indicative of arrhythmia determined in the presence of the arrhythmic agent and the anti-arrhythmic agent indicates the identification of an anti-arrhythmic agent.

75. The assay of claim 74, wherein the myocyte is an isolated myocyte, a component of a cardiac muscle or a component of an intact heart.

76. The assay of claim 75, wherein the cardiac muscle is selected from the group consisting of: a perfused heart, a portion of a perfused heart, a portion of a heart, a left atrial appendage, a ventricular muscle strip, and a right ventricular muscle strip.

77. The assay of claim 74, further comprising increasing the intracellular calcium concentration of the myocyte.

78. The assay of claim 74, wherein the arrhythmic agent is 2-APB, an analog of 2-APB, or a physiologically acceptable salt of 2-APB.

79. The assay of claim 74, wherein the exposure of the myocyte to the candidate anti-arrhythmic agent occurs before exposure of the myocyte to the arrhythmic agent, after exposure of the myocyte to the arrhythmic agent, or simultaneously with exposure of the myocyte to the arrhythmic agent.

80. An assay for the identification of an anti-arrhythmic agent, said assay comprising:
(a) contacting a polypeptide with a candidate anti-arrhythmic agent, wherein the polypeptide is selected from the group consisting of: bcl-2 and a bcl-2 target; and
(b) measuring the binding between the polypeptide and the candidate anti-arrhythmic agent, wherein binding modulates bcl-2 or bcl-2 target function and indicates the identification of an anti-arrhythmic agent.

81. The assay of claim 80, wherein the bcl-2 target is an inositol 1,4,5-triphosphate receptor, a store operated calcium channel, a persistent sodium channel, or a combination of the foregoing.

82. A method of diagnosing risk for arrhythmia or SMA, said method comprising:
(a) obtaining a biological sample from a first subject whose risk for arrhythmia or SMA is to be determined;
(b) measuring a level of expression or activity of bcl-2 or a bcl-2 target in the first subject; and
(c) comparing the level of expression or activity in the first subject to a level of expression or activity measured in a control subject, wherein an increase in the level of expression or activity indicates an increased risk for arrhythmia or SMA.

83. The assay of claim 82, wherein the myocyte is an isolated myocyte, a component of a cardiac muscle, or a component of an intact heart.

84. The assay of claim 83, wherein the cardiac muscle is selected from the group consisting of: a perfused heart, a portion of a perfused heart, a left atrial appendage, a ventricular muscle strip and a right ventricular muscle strip.

85. The assay of claim 82, further comprising increasing the intracellular calcium concentration of the myocyte.
